(19) United States
(12) Patent Application Publication
(10) Pub. No.: US 2016/0122312 A1
(43) Pub. Date: May 5, 2016

(54) ANTI-VIRAL COMPOUNDS, PHARMACEUTICAL COMPOSITIONS AND METHODS OF USE THEREOF

(71) Applicant: KINETA, INC., Seattle, WA (US)

(72) Inventors: Shawn P. Iadonato, Seattle, WA (US); Kristin M. Bedard, Bellevue, WA (US); Kerry W. Fowler, Seattle, WA (US)

(21) Appl. No.: 14/895,899

(22) PCT Filed: Jul. 16, 2014
(86) PCT No.: PCT/US14/46829

§ 371 Date: Dec. 3, 2015

Related U.S. Application Data
(60) Provisional application No. 61/846,997, filed on Jul. 16, 2013.
(60) Provisional application No. 61/991,417, filed on May 9, 2014.

(51) Int. Cl. C07D 311/36; A61K 39/21; A61K 39/39; A61K 39/155; A61K 39/13; A61K 39/29; C07D 311/22; A61K 39/145

(52) U.S. Cl. CPC C07D 311/36; C07D 311/22; A61K 39/21; A61K 39/145; A61K 39/155; A61K 39/13; A61K 39/29; A61K 39/39; A61K 2039/55511

(57) ABSTRACT

Disclosed herein are compounds, pharmaceutical compositions, and methods for the treatment of viral infection, including RNA viral infection, as well as compounds, pharmaceutical compositions, and methods for modulating the RIG-I pathway in a subject and/or in cells. These compounds are isoflavone derivatives, typically substituted at the 3-position with an aryl group and at the 7-position with a heterofunctional group.

### CROSS REFERENCES TO RELATED APPLICATIONS

This application claims priority to U.S. Provisional Patent Application Ser. No. 61/846,997 filed Jul. 16, 2013, and U.S. Provisional Patent Application Ser. No. 61/991,417 filed May 9, 2014, the entire contents of both of which are incorporated by reference herein.

### STATEMENT OF GOVERNMENT INTEREST

This invention was made with government support under National Institutes of Health Grant No. A1081335. The government has certain rights in the invention.

### FIELD OF THE DISCLOSURE

The disclosure provides compounds, pharmaceutical compositions, and methods for treating viral infection, among other uses. The compounds modulate the retinoic acid-inducible gene 1 (RIG-I) pathway.

### BACKGROUND OF THE DISCLOSURE

Viruses, such as RNA viruses, represent an enormous public health problem in the United States and worldwide. Well-known RNA viruses include influenza virus (including the avian and swine isolates; also known as flu), hepatitis C virus (HCV), West Nile virus (WNV), SARS-coronavirus (SARS), respiratory syncytial virus (RSV), and human immunodeficiency virus (HIV).

As one example, more than 170 million people worldwide are infected by HCV, and 130 million of these are chronic carriers at risk of developing chronic liver diseases (cirrhosis, carcinoma, and liver failure). As such, HCV is responsible for two thirds of all liver transplants in the developed world. Recent studies show that the death rate from HCV infection is rising due to the increasing age of chronically infected patients. As a second example, seasonal flu infects 5-20% of the population annually resulting in 200,000 hospitalizations and 36,000 deaths each year.

Compared to HCV and influenza, WNV causes the lowest number of infections, 981 in the United States in 2010. Twenty percent of infected patients, however, develop a severe form of the disease, resulting in a 4.5% mortality rate. Unlike HCV and influenza, there are no approved therapies for the treatment of WNV infection, and it is a high-priority pathogen for drug development due to its potential as a bioterrorist agent.

Among the viruses listed, vaccines exist only for influenza virus. Accordingly, drug therapy is essential to mitigate the significant morbidity and mortality associated with these viruses. Unfortunately, the number of antiviral drugs is limited, many are poorly effective, and nearly all are plagued by the rapid evolution of viral resistance and a limited spectrum of action. Moreover, treatments for acute HCV and influenza infections are only moderately effective. The standard of care for HCV infection, PEGylated interferon and ribavirin, is effective in only 50% of patients, and there are a number of dose-limiting side effects associated with the combined therapy. Both classes of acute influenza antivirals, adamantanes and neuraminidase inhibitors, are only effective within the first 48 hours after infection, thereby limiting the window of opportunity for treatment. High resistance to adamantanes already restricts their use, and massive stockpiling of neuraminidase inhibitors will eventually lead to overuse and the emergence of resistant strains of influenza.

Most drug development efforts against viruses target viral proteins. This is a large part of the reason that current drugs are narrow in spectrum and subject to the emergence of viral resistance. As most RNA viruses have small genomes and many encode less than a dozen proteins, viral targets are limited. Based on the foregoing, there is an immense and unmet need for effective treatments against viral infections, including RNA viral infections.

### SUMMARY OF THE DISCLOSURE

The compounds, pharmaceutical compositions, and methods disclosed herein shift the focus of viral drug development away from the targeting of viral proteins to the targeting and enhancing of the host's innate antiviral immune response. Such compounds, pharmaceutical compositions, and methods are likely to be more effective, be less susceptible to the emergence of viral resistance, cause fewer side effects, and be effective against a range of different viruses. Tan, S. L., et al. (2007) Systems biology and the host response to viral infection, Nat Biotechnol 25, 1383-1389.

The retinoic acid-inducible gene 1 (RIG-I) pathway is intimately involved in regulating the innate immune response to virus infections including RNA virus infections. RIG-I agonists are expected to be useful for the treatment of many viruses including Hepatitis C Virus (HCV), influenza virus, and West Nile virus (WNV), among others. Accordingly, the present disclosure relates to compounds, pharmaceutical compositions including the compounds, and associated methods of use to treat viral infection, including RNA viral infection, wherein the compounds modulate the RIG-I pathway.

The compounds have the following general chemical structure

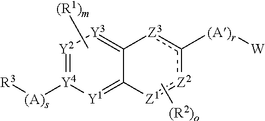

as described more fully in the Detailed Description.

### BRIEF DESCRIPTION OF THE DRAWINGS

FIGS. 1A, 1B, and 1C show the antiviral activity of the compounds KIN100 and KIN101 against HCV. (A) HCV focus-forming assay done in Huh7 cells pre-treated with KIN100 for 24 hours and infected with HCV2a at a multiplicity of infection (MOI) of 0.5 for 48 hours. HCV proteins were detected by immunofluorescent staining with viral-specific serum and foci were normalized to negative control cells that were not drug treated (equal to 1). (B) Quantitation of HCV viral RNA by real-time quantitative PCR (RT-qPCR) done in Huh7 cells pre-treated with KIN101 for 18 hours and infected with HCV2a at MOI of 1.0 for 72 hours. Viral RNA was isolated and quantitated in the supernatant of infected cultures. (C) A similar quantitation of HCV viral RNA by RT-qPCR done in Huh7 cells infected with HCV2a at MOI of 1.0 for 4 hours and then treated with KIN101.

FIGS. 2A and 2B show the antiviral activity of the compound KIN101 against RSV. (A) Cell viability following infection with RSV A2 and treatment with KIN101. (B) KIN101 treatment decreased RSV viral RNA 48 hours post infection in cells treated with KIN101.

FIGS. 3A, 3B, and 3C show results from the influenza focus-forming assay. Decrease in foci is graphed as percent inhibition of viral infection by compound. (A) KIN101 showed dose-dependent decrease in viral infection of 293 cells; derivative compounds KIN134, KIN263, KIN267, KIN269, KIN282, KIN291, KIN308, and KIN306 improved on this antiviral activity as shown by decreased viral titer. (B) KIN328, KIN371, KIN372, KIN376, KIN385, KIN392, KIN269, KIN394, KIN395, and KIN299 showed dose-dependent decrease in viral infection of 293 cells. (C) Determined 1050 values of exemplary derivative compounds in the influenza antiviral assay.

FIGS. 4A and 4B show the antiviral activity of selected compounds against Dengue virus (DNV). (A) Dose-dependent decrease in viral protein in cells infected with DNV and treated with increasing amounts of KIN101. (B) Results of the DNV focus-forming assay for antiviral activity. Decrease in foci is graphed as percent inhibition of viral infection by compound. The compounds KIN101 (black dashed line), KIN134, KIN269, KIN328, KIN372, KIN376, and KIN385 showed dose-dependent decrease in viral infection of Huh7 cells. 1050 values (in M) are shown.

FIGS. 5A and 5B show the antiviral activity of selected compounds against human cytomegalovirus (hCMV). (A) Dose-dependent decrease in hCMV as measured by foci (FFU/mL) in samples treated with KIN385, KIN392, KIN394, and KIN395. (B) Dose-dependent decrease in hCMV as measured by foci (FFU/mL) in samples treated with KIN269, KIN134, KIN372, KIN328, and KIN376.

FIG. 6 shows interferon regulatory factor-3 (IRF-3) responsive gene expression induced by the compound KIN269 in 293 cells. Influenza infection was used as a positive control for induction of gene expression.

FIGS. 7A-7E show in vivo broad spectrum antiviral activity and bioavailability of KIN269. KIN269 (10 mg/kg in 10% HPBCD) intranasal treatment reduces replication and titer of influenza (A) mouse hepatitis virus (MHV) (B) in the lung. (C) KIN269 serum levels over time when dosed at 10 mg/kg via intraperitoneal injection or intravenous injection. (D) KIN269 inhibited DNV as measured in serum when dosed IP 10 mg/kg/day. (E) KIN269 (20 mg/kg) inhibited flu replication in the lung when administered by intranasal instillation either −24 hours prior (prophylactic) or +24 hours post (therapeutic) lethal infection with PR8 flu. Lung tissue was harvested 72 hours after infection and flu RNA was quantitated by PCR.

### DETAILED DESCRIPTION

The present disclosure provides compounds, pharmaceutical compositions, and methods that shift the focus of viral treatments away from the targeting of viral proteins to the targeting and enhancing the host (subject's) innate antiviral immune response. Such compounds, pharmaceutical compositions, and methods are likely to be more effective, less susceptible to the emergence of viral resistance, cause fewer side effects, and be effective against a range of different viruses. Tan, S. L., et al. (2007) Systems biology and the host response to viral infection, Nat Biotechnol 25, 1383-1389.

The retinoic acid-inducible gene 1 (RIG-I) pathway is intimately involved in regulating the innate immune response to virus infections including RNA virus infections. RIG-I is a cytosolic pathogen recognition receptor that is essential for triggering immunity to a wide range of RNA viruses. Li, K., et al. (2005) Distinct poly(I-C) and virus-activated signaling pathways leading to interferon-beta production in hepatocytes, J Biol Chem 280, 16739-16747; Loo, Y. M., et al. (2008) Distinct RIG-I and MDA5 signaling by RNA viruses in innate immunity, J Virol 82, 335-345; Loo, Y. M., et al. (2006) Viral and therapeutic control of IFN-beta promoter stimulator 1 during hepatitis C virus infection, Proc Natl Acad Sci USA 103, 6001-6006; Saito, T., et al. (2007) Regulation of innate antiviral defenses through a shared repressor domain in RIG-I and LGP2, Proc Natl Acad Sci USA 104, 582-587. RIG-I is a double-stranded RNA helicase that binds to motifs within the RNA virus genome characterized by homopolymeric stretches of uridine or polymeric U/A motifs. Saito, T., et al. (2008) Innate immunity induced by composition-dependent RIG-I recognition of hepatitis C virus RNA, Nature 454, 523-527. Binding to RNA induces a conformation change that relieves RIG-I signaling repression by an autologous repressor domain, thus allowing RIG-I to signal downstream through its tandem caspase activation and recruitment domains (CARDs). Johnson, C. L., et al. (2006) CARD games between virus and host get a new player, Trends Immunol 27, 1-4.RIG-I signaling is dependent upon its NTPase activity, but does not require the helicase domain. Sumpter, R., Jr., et al. (2005) Regulating intracellular antiviral defense and permissiveness to hepatitis C virus RNA replication through a cellular RNA helicase, RIG-I, J Virol 79, 2689-2699; Yoneyama, M., et al. (2004) The RNA helicase RIG-I has an essential function in double-stranded RNA-induced innate antiviral responses, Nat Immunol 5, 730-737. RIG-I signaling is silent in resting cells, and the repressor domain serves as the on-off switch that governs signaling in response to virus infection. Saito, Proc Natl Acad Sci USA 104, 582-587.

Without being bound by a theory or particular mechanism of action, RIG-I signaling is transduced through IPS-1 (also known as Cardif, MAVs, and VISA), an essential adaptor protein that resides in the outer mitochondrial membrane. Kawai, T., et al. (2005) IPS-1, an adaptor triggering RIG-I- and Mda5-mediated type I interferon induction, Nat Immunol 6, 981-988; Meylan, E., et al. (2005) Cardif is an adaptor protein in the RIG-I antiviral pathway and is targeted by hepatitis C virus, Nature 437, 1167-1172; Seth, R. B., et al. (2005) Identification and characterization of MAVS, a mitochondrial antiviral signaling protein that activates NF-kappaB and IRF 3, Cell 122, 669-682; Xu, L. G., et al. (2005) VISA is an adapter protein required for virus-triggered IFN-beta signaling, Mol Cell 19, 727-740. IPS-1 recruits a macromolecular signaling complex that stimulates the downstream activation of interferon regulatory factor-3 (IRF-3), a transcription factor that induces the expression of type I interferons (IFNs) and virus-responsive genes that control infection. Venkataraman, T., et al. (2007) Loss of DExD/H box RNA helicase LGP2 manifests disparate antiviral responses, J Immunol 178, 6444-6455. Compounds that trigger RIG-I signaling directly or through modulation of RIG-I pathway components, including IRF-3, present attractive therapeutic applications as antivirals and immune modulators.

A high-throughput screening approach was used to identify compounds that modulate the RIG-I pathway. In particular embodiments, validated RIG-I agonist lead compounds were demonstrated to specifically activate IRF-3. In additional embodiments, they have one or more of the following advantages: they induce expression of interferon-stimulated genes (ISGs), they have low cytotoxicity in cell-based assays, they are suitable for analog development and QSAR studies, they have drug-like physiochemical properties, and/or they have antiviral activity against viruses including influenza A virus, respiratory syncytial virus (RSV), and/or hepatitis C virus (HCV). In certain embodiments, the compounds exhibit all of these characteristics.

The disclosed compounds represent a new class of antiviral therapeutics. Although the disclosure is not bound by a specific mechanism of action of the compounds in vivo, the compounds are selected for their modulation of the RIG-I pathway. In certain embodiments, the modulation is activation of the RIG-I pathway. Compounds, pharmaceutical compositions, and methods disclosed herein function to treat subjects, decrease viral protein, decrease viral RNA, and/or decrease infectious virus in laboratory models of viral infection.

### I. Compounds

In one embodiment, the compounds described herein are antiviral compounds. In another embodiment, the compounds are innate immune modulating compounds. In another embodiment, the compounds are innate immune activating compounds. In another embodiment, the compounds are innate immune agonists.

In one embodiment, the compounds of the present disclosure have the structure:

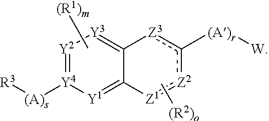

According to certain embodiments the compound may have a substitution pattern wherein the groups are as defined herein. According to specific embodiments, the compound may have a structure wherein R¹ and R² may each independently be selected from H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, haloalkyl, NH₂, OH, CN, NO₂, OCF₃, CF₃, Br, Cl, F, 1-amidino, 2-amidino, alkylcarbonyl, morpholino, piperidyl, N-alkyl piperizinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR⁴, SOR⁴, SO₂R⁴, CO₂R⁴, COR⁴, CONR⁴R⁵, CH₂CONR⁴R⁵, NR⁴SO₂R⁵, CSNR⁴R⁵ or SOₘNR⁴R⁵. R³ may be H, alkylsulfonyl, NR⁴SO₂R⁵, SOₘNR⁴R⁵, SO₂CH₃, CF₂H, CF₃, CONHCH₃, 3-propynyl, lower alkyl, aryl, alkenyl, alkynyl, haloalkyl, alkylaryl, arylalkyl, alkoxyalkylaryl, alkylamino, arylamino, heteroalkyl, heteroaryl, cyclic heteroalkyl, acyl, arylsulfonyl, heterocyclicalkylalkyl, N-imidazolinyl, N-malemido, or may be any of the groups set forth for R¹ or R². For the various embodiments of R¹, R², and R³, groups may have the following structure for R⁴ and R⁵ may each be independently selected from H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidyl, N-alkyl piperizinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, or isoquinoline. A and A′ are optional linker groups between the core bicyclic ring structure and the substituent R³ or W, respectively. That is, A and/or A′ may each be present or absent depending on the particular embodiment of the compound as shown by the value for s and r, i.e., when s or r is 1 then the respective group A or A′ is present and when s or r is 0 then the respective group A or A′ is absent. In certain embodiments, A and A′ may each independently be selected from O, S, or NR′, where R′ is H, lower alkyl or any of the groups shown for R³. According to other embodiments R′ and R³ or R′ and W may come together to form an unsubstituted or substituted heterocyclic ring or heteroaryl ring. W may be a group selected from aryl, substituted aryl, heteroaryl, substituted heteroaryl, alkyl, substituted alkyl, cycloalkyl, substituted cycloalkyl, heteroalkyl, substituted heteroalkyl, heterocycloalkyl, substituted heterocycloalkyl, arylalkyl, or heteroaryl alkyl, as defined herein. Z¹, Z², and Z³ may each independently be selected from C, O, NH, S, C═O, S═O, or SO₂. According to certain embodiments, Z¹ may be O, Z² may be C (connected to the adjacent carbon by either a single or double bond) and Z³ may be C═O. Y¹, Y², Y³, and Y⁴ may each independently be selected from C or N, provided that when Y⁴ is N, then R³-(A)ₛ is not present. For example, in certain embodiments, Y¹, Y², Y³, and Y⁴ may each be carbon, thereby forming a phenyl ring. In other embodiments, one or more of Y¹, Y², Y³, and Y⁴ may be an N. As will be understood, when Y⁴ is N, then the valence of the nitrogen is filled and the group R³-(A)ₛ will not be present. According to various embodiments, the dashed lines represent the presence or absence of a double bond. That is, the two atoms connected by the combination of a solid line and a dashed line is understood to be connected be either a single bond (sigma bond) or by a double bond (formed from the combination of a sigma bond and a pi bond). For the various substituents represented in these embodiments, the structure may have the following integer values wherein: m may be 1 or 2; n may be 0, 1, 2, or 3; o may be 0, 1, 2, or 3; s may be O or 1; and r may be 0 or 1. As will be understood by one of skill in the art, while various combinations of substituents are possible, only those combinations that are chemically compatible are within the scope of the various embodiments of the compounds of the present disclosure.

In one embodiment, one R¹ and R³ are taken together to form an aryl, cycloalkyl, methylenedioxo, ethylenedioxo, heteroaryl, or heterocycloalkyl group.

In an embodiment, R⁴ and R⁵ come together to form a morpholino ring or an N-methyl piperazinyl ring.

In another embodiment the compound has the structure:

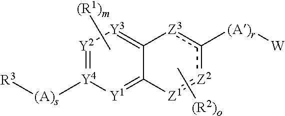

where the substituents on the ring structures can include a groups wherein s may be 1, A may be O and R³ may be H; 3-propynyl; SO₂CH₃; CF₂H; CF₃; CONHCH₃ or CH₂CONR⁴R⁵, where R⁴ and R⁵ come together to form a morpholino ring or an N-methyl piperazinyl ring; or alternatively where s may be O and R³ may be SO₂CH₃, COR⁴, CONR⁴R⁵, N-imidazolinyl or N-maleimido; and wherein r can be 0 and W can be 1-naphthyl, cyclopentyl, 2-thiazolyl, 2-pyrazinyl, 2-benzoxazolyl, or 4-R⁶-1-phenyl and R⁶ is tert-butyl, Br, OCF₃ or —NHSO₂R⁷, where R⁷ is N-piperidyl or phenyl; or alternatively, where r can be 1, and W can be phenyl.

Other example compounds have the structures:

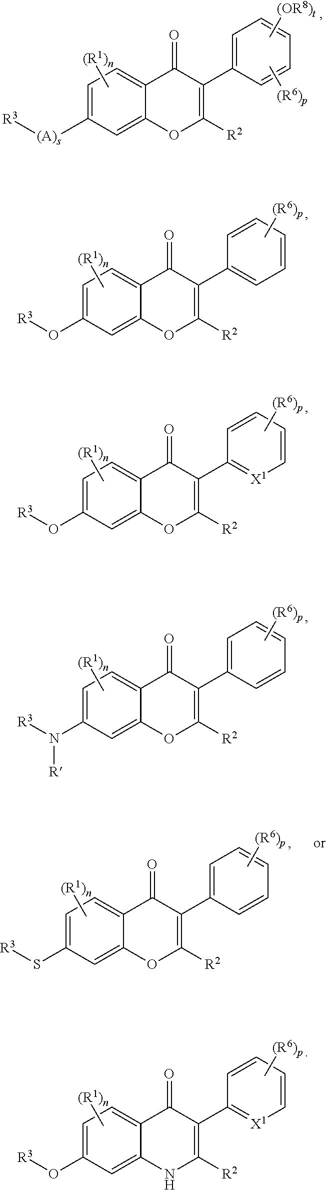

According to specific embodiments, the compounds of the present disclosure can have the structure:

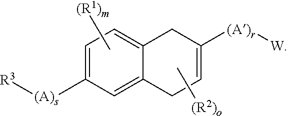

That is, according to these embodiments, the group Z¹ is O, Z² is C (connected to the adjacent carbon by either a double bond) and Z³ is be C═O; Y¹, Y², Y³ and Y⁴ are each carbon, thereby forming a phenyl ring fused to the ring containing the Z atoms.

According to other embodiments the compounds of the present disclosure can have a structure where Y⁴ is N and the compounds can have a structure:

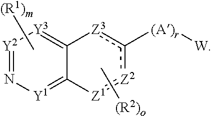

According to certain embodiments where Y⁴ is N, the compounds can have a structure:

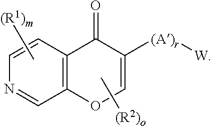

In specific embodiments, the W group can have a structure selected from:

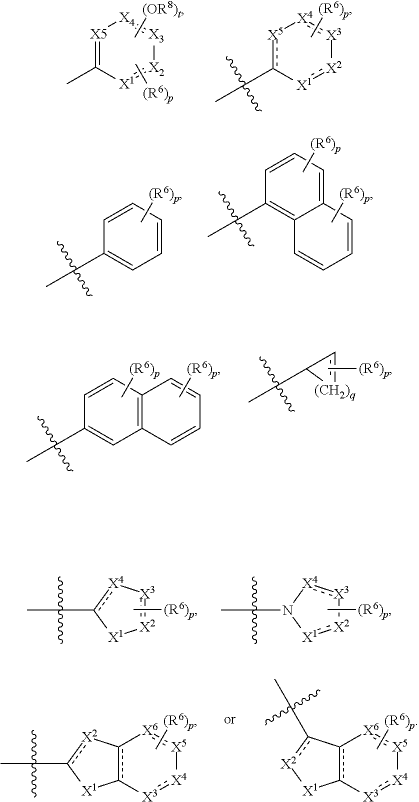

According to various embodiments of the W groups, the groups may have a structure shown herein wherein each of X¹, X², X³, X⁴, X⁵, and X⁶ may independently be selected from C, O, NH, NR⁶, S, C═O, S═O, or SO₂. Thus, the W group may typically be a substituted or unsubstituted carbocyclic, heterocyclic, aryl, or heteroaryl structure according to the structural features represented above. According to certain embodiments, the structure of W may include a substituted or unsubstituted six-membered heterocyclic ring, a carbocyclic ring, phenyl ring, or heteroaryl ring. According to other embodiments, the structure of W may include a substituted or unsubstituted naphthyl ring. Other fused aromatic and non-aromatic polycyclic ring systems are also possible for the structure of W and are within the scope of the present disclosure. In certain embodiments, the structure of W may include a substituted or unsubstituted carbocyclic ring having between 3 to 6-ring atoms (i.e., where q may be 1, 2, 3, or 4), optionally with one or more double bonds within the ring, or alternatively W may include a substituted or unsubstituted heterocyclic ring having between 3 to 7 ring atoms where one or more of the ring atoms may independently be selected from O, NH, NR⁶, S, C═O, S═O, or SO₂. In some embodiments, W can be 1-naphthyl, cyclopentyl, 2-thiazolyl, 2-pyrazinyl, 2-benzoxazolyl, or 4-R⁶-1-phenyl. According to certain embodiments where the W group is substituted, the W group may be substituted by one or more R⁶ and/or R⁸ groups, wherein one or more H atom on the W group is replaced with an R⁶ or R⁸ group. The W group may have a plurality of independently selected R⁶ and/or R⁸ groups, wherein one or up to all H atoms on the W group are replaced with an R⁶ or R⁸ substituent. According to those embodiments including a W group having one or more R⁶ substituents, each R⁶ may be independently selected from H, methyl, ethyl, propyl, isopropyl, butyl, sec-butyl, isobutyl, tert-butyl, lower alkyl, haloalkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cycloalkyl, cyclic heteroalkyl, acyl, NH₂, OH, CN, NO₂, OCF₃, CF₃, Br, Cl, F, —NHSO₂R⁷, 1-amidino, 2-amidino, alkylcarbonyl, morpholino, piperidyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, N-alkyl piperazinyl, quinoline, isoquinoline, SR⁴, SOR⁴, SO₂R⁴, CO₂R⁴, COR⁴, CONR⁴R⁵, NR⁴SO₂R⁵, CSNR⁴R⁵, or SOₘNR⁴R⁵. In an embodiment, R⁷ is alkyl, cycloalkyl, heterocycloalkyl, phenyl, aryl, heteroaryl, N-piperidyl, N-morpholino, N-alkyl-N-piperazinyl, N-pyrrolidyl, N-pyrrolidinyl, or phenyl. In certain embodiments where a W ring atom has at least two open valences (i.e., may have two substituent groups attached to the ring atom), the R⁶ group may include an unsaturated group, such as, for example ═O, ═NR⁶, ═S, or the like. In certain embodiments, the W group may include a polycyclic structure, for example where two adjacent R⁶ groups may come together to form a fused 5- or 6-membered cycloalkyl ring, heterocycloalkyl ring, methylene dioxo ring, ethylene dioxo ring, aryl ring, or heteroaryl ring. In those embodiments where two adjacent R⁶ groups come together to form a fused 5- or 6-membered ring, the fused ring may include one or more additional R⁶ substituents located on the formed fused ring off of the W ring structure. According to certain embodiments of the substituted W groups described herein the W groups may have from 0 to 5 R⁶ substituent groups, wherein each p may independently be 0, 1, 2, 3, 4, or 5; and in those embodiments including a cycloalkyl ring the q may be 1, 2, 3, or 4.

According to those embodiments including a W group having one or more R⁸ substituents, each R⁸ is independently selected from H, alkyl, haloalkyl, cycloalkyl, aryl, alkenyl, alkynyl, alkylaryl, arylalkyl, alkoxyalkylaryl, heteroalkyl, heteroaryl, cyclic heteroalkyl, acyl, CF₃, alkylcarbonyl, tetrazolo, thiazole, isothiazolo, 13uinolone, thiadiazole, thiadiazole S-oxide, thiadiazole S,S-dioxide, pyrazolo, oxazole, isoxazole, pyridinyl, pyrimidinyl, 13uinolone, isoquinoline, CO₂R⁴, COR⁴, CONR⁴R⁵, SO₂CH₃, or two adjacent R⁸ groups can come together to form a fused 5- or 6-membered cycloalkyl ring, heterocycloalkyl ring, methylene dioxo ring, ethylene dioxo ring, aryl ring or heteroaryl ring. According to certain embodiments of the substituted W groups described herein the W groups may have from 0 to 5 substituent groups selected from any of R⁶ and R⁸, wherein p and t may each independently be 0, 1, 2, 3, 4, or 5, so that p+t≦5; and in those embodiments including a cycloalkyl ring the q may be 1, 2, 3, or 4.

According to specific embodiments of the compounds of the present disclosure, the compound may have a structure where r is 0 and W is 1-naphthyl, cyclopentyl, 2-thiazolyl, 2-pyrazinyl, 2-benzoxazolyl, or 4-R⁶-1-phenyl and R⁶ is tert-butyl, Br, OCF₃, or —NHSO₂R⁷, where R⁷ is N-piperidyl or phenyl. According to other embodiments of the compounds of the present disclosure, the compound may have a structure where r is 0 and W is 4-(OR⁸)-1-phenyl and (OR⁸) is trifluoromethoxy, butanyloxy, cyclopropylmethoxy, dimethylpropoxy, trifluoroethoxy, difluoromethoxy, oxanylmethoxy, oxanylmethoxy, or dimethylbutoxy. According to still other embodiments of the compounds of the present disclosure, the compound may have a structure where r is 1, and W is phenyl.

Example compounds include wherein r is 0 and W is 1-naphthyl, cyclopentyl, 2-thiazolyl, 2-pyrazinyl, 2-benzoxazolyl, or 4-R⁶-1-phenyl and R⁶ is tert-butyl, Br, OCF₃, or —NHSO₂R⁷, where R⁷ is N-piperidyl or phenyl; or r is 1, and W is phenyl.

Other example compounds include wherein s is 1, A is O and R³ is H, 3-propynyl, SO₂CH₃, CF₂H, CF₃, CONHCH₃, C₂H₄NR⁴R⁵, or CH₂CONR⁴R⁵; where R⁴ and R⁵ come together to form a morpholino ring or an N-substituted piperazinyl ring; or s is 0 and R³ is SO₂CH₃, COR⁴, CONR⁴R⁵, N-imidazolinyl, or N-maleimido. In addition, at times, for any one compound, r is 0 and W is 1-naphthyl, cyclopentyl, 2-thiazolyl, 2-pyrazinyl, 2-benzoxazolyl, or 4-R⁶-1-phenyl and R⁶ is tert-butyl, Br, OCF₃, or —NHSO₂R⁷, where R⁷ is N-piperidyl or phenyl; or r is 1, and W is phenyl.

Other example compounds include wherein s is 1, A is NR′ where R′ is H, methyl, or ethyl; R³ is H, 3-propynyl, SO₂CH₃, CF₂H, CF₃, CONHCH₃, C₂H₄NR⁴R⁵, or CH₂CONR⁴R⁵; where R⁴ and R⁵ come together to form a morpholino ring, an N-acetyl piperazinyl ring, an N-methanesulfonyl piperazinyl ring, or an N-methyl piperazinyl ring; or s is 0 and R³ is SO₂CH₃, COR⁴, CONR⁴R⁵, N-imidazolinyl, or N-maleimido. In addition, at times, for any one compound, r is 0 and W is 1-naphthyl, cyclopentyl, 2-thiazolyl, 2-pyrazinyl, 2-benzoxazolyl, or 4-R⁶-1-phenyl and R⁶ is tert-butyl, Br, OCF₃, or —NHSO₂R⁷, where R⁷ is N-piperidyl or phenyl; or r is 1, and W is phenyl.

Other example compounds include wherein r is 0 and W is 4-(OR⁸)-1-phenyl and (OR⁸) is trifluoromethoxy, butanyloxy, cyclopropylmethoxy, dimethylpropoxy, trifluoroethoxy, difluoromethoxy, oxanylmethoxy, oxanylmethoxy, or dimethylbutoxy.

In still other embodiments of the compounds described herein, the R³ group may have a structure selected from H; 3-propynyl; SO₂CH₃; CF₂H; CF₃; CONHCH₃; COR⁴; N-imidazolinyl; N-maleimido; or CONR⁴R⁵ or CH₂CONR⁴R⁵, where R⁴ is as previously described or R⁴ and R⁵ come together to form a morpholino ring or an N-alkyl piperazinyl ring. In specific embodiments, the compound may include a compound having a structure where s is 1, A is O and R³ is H; 3-propynyl; SO₂CH₃; CF₂H; CF₃; CONHCH₃ or CH₂CONR⁴R⁵, where R⁴ and R⁵ come together to form a morpholino ring or an N-methyl piperazinyl ring. According to other embodiments, the compound may include a compound having a structure where s is 0 and R³ is SO₂CH₃, COR⁴, CONR⁴R⁵, N-imidazolinyl, or N-maleimido.

In specific embodiments, the compound described herein may include a structure where s is 1, A is O and R³ is H; 3-propynyl; SO₂CH₃; CF₂H; CF₃; CONHCH₃ or CH₂CONR⁴R⁵, where R⁴ and R⁵ come together to form a morpholino ring or an N-methyl piperazinyl ring; or alternatively s is 0 and R³ is SO₂CH₃, COR⁴, CONR⁴R⁵, N-imidazolinyl, or N-maleimido; and wherein r is 0 and W is 1-naphthyl, cyclopentyl, 2-thiazolyl, 2-pyrazinyl, 2-benzoxazolyl, or 4-R⁶-1-phenyl and R⁶ is tert-butyl, Br, OCF₃, or —NHSO₂R⁷, where R⁷ is N-piperidyl or phenyl; or alternatively r is 1, and W is phenyl.

Example compounds include R⁶ is H, methyl, ethyl, propyl, isopropyl, butyl, sec-butyl, isobutyl, tert-butyl, Cl, Br, CF₃, OCF₃, or —NHSO₂R⁷, where R⁷ is lower alkyl, cycloalkyl, heterocycloalkyl, aryl, or heteroaryl. In many instances, R⁷ is N-piperidyl, N-morpholino, N-alkyl-N-piperazinyl, or phenyl.

In particular embodiments, the compound described herein can have the structure:

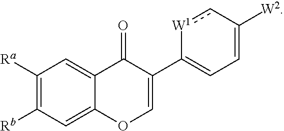

In an embodiment, W¹ can be CH, CH₂, N, or NH and W² can be Br, Cl, F, phenyl, CF₃, lower alkyl, heteroaryl, cycloalkyl, OW^{a}, C(CH₃)₃, OCH₂W^{a}, or OCH₂W^{b}, NHSO₂W^{b} or NW^{c}SO₂W^{c}. W^{b} can be Br, aryl, CF₃, lower alkyl, cycloalkyl, heterocycloalkyl, CHF₂, C(CH₃)₃, NHSO₂W^{b}; W^{b} can be phenyl, cycloalkyl, heterocycloalkyl, or lower alkyl; and W^{c} can be lower alkyl. Further, R^{a} can be H, lower alkyl or OR^{c}, where R^{c} is H or lower alkyl and Rb can be phenyl, phenol, OR^{d}, NR^{d}, OR^{d}R^{e}, or NR^{d}R^{e}. In some embodiments, Rd is lower alkyl, alkylsulfonyl, SO₂CH₃, alkylcarbonyl, CF₂, C(═O)NHR^{c}, CH₂C(═O)R^{f}, CH₂C(═O)R^{f}R^{g}, CH₂R^{h}, CH₂CH₂R^{f}, CH₂CH₂R^{f}R^{g}, CH₂CH₂R^{f}Rⁱ, where R^{e} can be hydroxyl, lower alkyl, alkylsulfonyl, or NHR^{c}. In an embodiment, R^{f} can be heteroaryl or heterocycloalkyl; R^{g} can be alkylcarbonyl, alkylsulfonyl, or lower alkyl; and R^{h} can be alkynyl.

The following definitions are applicable to the description of the compounds:

Either alone or in combination, “alkyloxy” or “alkoxy” refer to a functional group including an alkyl ether group. Examples of alkoxys include methoxy, ethoxy, n-propoxy, isopropoxy, n-butoxy, iso-butoxy, sec-butoxy, tert-butoxy, and the like.

“Alkyl”, “alkenyl”, and “alkynyl” refer to substituted and unsubstituted alkyls, alkenyls, and alkynyls.

Either alone or in combination, the term “alkyl” refers to a functional group including a straight-chain or branched-chain hydrocarbon containing from 1 to 20 carbon atoms linked exclusively by single bonds and not having any cyclic structure. “Lower alkyl” refers to a functional group containing from 1 to 6 carbon atoms. An alkyl group may be optionally substituted as defined herein. Examples of alkyl groups include, methyl, ethyl, n-propyl, isopropyl, n-butyl, isobutyl, sec-butyl, tert-butyl, pentyl, iso-amyl, hexyl, heptyl, octyl, nonyl, decyl, undecyl, dodecyl tridecyl, tetradecyl, pentadecyl, hexadecyl, heptadecyl, octadecyl, nonadecyl, eicosyl, and the like.

Either alone or in combination, the term “alkenyl” refers to a function al group including a straight-chain or branched-chain hydrocarbon containing from 2 to 20 carbon atoms and having one or more carbon-carbon double bonds and not having any cyclic structure. An alkenyl group may be optionally substituted as defined herein. Examples of alkenyl groups include ethene, propene, 2-methylpropene, 1-butene, 2-butene, pentene, 1-pentene, 2-pentene, hexene, heptene, octene, nonene, decene, undecene, dodecene, tridecene, tetradecene, pentadecene, hexadecene, heptadecene, octadecenel, nonadecene, eicosene, and the like

Either alone or in combination, “alkynyl” refers to a functional group including a straight-chain or branched-chain hydrocarbon containing from 2 to 20 carbon atoms and having one or more carbon-carbon triple bonds and not having any cyclic structure. An alkynyl group may be optionally substituted as defined herein. Examples of alkynyl groups include ethynyl, propynyl, hydroxypropynyl, butynyl, butyn-1-yl, butyn-2-yl, 3-methylbutyn-1-yl, pentynyl, pentyn-1-yl, hexynyl, hexyn-2-yl, heptynyl, octynyl, nonynyl, decynyl, undecynyl, dodecynyl, tridecynyl, tetradecynyl, pentadecynyl, hexadecynyl, heptadecynyl, octadecynyl, nonadecynyl, eicosynyl, and the like.

Either alone or in combination, substituted alkyls, alkenyls, and alkynyls refer to alkyls, alkenyls, and alkynyls substituted with one to five substituents from the group including H, lower alkyl, aryl, alkenyl, alkynyl, arylalkyl, alkoxy, aryloxy, arylalkoxy, alkoxyalkylaryl, alkylamino, arylamino, NH₂, OH, CN, NO₂, OCF₃, CF₃, F, 1-amidine, 2-amidine, alkylcarbonyl, morpholinyl, piperidinyl, dioxanyl, pyranyl, heteroaryl, furanyl, thiophenyl, tetrazolo, thiazolyl, isothiazolyl, imidazolyl, thiadiazolyl, thiadiazole S-oxide, thiadiazole S,S-dioxide, pyrazolo, oxazolyl, isoxazolyl, pyridinyl, pyrimidinyl, quinolinyl, isoquinolinyl, SR, SOR, SO₂R, CO2R, COR, CONR′R″, CSNR′R″, or SOnNR′R″ where R′ and R″ may independently be, for example, R⁴ and R⁵.

“Alkylene,” alone or in combination, refers to a saturated aliphatic group derived from a straight or branched chain saturated hydrocarbon attached at two or more positions, such as methylene (—CH₂—). Unless otherwise specified, the term “alkyl” may include “alkylene” groups.

Either alone or in combination, “alkylcarbonyl” or “alkanoyl” refer to a functional group including an alkyl group attached to the parent molecular moiety through a carbonyl group. Examples of alkylcarbonyl groups include, methylcarbonyl, ethylcarbonyl, and the like.

Either alone or in combination, “alkynylene” refers to a carbon-carbon triple bond attached at two positions, such as ethynylene (—C:::C—, —C≡C—). Unless otherwise specified, the term “alkynyl” can include “alkynylene” groups.

Either alone or in combination, “aryl”, “hydrocarbyl aryl”, or “aryl hydrocarbon” refer to a functional group including a substituted or unsubstituted aromatic hydrocarbon with a conjugated cyclic molecular ring structure of 3 to 12 carbon atoms. An aryl group can be monocyclic, bicyclic, or polycyclic, and can optionally include one to three additional ring structures, such as, e.g., a cycloalkyl, a cycloalkenyl, a heterocycloalkyl, a heterocycloalkenyl, or a heteroaryl. The term “aryl” includes phenyl (benzenyl), thiophenyl, indolyl, naphthyl, totyl, xylyl, anthracenyl, phenanthryl, azulenyl, biphenyl, naphthalenyl, 1-Methylnaphthalenyl, acenaphthenyl, acenaphthylenyl, anthracenyl, fluorenyl, phenalenyl, phenanthrenyl, benzo[a]anthracenyl, benzo[c]phenanthrenyl, chrysenyl, fluoranthenyl, pyrenyl, tetracenyl (naphthacenyl), triphenylenyl, anthanthrenyl, benzopyrenyl, benzo[a]pyrenyl, benzo[e]fluoranthenyl, benzo[ghi]perylenyl, benzo[j]fluoranthenyl, benzo[k]fluoranthenyl, corannulenyl, coronenyl, dicoronylenyl, helicenyl, heptacenyl, hexacenyl, ovalenyl, pentacenyl, picenyl, perylenyl, and tetraphenylenyl. Substituted aryl refers to aryls substituted with one to five substituents from the group including H, lower alkyl, aryl, alkenyl, alkynyl, arylalkyl, alkoxy, aryloxy, arylalkoxy, alkoxyalkylaryl, alkylamino, arylamino, NH₂, OH, CN, NO₂, OCF₃, CF₃, Br, Cl, F, 1-amidino, 2-amidino, alkylcarbonyl, morpholino, piperidinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR, SOR, SO₂R, CO₂R, COR, CONRR, CSNRR, and SOmNRR, where each R may independently be, for example, selected from R⁴ or R⁵.

Either alone or in combination, “carboxyl” or “carboxy” refers to the functional group —C(═O)OH or the corresponding “carboxylate” anion C(═O)O—. Examples include formic acid, acetic acid, oxalic acid, and benzoic acid. An “O-carboxyl” group refers to a carboxyl group having the general formula RCOO, wherein R is an organic moiety or group. A “C-carboxyl” group refers to a carboxyl group having the general formula COOR, wherein R is an organic moiety or group.

Either alone or in combination, “cycloalkyl”, “carbocyclicalkyl”, and “carbocyclealkyl” refer to a functional group including a substituted or unsubstituted non-aromatic hydrocarbon with a non-conjugated cyclic molecular ring structure of 3 to 12 carbon atoms linked exclusively with carbon-carbon single bonds in the carbon ring structure. A cycloalkyl group can be monocyclic, bicyclic, or polycyclic, and may optionally include one to three additional ring structures, such as, e.g., an aryl, a heteroaryl, a cycloalkenyl, a heterocycloalkyl, or a heterocycloalkenyl.

Either alone or in combination, “lower cycloalkyl” refers to a functional group including a monocyclic substituted or unsubstituted non-aromatic hydrocarbon with a non-conjugated cyclic molecular ring structure of 3 to 6 carbon atoms linked exclusively with carbon-carbon single bonds in the carbon ring structure. Examples of lower cycloalkyl groups include cyclopropyl, cyclobutyl, cyclopentyl, and cyclohexyl.

Either alone or in combination, “heteroalkyl” refers to a functional group including a straight-chain or branched-chain hydrocarbon containing from 1 to 20 atoms linked exclusively by single bonds, where at least one atom in the chain is a carbon and at least one atom in the chain is O, S, N, or any combination thereof. The heteroalkyl group can be fully saturated or contain from 1 to 3 degrees of unsaturation. The non-carbon atoms can be at any interior position of the heteroalkyl group, and up to two non-carbon atoms may be consecutive, such as, e.g., —CH2-NH—OCH₃. In addition, the non-carbon atoms may optionally be oxidized and the nitrogen may optionally be quaternized.

Either alone or in combination, “heteroaryl” refers to a functional group including a substituted or unsubstituted aromatic hydrocarbon with a conjugated cyclic molecular ring structure of 3 to 12 atoms, where at least one atom in the ring structure is a carbon and at least one atom in the ring structure is O, S, N, or any combination thereof. A heteroaryl group can be monocyclic, bicyclic, or polycyclic, and may optionally include one to three additional ring structures, such as, e.g., an aryl, a cycloalkyl, a cycloalkenyl, a heterocycloalkyl, or a heterocycloalkenyl. Examples of heteroaryl groups include acridinyl, benzidolyl, benzimidazolyl, benzisoxazolyl, benzodioxinyl, dihydrobenzodioxinyl, benzodioxolyl, 1,3-benzodioxolyl, benzofuryl, benzoisoxazolyl, benzopyranyl, benzothiophenyl, benzo[c]thiophenyl, benzotriazolyl, benzoxadiazolyl, benzoxazolyl, benzothiadiazolyl, benzothiazolyl, benzothienyl, carbazolyl, chromonyl, cinnolinyl, dihydrocinnolinyl, coumarinyl, dibenzofuranyl, furopyridinyl, furyl, indolizinyl, indolyl, dihydroindolyl, imidazolyl, indazolyl, isobenzofuryl, isoindolyl, isoindolinyl, dihydroisoindolyl, isoquinolyl, dihydroisoquinolinyl, isoxazolyl, isothiazolyl, oxazolyl, oxadiazolyl, phenanthrolinyl, phenanthridinyl, purinyl, pyranyl, pyrazinyl, pyrazolyl, pyridyl, pyrimidinyl, pyridazinyl, pyrrolinyl, pyrrolyl, pyrrolopyridinyl, quinolyl, quinoxalinyl, quinazolinyl, tetrahydroquinolinyl, tetrazolopyridazinyl, tetrahydroisoquinolinyl, thiophenyl, thiazolyl, thiadiazolyl, thienopyridinyl, thienyl, thiophenyl, triazolyl, xanthenyl, and the like.

Either alone or in combination, “hydroxy” refers to the functional group hydroxyl (—OH).

Either alone or in combination, “oxo” refers to the functional group ═O.

“Functional group” refers to an atom or a group of atoms that have similar chemical properties whenever they occur in different compounds, and as such the functional group defines the characteristic physical and chemical properties of families of organic compounds.

Unless otherwise indicated, when any compound or chemical structural feature, such as, for example, alkyl, aryl, etc., is referred to as being “optionally substituted,” that compound can have no substituents (in which case it is “unsubstituted”), or it can include one or more substituents (in which case it is “substituted”). The term “substituent” has the ordinary meaning known to one of ordinary skill in the art. In some embodiments, the substituent may be an ordinary organic moiety known in the art, which can have a molecular weight (e.g., the sum of the atomic masses of the atoms of the substituent) of 15 g/mol to 50 g/mol, 15 g/mol to 100 g/mol, 15 g/mol to 150 g/mol, 15 g/mol to 200 g/mol, 15 g/mol to 300 g/mol, or 15 g/mol to 500 g/mol. In some embodiments, the substituent includes: 0-30, 0-20, 0-10, or 0-5 C atoms; and/or 0-30, 0-20, 0-10, or 0-5 heteroatoms including N, O, S, Si, F, Cl, Br, or I; provided that the substituent includes at least one atom, including C, N, O, S, Si, F, Cl, Br, or I, in a substituted compound. Examples of substituents include alkyl, alkenyl, alkynyl, heteroalkyl, heteroalkenyl, heteroalkynyl, aryl, heteroaryl, hydroxy, alkoxy, aryloxy, acyl, acyloxy, alkylcarboxylate, thiol, alkylthio, cyano, halo, thiocarbonyl, O-carbamyl, N carbamyl, O thiocarbamyl, N thiocarbamyl, C amido, N amido, S-sulfonamido, N sulfonamido, isocyanato, thiocyanato, isothiocyanato, nitro, silyl, sulfenyl, sulfinyl, sulfonyl, haloalkyl, haloalkoxyl, trihalomethanesulfonyl, trihalomethanesulfonamido, amino, etc.

For convenience, the term “molecular weight” is used with respect to a moiety or part of a compound to indicate the sum of the atomic masses of the atoms in the moiety or part of a compound, even though it may not be a complete compound.

Specific embodiments of the compounds disclosed herein have the structures shown in Table 1.

TABLE 1
Select compounds of the disclosure.

| | |
|---|---|
| 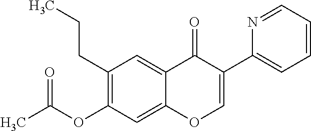 [SMILES: CCCc1cc2c(=O)c(-c3ccccn3)coc2cc1OC(C)=O] | KIN100 |
| 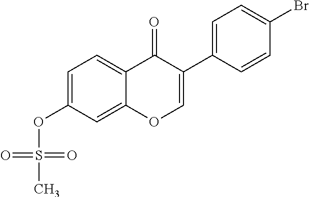 [SMILES: CS(=O)(=O)Oc1ccc2c(=O)c(-c3ccc(Br)cc3)coc2c1] | KIN101 |
| 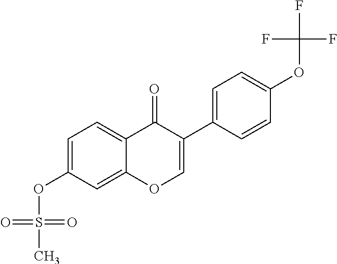 [SMILES: CS(=O)(=O)Oc1ccc2c(=O)c(-c3ccc(OC(F)(F)F)cc3)coc2c1] | KIN134 |
| 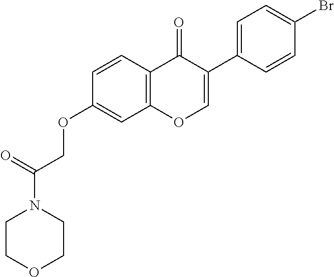 [SMILES: O=C(COc1ccc2c(=O)c(-c3ccc(Br)cc3)coc2c1)N1CCOCC1] | KIN238 |
| 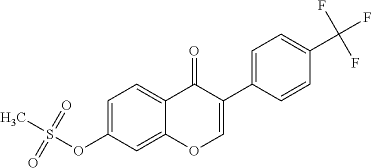 [SMILES: CS(=O)(=O)Oc1ccc2c(=O)c(-c3ccc(C(F)(F)F)cc3)coc2c1] | KIN263 |
| 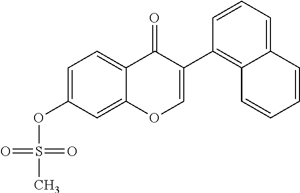 [SMILES: CS(=O)(=O)Oc1ccc2c(=O)c(-c3cccc4ccccc34)coc2c1] | KIN267 |
| 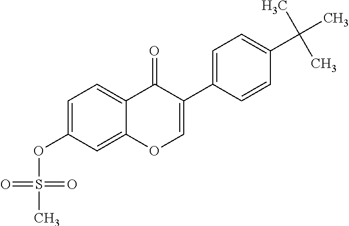 [SMILES: CC(C)(C)c1ccc(-c2coc3cc(OS(C)(=O)=O)ccc3c2=O)cc1] | KIN269 |
| 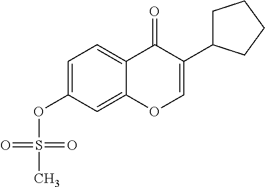 [SMILES: CS(=O)(=O)Oc1ccc2c(=O)c(C3CCCC3)coc2c1] | KIN282 |
| 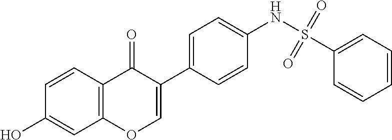 [SMILES: O=c1c(-c2ccc(NS(=O)(=O)c3ccccc3)cc2)coc2cc(O)ccc12] | KIN286 |
| 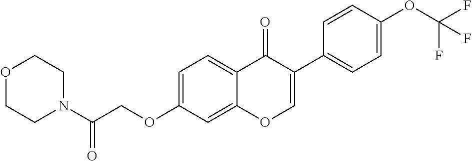 [SMILES: O=C(COc1ccc2c(=O)c(-c3ccc(OC(F)(F)F)cc3)coc2c1)N1CCOCC1] | KIN290 |
| 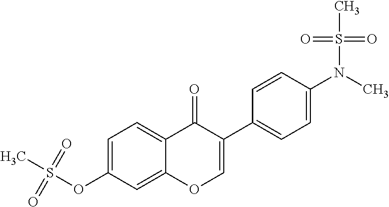 [SMILES: CN(c1ccc(-c2coc3cc(OS(C)(=O)=O)ccc3c2=O)cc1)S(C)(=O)=O] | KIN291 |
| 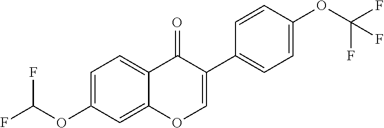 [SMILES: O=c1c(-c2ccc(OC(F)(F)F)cc2)coc2cc(OC(F)F)ccc12] | KIN299 |
| 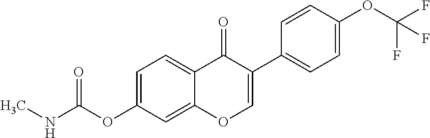 [SMILES: CNC(=O)Oc1ccc2c(=O)c(-c3ccc(OC(F)(F)F)cc3)coc2c1] | KIN302 |
| 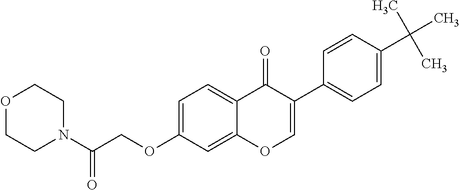 [SMILES: CC(C)(C)c1ccc(-c2coc3cc(OCC(=O)N4CCOCC4)ccc3c2=O)cc1] | KIN306 |
| 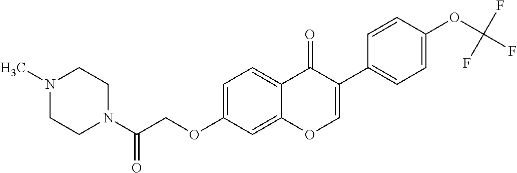 [SMILES: CN1CCN(C(=O)COc2ccc3c(=O)c(-c4ccc(OC(F)(F)F)cc4)coc3c2)CC1] | KIN307 |
| 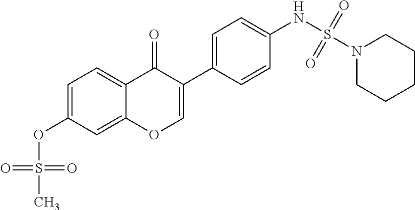 [SMILES: CS(=O)(=O)Oc1ccc2c(=O)c(-c3ccc(NS(=O)(=O)N4CCCCC4)cc3)coc2c1] | KIN308 |
| 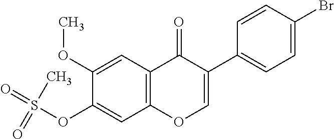 [SMILES: COc1cc2c(=O)c(-c3ccc(Br)cc3)coc2cc1OS(C)(=O)=O] | KIN320 |
| 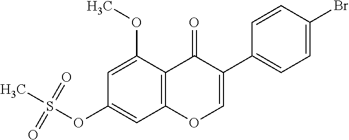 [SMILES: COc1cc(OS(C)(=O)=O)cc2occ(-c3ccc(Br)cc3)c(=O)c12] | KIN321 |
| 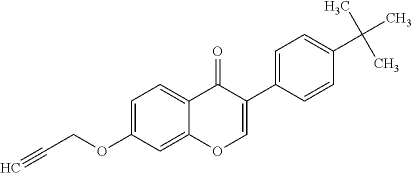 [SMILES: C#CCOc1ccc2c(=O)c(-c3ccc(C(C)(C)C)cc3)coc2c1] | KIN328 |
| 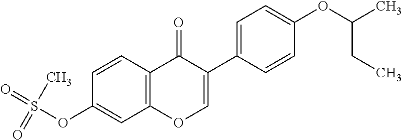 [SMILES: CCC(C)Oc1ccc(-c2coc3cc(OS(C)(=O)=O)ccc3c2=O)cc1] | KIN346 |
| 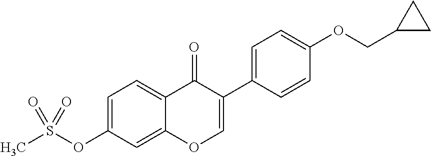 [SMILES: CS(=O)(=O)Oc1ccc2c(=O)c(-c3ccc(OCC4CC4)cc3)coc2c1] | KIN371 |
| 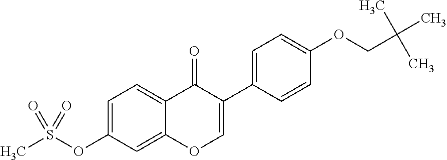 [SMILES: CC(C)(C)COc1ccc(-c2coc3cc(OS(C)(=O)=O)ccc3c2=O)cc1] | KIN372 |
| 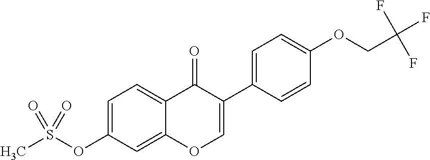 [SMILES: CS(=O)(=O)Oc1ccc2c(=O)c(-c3ccc(OCC(F)(F)F)cc3)coc2c1] | KIN376 |
| 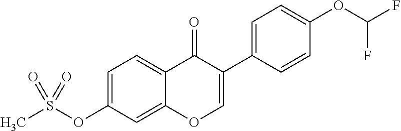 [SMILES: CS(=O)(=O)Oc1ccc2c(=O)c(-c3ccc(OC(F)F)cc3)coc2c1] | KIN378 |
| 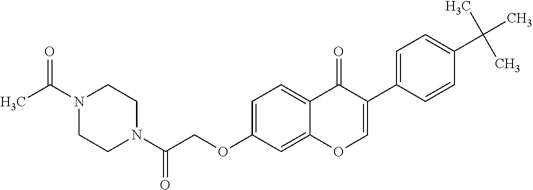 [SMILES: CC(=O)N1CCN(C(=O)COc2ccc3c(=O)c(-c4ccc(C(C)(C)C)cc4)coc3c2)CC1] | KIN380 |
| 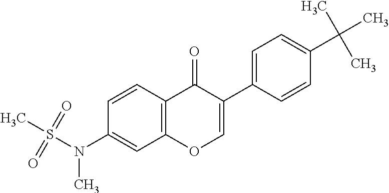 [SMILES: CN(c1ccc2c(=O)c(-c3ccc(C(C)(C)C)cc3)coc2c1)S(C)(=O)=O] | KIN385 |
| 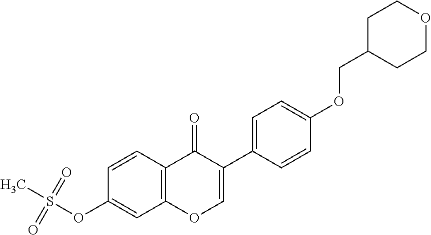 [SMILES: CS(=O)(=O)Oc1ccc2c(=O)c(-c3ccc(OCC4CCOCC4)cc3)coc2c1] | KIN389 |
| 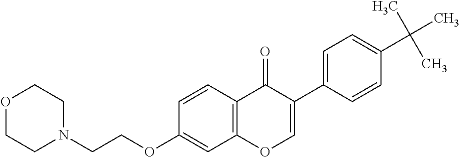 [SMILES: CC(C)(C)c1ccc(-c2coc3cc(OCCN4CCOCC4)ccc3c2=O)cc1] | KIN392 |
| 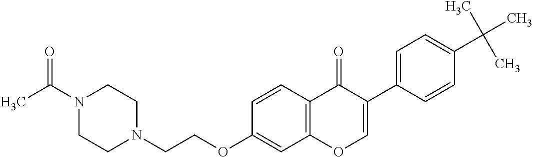 [SMILES: CC(=O)N1CCN(CCOc2ccc3c(=O)c(-c4ccc(C(C)(C)C)cc4)coc3c2)CC1] | KIN394 |
| 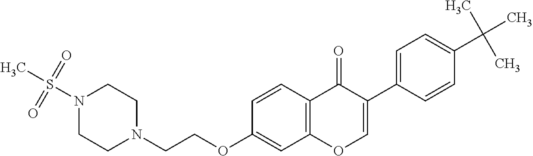 [SMILES: CC(C)(C)c1ccc(-c2coc3cc(OCCN4CCN(S(C)(=O)=O)CC4)ccc3c2=O)cc1] | KIN395 |
| 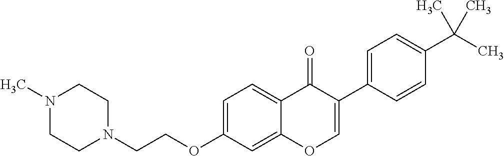 [SMILES: CN1CCN(CCOc2ccc3c(=O)c(-c4ccc(C(C)(C)C)cc4)coc3c2)CC1] | KIN807 |
| 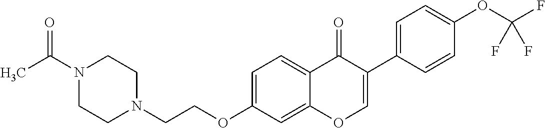 [SMILES: CC(=O)N1CCN(CCOc2ccc3c(=O)c(-c4ccc(OC(F)(F)F)cc4)coc3c2)CC1] | KIN814 |
| 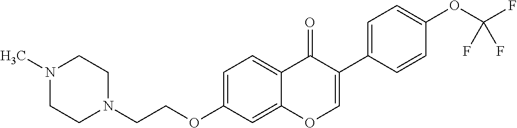 [SMILES: CN1CCN(CCOc2ccc3c(=O)c(-c4ccc(OC(F)(F)F)cc4)coc3c2)CC1] | KIN823 |
| 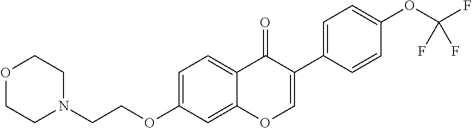 [SMILES: O=c1c(-c2ccc(OC(F)(F)F)cc2)coc2cc(OCCN3CCOCC3)ccc12] | KIN824 |
| 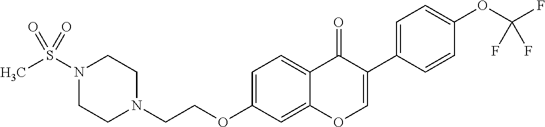 [SMILES: CS(=O)(=O)N1CCN(CCOc2ccc3c(=O)c(-c4ccc(OC(F)(F)F)cc4)coc3c2)CC1] | KIN826 |
| 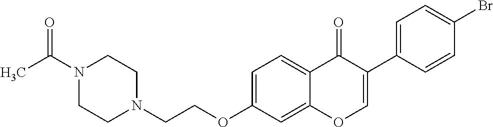 [SMILES: CC(=O)N1CCN(CCOc2ccc3c(=O)c(-c4ccc(Br)cc4)coc3c2)CC1] | KIN844 |
| 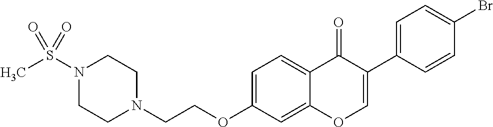 [SMILES: CS(=O)(=O)N1CCN(CCOc2ccc3c(=O)c(-c4ccc(Br)cc4)coc3c2)CC1] | KIN848 |
| 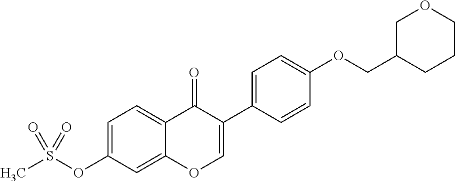 [SMILES: CS(=O)(=O)Oc1ccc2c(=O)c(-c3ccc(OCC4CCCOC4)cc3)coc2c1] | KIN850 |
| 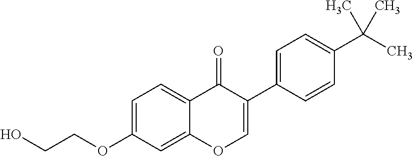 [SMILES: CC(C)(C)c1ccc(-c2coc3cc(OCCO)ccc3c2=O)cc1] | KIN851 |
| 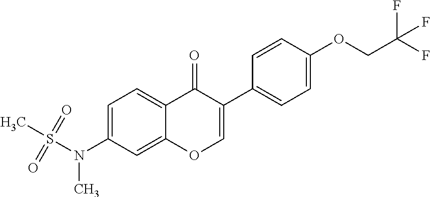 [SMILES: CN(c1ccc2c(=O)c(-c3ccc(OCC(F)(F)F)cc3)coc2c1)S(C)(=O)=O] | KIN857 |
| 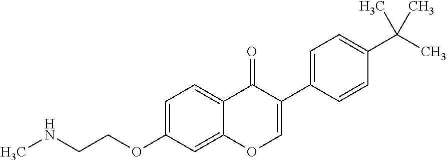 [SMILES: CNCCOc1ccc2c(=O)c(-c3ccc(C(C)(C)C)cc3)coc2c1] | KIN861 |
| 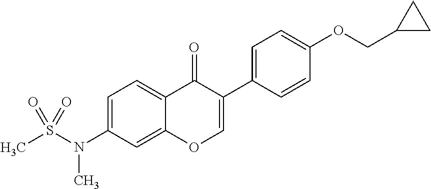 [SMILES: CN(c1ccc2c(=O)c(-c3ccc(OCC4CC4)cc3)coc2c1)S(C)(=O)=O] | KIN865 |
| 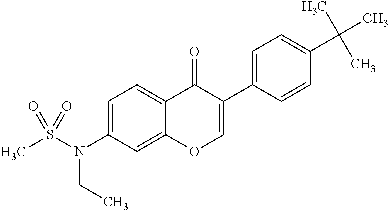 [SMILES: CCN(c1ccc2c(=O)c(-c3ccc(C(C)(C)C)cc3)coc2c1)S(C)(=O)=O] | KIN866 |
| 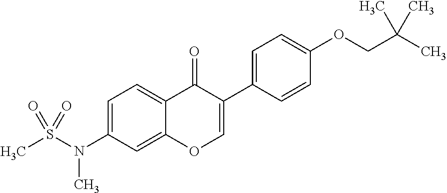 [SMILES: CN(c1ccc2c(=O)c(-c3ccc(OCC(C)(C)C)cc3)coc2c1)S(C)(=O)=O] | KIN867 |
| 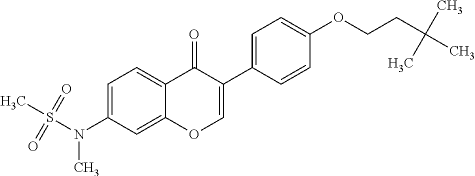 [SMILES: CN(c1ccc2c(=O)c(-c3ccc(OCCC(C)(C)C)cc3)coc2c1)S(C)(=O)=O] | KIN882 |

Unless stereochemistry is unambiguously depicted, any structure, formula, or name for a compound can refer to any stereoisomer or any mixture of stereoisomers of the compound.

Compounds can also be provided as alternate solid forms, such as polymorphs, solvates, hydrates, etc.; tautomers; or any other chemical species that may rapidly convert to a compound described herein under conditions in which the compounds are used as described herein. Compounds also include pharmaceutically acceptable salts of the compounds.

As used herein, the term “pharmaceutically acceptable salt” refers to pharmaceutical salts that are, within the scope of sound medical judgment, suitable for use in contact with the tissues of subjects without undue toxicity, irritation, and allergic response, and are commensurate with a reasonable benefit/risk ratio. Pharmaceutically acceptable salts are well known in the art. In one embodiment, the pharmaceutically acceptable salt is a sulfate salt. For example, S. M. Berge, et al. describes pharmaceutically acceptable salts in J. Pharm. Sci., 1977, 66:1-19.

Suitable pharmaceutically acceptable acid addition salts can be prepared from an inorganic acid or an organic acid. Examples of such inorganic acids are hydrochloric, hydrobromic, hydroiodic, nitric, carbonic, sulfuric and phosphoric acid. Appropriate organic acids can be selected from aliphatic, cycloaliphatic, aromatic, arylaliphatic, heterocyclic, carboxylic and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, β-hydroxybutyric, malonic, galactic, and galacturonic acid. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts.

Suitable pharmaceutically acceptable base addition salts include, but are not limited to, metallic salts made from aluminum, calcium, lithium, magnesium, potassium, sodium and zinc or organic salts made from N,N′-dibenzylethylene-diamine, chloroprocaine, choline, diethanolamine, ethylenediamine, N-methylglucamine, lysine, arginine and procaine. All of these salts can be prepared by conventional means from the corresponding compound represented by the disclosed compounds by treating, for example, the disclosed compounds with the appropriate acid or base. Pharmaceutically acceptable basic/cationic salts also include, the diethanolamine, ammonium, ethanolamine, piperazine and triethanolamine salts.

A pharmaceutically acceptable salt includes any salt that retains the activity of the parent compound and is acceptable for pharmaceutical use. A pharmaceutically acceptable salt also refers to any salt which may form in vivo as a result of administration of an acid, another salt, or a prodrug which is converted into an acid or salt.

Compounds disclosed herein also include prodrugs. A prodrug includes a compound which is converted to a therapeutically active compound after administration, such as by hydrolysis of an ester group or some other biologically labile group.

### II. Pharmaceutical Compositions

According to other embodiments, the present disclosure provides for a pharmaceutical composition including any one of the compounds described herein.

Pharmaceutical compositions can be formed by combining a compound disclosed herein, or a pharmaceutically acceptable prodrug or salt thereof, with a pharmaceutically acceptable carrier suitable for delivery to a subject in accordance with known methods of drug delivery. Accordingly, a “pharmaceutical composition” includes at least one compound disclosed herein together with one or more pharmaceutically acceptable carriers, excipients, or diluents, as appropriate for the chosen mode of administration.

The pharmaceutical composition including a compound of the disclosure can be formulated in a variety of forms depending upon the particular indication being treated and will be apparent to one of ordinary skill in the art. Formulating pharmaceutical compositions including one or more compounds of the disclosure can employ straightforward medicinal chemistry processes. The pharmaceutical compositions can be subjected to conventional pharmaceutical operations such as sterilization and/or can contain conventional adjuvants, such as buffering agents, preservatives, isotonicifiers, stabilizers, wetting agents, emulsifiers, etc.

Buffering agents help to maintain the pH in a range which approximates physiological conditions. They are typically present at a concentration ranging from 2 mM to 50 mM of a pharmaceutical composition. Suitable buffering agents include both organic and inorganic acids, and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.), and acetate buffers (e.g., acetic acid-sodium acetate mixture, acetic acid-sodium hydroxide mixture, etc.). Additional possibilities are phosphate buffers, histidine buffers, and trimethylamine salts such as Tris.

Preservatives can be added to pharmaceutical compositions to retard microbial growth, and are typically added in amounts of 0.2%-1% (w/v). Suitable preservatives include phenol, benzyl alcohol, meta-cresol, methyl paraben, propyl paraben, octadecyldimethylbenzyl ammonium chloride, benzalkonium halides (e.g., benzalkonium chloride, bromide or iodide), hexamethonium chloride, alkyl parabens such as methyl or propyl paraben, catechol, resorcinol, cyclohexanol, and 3-pentanol.

Isotonicifiers can be added to pharmaceutical compositions to ensure isotonicity. Appropriate isotonicifiers include polyhydric sugar alcohols, preferably trihydric or higher sugar alcohols, such as glycerin, erythritol, arabitol, xylitol, sorbitol, and mannitol. Polyhydric alcohols can be present in an amount between 0.1% and 25% by weight, typically 1% to 5%, taking into account the relative amounts of the other ingredients.

Stabilizers refer to a broad category of excipients which can range in function from a bulking agent to an additive which solubilizes the compound or helps to prevent denaturation or adherence to the container wall. Typical stabilizers can be polyhydric sugar alcohols; amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol, and the like, including cyclitols such as inositol; polyethylene glycol; amino acid polymers; sulfur-containing reducing agents, such as urea, glutathione, thioctic acid, sodium thioglycolate, thioglycerol, alpha-monothioglycerol and sodium thiosulfate; low molecular weight polypeptides (i.e., <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on compound weight.

Additional miscellaneous excipients can include chelating agents (e.g., EDTA), antioxidants (e.g., ascorbic acid, methionine, and vitamin E) and cosolvents.

Particular embodiments can include one or more of ethanol (<10%), propylene glycol (<40%), polyethylene glycol (PEG) 300 or 400 (<60%), N—N-dimethylacetamide (DMA, <30%), N-methyl-2-pyrrolidone (NMP, <20%), dimethyl sulfoxide (DMSO, <20%) co-solvents or the cyclodextrins (<40%) and have a pH of 3 to 9.

Generally, the pharmaceutical compositions can be made up in a solid form (including granules, powders, or suppositories) or in a liquid form (e.g., solutions, suspensions, or emulsions). The compounds can be admixed with adjuvants such as lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinyl-pyrrolidine, and/or polyvinyl alcohol, and tableted or encapsulated for conventional administration. Alternatively, they can be dissolved in saline, water, polyethylene glycol, propylene glycol, ethanol, oils (such as corn oil, peanut oil, cottonseed oil, or sesame oil), tragacanth gum, and/or various buffers. Other adjuvants and modes of administration are well known in the pharmaceutical art. The carrier or diluent can include time delay material, such as glyceryl monostearate or glyceryl distearate alone or with a wax, or other materials well known in the art.

Oral administration of the pharmaceutical compositions is one intended practice of the disclosure. For oral administration, the pharmaceutical composition can be in solid or liquid form, e.g., in the form of a capsule, tablet, powder, granule, suspension, emulsion, or solution.

Solid dosage forms for oral administration can include capsules, tablets, pills, powders, and granules. In such solid dosage forms, the compound can be admixed with at least one inert diluent such as sucrose, lactose, or starch. Such dosage forms can also include, as in normal practice, additional substances other than inert diluents, e.g., lubricating agents such as magnesium stearate. In the case of capsules, tablets, and pills, the dosage forms can also include buffering agents. Tablets and pills can additionally be prepared with enteric coatings. For buccal administration the pharmaceutical compositions can take the form of tablets or lozenges formulated in conventional manners.

Liquid dosage forms for oral administration can include pharmaceutically acceptable emulsions, solutions, suspensions, syrups, and elixirs containing inert diluents commonly used in the art, such as water. Such pharmaceutical compositions can also include adjuvants, such as wetting, sweetening, flavoring, and perfuming agents.

The pharmaceutical compositions can be formulated for parenteral administration by injection, e.g. by bolus injection, or infusion. Formulations for injection can be presented in unit dosage form, e.g. in glass ampoule or multi-dose containers, e.g. glass vials. The pharmaceutical compositions for injection can take such forms as suspensions, solutions, or emulsions in oily or aqueous vehicles, and can contain formulatory agents such as antioxidants, buffers, non-ionic detergents, dispersants, isotonicifiers, suspending agents, stabilizers, preservatives, dispersing agents and/or other miscellaneous additives. Parenteral formulations to be used for in vivo administration generally are sterile. This is readily accomplished, for example, by filtration through sterile filtration membranes.

Although in many cases pharmaceutical compositions provided in liquid form are appropriate for immediate use, such parenteral formulations can also be provided in frozen or in lyophilized form. In the former case, the pharmaceutical composition must be thawed prior to use. The latter form is often used to enhance the stability of the compound contained in the pharmaceutical composition under a wider variety of storage conditions, as it is recognized by those or ordinary skill in the art that lyophilized preparations are generally more stable than their liquid counterparts. Parenterals can be prepared for storage as lyophilized formulations by mixing, as appropriate, the compound having the desired degree of purity with one or more pharmaceutically acceptable carriers, excipients, or stabilizers typically employed in the art (all of which are termed “excipients”), for example, antioxidants, buffers, non-ionic detergents, dispersants, isotonicifiers, suspending agents, stabilizers, preservatives, dispersing agents and/or other miscellaneous additives. Such lyophilized preparations are reconstituted prior to use by the addition of one or more suitable pharmaceutically acceptable diluents such as sterile pyrogen-free water for injection or sterile physiological saline solution.

For administration by inhalation (e.g., nasal or pulmonary), the pharmaceutical compositions can be conveniently delivered in the form of an aerosol spray, from pressurized packs or a nebulizer, and/or with the use of suitable propellant, e.g. dichlorodifluoromethane, trichlorofluoromethane, dichlorotetrafluoroethane, carbon dioxide, or other suitable gases or mixture of gases.

In addition to the formulations described above, the pharmaceutical compositions can also be formulated as depot preparations. Such long acting formulations can be administered by implantation or by intramuscular injection.

The compounds can also be entrapped in microcapsules prepared, for example, by coascervation techniques or by interfacial polymerization, (for example hydroxymethylcellulose, gelatin or poly-(methylmethacylate) microcapsules), in colloidal drug delivery systems (for example liposomes, albumin microspheres, microemulsions, nano-particles and nanocapsules) or in macroemulsions. Such techniques are disclosed in Remington, The Science and Practice of Pharmacy, 21st Ed., published by Lippincott Williams & Wilkins, A Wolters Kluwer Company, 2005.

Additional suitable examples of sustained-release preparations include semi-permeable matrices of solid hydrophobic polymers containing the compound, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the PROLEASE® technology (Alkermes, Inc., Cambridge, Mass.) or LUPRON DEPOT® (Tap Pharmaceuticals Products, Inc.; Lake Forest, Ill.; injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for long periods such as up to or over 100 days, certain hydrogels release compounds for shorter time periods.

### III. Methods of Use

The pharmaceutical compositions disclosed herein can be used to treat a viral infection in a subject; wherein the viral infection is caused by a virus from one the following families: Arenaviridae, Arterivirus, Astroviridae, Birnaviridae, Bromoviridae, Bunyaviridae, Caliciviridae, Closteroviridae, Comoviridae, Coronaviridae, Cystoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Paramyxoviridae, Picobirnaviridae, Picobirnavirus, Picornaviridae, Potyviridae, Reoviridae, Retroviridae, Roniviridae, Sequiviridae, Tenuivirus, Togaviridae, Tombusviridae, Totiviridae, and Tymoviridae.

According to more specific embodiments, the pharmaceutical compositions can be used to treat a viral infection caused by one or more of Alfuy virus, Banzi virus, bovine diarrhea virus, Chikungunya virus, Dengue virus (DNV), Encephalomyocarditis virus (EMCV), Hepatitis B virus (HBV), HCV, human cytomegalovirus (hCMV), HIV, Ilheus virus, influenza virus (including avian and swine isolates), Japanese encephalitis virus, Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, WNV, and yellow fever virus.

Many RNA viruses share biochemical, regulatory, and signaling pathways. These viruses include influenza viruses (including avian and swine isolates), DNV, RSV, WNV, HCV, parainfluenza virus, metapneumovirus, Chikungunya virus, SARS, MERS, poliovirus, measles virus, yellow fever virus, tick-borne encephalitis virus, Japanese encephalitis virus, St. Louis encephalitis virus, Murray Valley virus, Powassan virus, Rocio virus, louping-ill virus, Banzi virus, Ilheus virus, Kokobera virus, Kunjin virus, Alfuy virus, bovine diarrhea virus, and the Kyasanur forest disease virus.

Methods disclosed herein include treating subjects (humans, mammals, free-range herds, veterinary animals (dogs, cats, reptiles, birds, etc.), farm animals and livestock (horses, cattle, goats, pigs, chickens, etc.), and research animals (monkeys, rats, mice, fish, etc.)) with pharmaceutical compositions disclosed herein. Treating subjects includes delivering therapeutically effective amounts. Therapeutically effective amounts include those that provide effective amounts, prophylactic treatments, and/or therapeutic treatments.

An “effective amount” is the amount of a compound necessary to result in a desired physiological change in the subject. Effective amounts are often administered for research purposes. Effective amounts disclosed herein reduce, control, or eliminate the presence or activity of viral infections and/or reduce, control, or eliminate unwanted side effects of viral infections. For example, an effective amount may result in a reduction in viral protein in a subject or assay, a reduction in viral RNA in a subject or assay, and/or a reduction in virus present in a cell culture.

A “prophylactic treatment” includes a treatment administered to a subject who does not display signs or symptoms of a viral infection or displays only early signs or symptoms of the viral infection such that treatment is administered for the purpose of diminishing, preventing, or decreasing the risk of developing the viral infection further. Thus, a prophylactic treatment functions as a preventative treatment against a viral infection. Prophylactic treatment may also include vaccines as described elsewhere herein. Prophylactic treatment may result in a lack of increase in viral proteins or RNA in a subject, and/or a lack of increase in clinical indicators of viral infection, such as: loss of appetite, fatigue, fever, muscle aches, nausea, and/or abdominal pain in the case of HCV; fever and/or headache in the case of WNV; and cough, congestion, fever, sore throat, and/or headache in the case of RSV. Prophylactic treatments can be administered to any subject regardless of whether signs of viral infection are present. In some embodiments, prophylactic treatments can be administered before travel.

A “therapeutic treatment” includes a treatment administered to a subject who displays symptoms or signs of a viral infection and is administered to the subject for the purpose of diminishing or eliminating the signs or symptoms of the viral infection. The therapeutic treatment can reduce, control, or eliminate the presence or activity of viruses and/or reduce, control, or eliminate side effects of viruses. Therapeutic treatment may result in a decrease in viral proteins or RNA in a subject, and/or a decrease in clinical indicators of viral infection, such as: loss of appetite, fatigue, fever, muscle aches, nausea, and/or abdominal pain in the case of HCV; fever and/or headache in the case of WNV; and cough, congestion, fever, cyanosis, sore throat, and/or headache in the case of RSV.

For administration, therapeutically effective amounts (also referred to herein as doses) can be initially estimated based on results from in vitro assays and/or animal model studies. For example, a dose can be formulated in animal models to achieve a circulating concentration range that includes an 1050 as determined in cell culture against a particular target. Such information can be used to more accurately determine useful doses in subjects of interest.

The actual dose amount administered to a particular subject can be determined by a physician, veterinarian, or researcher taking into account parameters such as physical and physiological factors including target, body weight, severity of condition, type of viral infection, previous or concurrent therapeutic interventions, idiopathy of the subject, and route of administration.

Pharmaceutical compositions can be administered intravenously to a subject for treatment of viral infections in a clinically safe and effective manner, including one or more separate administrations of the composition. For example, 0.05 mg/kg to 5.0 mg/kg can be administered to a subject per day in one or more doses (e.g., doses of 0.05 mg/kg once-daily (QD), 0.10 mg/kg QD, 0.50 mg/kg QD, 1.0 mg/kg QD, 1.5 mg/kg QD, 2.0 mg/kg QD, 2.5 mg/kg QD, 3.0 mg/kg QD, 0.75 mg/kg twice-daily (BID), 1.5 mg/kg BID or 2.0 mg/kg BID). For certain antiviral indications, the total daily dose of a compound can be 0.05 mg/kg to 3.0 mg/kg administered intravenously to a subject one to three times a day, including administration of total daily doses of 0.05-3.0, 0.1-3.0, 0.5-3.0, 1.0-3.0, 1.5-3.0, 2.0-3.0, 2.5-3.0, and 0.5-3.0 mg/kg/day of compounds of Table 1 using 60-minute QD, BID, or three times daily (TID) intravenous infusion dosing. In one particular example, antiviral pharmaceutical compositions can be intravenously administered QD or BID to a subject with, e.g., total daily doses of 1.5 mg/kg, 3.0 mg/kg, 4.0 mg/kg of a composition with up to 92-98% wt/wt of a compound of Table 1.

Additional useful doses can often range from 0.1 to 5 μg/kg or from 0.5 to 1 μg/kg. In other examples, a dose can include 1 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, 150 μg/kg, 200 μg/kg, 250 μg/kg, 350 μg/kg, 400 μg/kg, 450 μg/kg, 500 μg/kg, 550 μg/kg, 600 μg/kg, 650 μg/kg, 700 μg/kg, 750 μg/kg, 800 μg/kg, 850 μg/kg, 900 μg/kg, 950 μg/kg, 1000 μg/kg, 0.1 to 5 mg/kg, or from 0.5 to 1 mg/kg. In other examples, a dose can include 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, 70 mg/kg, 75 mg/kg, 80 mg/kg, 85 mg/kg, 90 mg/kg, 95 mg/kg, 100 mg/kg, 150 mg/kg, 200 mg/kg, 250 mg/kg, 350 mg/kg, 400 mg/kg, 450 mg/kg, 500 mg/kg, 550 mg/kg, 600 mg/kg, 650 mg/kg, 700 mg/kg, 750 mg/kg, 800 mg/kg, 850 mg/kg, 900 mg/kg, 950 mg/kg, 1000 mg/kg, or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months, or yearly.

The administration of the pharmaceutical compositions of the present disclosure can be performed in a variety of ways, including orally, subcutaneously, intravenously, intracerebrally, intranasally, transdermally, intraperitoneally, intramuscularly, intrapulmonary, intrathecally, vaginally, rectally, intraocularly, or in any other acceptable manner. The pharmaceutical compositions can be administered continuously by infusion, although bolus injection is acceptable, using techniques well known in the art, such as pumps (e.g., subcutaneous osmotic pumps) or implantation. In some instances the pharmaceutical compositions can be directly applied as a solution or spray.

The pharmaceutical compositions disclosed herein can be additive or synergistic with other therapies currently in development or use. For example, ribavirin and interferon-α provide an effective treatment for HCV infection when used in combination. Their efficacy in combination can exceed the efficacy of either drug product when used alone. The pharmaceutical compositions of the disclosure can be administered alone or in combination or conjunction with interferon, ribavirin, and/or a variety of small molecules that are being developed against both viral targets (viral proteases, viral polymerase, and/or assembly of viral replication complexes) and host targets (host proteases required for viral processing, host kinases required for phosphorylation of viral targets such as NS5A, and inhibitors of host factors required to efficiently utilize the viral internal ribosome entry site, or IRES).

The pharmaceutical compositions disclosed herein could be used in combination or conjunction with adamantane inhibitors, neuraminidase inhibitors, alpha interferons, non-nucleoside or nucleoside polymerase inhibitors, NS5A inhibitors, antihistamines, protease inhibitors, helicase inhibitors, P7 inhibitors, entry inhibitors, IRES inhibitors, immune stimulators, HCV replication inhibitors, cyclophilin A inhibitors, A3 adenosine agonists, and/or microRNA suppressors.

Cytokines that could be administered in combination or conjunction with the pharmaceutical compositions disclosed herein include interleukin (IL)-2, IL-12, IL-23, IL-27, or IFN-γ.

New HCV drugs that are, or will be, available for potential administration in combination or conjunction with the pharmaceutical compositions disclosed herein include ACH-1625 (Achillion); Glycosylated interferon (Alios Biopharma); ANA598, ANA773 (Anadys Pharm); ATI-0810 (Arisyn Therapeutics); AVL-181 (Avila Therapeutics); LOCTERON® (Biolex); CTS-1027 (Conatus); SD-101 (Dynavax Technologies); Clemizole (Eiger Biopharmaceuticals); GS-9190 (Gilead Sciences); GI-5005 (Globallmmune BioPharma); Resiquimod/R-848 (Graceway Pharmaceuticals); Albinterferon alpha-2b (Human Genome Sciences); IDX-184, IDX-320, IDX-375 (Idenix); IMO-2125 (Idera Pharmaceuticals); INX-189 (Inhibitex); ITCA-638 (Intarcia Therapeutics); ITMN-191/RG7227 (Intermune); ITX-5061, ITX-4520 (iTherx Pharmaceuticals); MB11362 (Metabasis Therapeutics); Bavituximab (Peregrine Pharmaceuticals); PSI-7977, RG7128, PSI-938 (Pharmasset); PHX1766 (Phenomix); Nitazoxanide/ALINIA® (Romark Laboratories); SP-30 (Samaritan Pharmaceuticals); SCV-07 (SciClone); SCY-635 (Scynexis); TT-033 (Tacere Therapeutics); Viramidine/taribavirin (Valeant Pharmaceuticals); Telaprevir, VCH-759, VCH-916, VCH-222, VX-500, VX-813 (Vertex Pharmaceuticals); and PEG-INF Lambda (Zymogenetics).

New influenza and WNV drugs that are, or will be, available for potential administration in combination or conjunction with the pharmaceutical compositions disclosed herein include neuraminidase inhibitors (Peramivir, Laninamivir); triple therapy—neuraminidase inhibitors, ribavirin, and amantadine (ADS-8902); polymerase inhibitors (Favipiravir); reverse transcriptase inhibitor (ANX-201); inhaled chitosan (ANX-211); entry/binding inhibitors (Binding Site Mimetic, Flucide); entry inhibitor, (Fludase); fusion inhibitor, (MGAWN1 for WNV); host cell inhibitors (lantibiotics); cleavage of RNA genome (RNAi, RNAse L); immune stimulators (Interferon, Alferon-LDO; Neurokinin) agonist, Homspera, Interferon Alferon N for WNV); and TG21.

Other drugs for treatment of influenza and/or hepatitis that are available for potential administration in combination or conjunction with the pharmaceutical compositions include those provided in Table 2.

TABLE 2
Hepatitis and influenza drugs

| Branded Name | Generic Name | Approved Indications |
|---|---|---|
| Pegasys | PEGinterferon alfa-2a | HCV, HBV |
| Peg-Intron | PEGinterferon alfa-2b | HCV |
| Copegus | Ribavirin | HCV |
| Rebetol | Ribavirin | HCV |
| — | Ribavirin | HCV |
| Tamiflu | Oseltamivir | Influenza A, B, C |
| Relenza | Zanamivir | Influenza A, B, C |
| — | Amantadine | Influenza A |
| — | Rimantadine | Influenza A |

The compounds or pharmaceutical compositions can be additive or synergistic with other compounds or pharmaceutical compositions to enable vaccine development. By virtue of their antiviral and immune enhancing properties, the compounds can be used to affect a prophylactic or therapeutic vaccination. The compounds need not be administered simultaneously or in combination with other vaccine components to be effective. The vaccine applications of the compounds are not limited to the treatment of viral infection but can encompass all therapeutic and prophylactic vaccine applications due to the general nature of the immune response elicited by the compounds.

A “vaccine” is an immunogenic preparation that is used to induce an immune response in an individual. A vaccine can have more than one constituent that is immunogenic. A vaccine can be used for prophylactic and/or therapeutic purposes. A vaccine does not necessarily have to prevent viral infections. Without being bound by theory, the vaccines of the disclosure can affect an individual's immune response in a manner such that viral infection occurs in a lesser amount (including not at all) or such that biological or physiological effects of the viral infection are ameliorated when the vaccine is administered as described herein. As used herein, vaccines include preparations including pharmaceutical compositions including the compounds, alone or in combination with an antigen, for the purpose of treating a viral infection in a subject including a vertebrate animal.

The disclosure provides for the use of the compounds and pharmaceutical compositions as adjuvants. An adjuvant enhances, potentiates, and/or accelerates the beneficial effects of another administered therapeutic agent. In particular embodiments, the term “adjuvant” refers to compounds that modify the effect of other agents on the immune system. Adjuvants that possess this function may also be inorganic or organic chemicals, macromolecules, or entire cells of certain killed bacteria, which enhance the immune response to an antigen. They may be included in a vaccine to enhance the recipient's immune response to the supplied antigen.

As is understood by one of ordinary skill in the art, vaccines can be against viruses, bacterial infections, cancers, etc. and can include one or more of a live attenuated vaccine (LAIV), an inactivated vaccine (IIV; killed virus vaccine), a subunit (split vaccine); a sub-virion vaccine; a purified protein vaccine; or a DNA vaccine. Appropriate adjuvants include one or more of water/oil emulsions, non-ionic copolymer adjuvants, e.g., CRL 1005 (Optivax; Vaxcel Inc., Norcross, Ga.), aluminum phosphate, aluminum hydroxide, aqueous suspensions of aluminum and magnesium hydroxides, bacterial endotoxins, polynucleotides, polyelectrolytes, lipophilic adjuvants and synthetic muramyl dipeptide (norMDP) analogs such as N-acetyl-nor-muranyl-L-alanyl-D-isoglutamine, N-acetyl-muranyl-(6-O-stearoyl)-L-alanyl-D-isoglutamine, or N-Glycol-muranyl-LalphaAbu-D-isoglutamine (Ciba-Geigy Ltd.).

The present disclosure further includes the use and application of the compounds and pharmaceutical compositions in vitro in a number of applications including developing therapies and vaccines against viral infections, research in modulation of the innate immune response in eukaryotic cells, etc. The compounds and pharmaceutical compositions disclosure can also be used in animal models. The results of such in vitro and animal in vivo uses of the compounds and pharmaceutical compositions can, for example, inform their in vivo use in humans, or they can be valuable independent of any human therapeutic or prophylactic use.

### EXAMPLE EMBODIMENTS

1. A compound having a structure

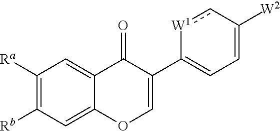

wherein
W¹ is CH, CH₂, N, or NH;
W² is Br, Cl, F, phenyl, CF₃, lower alkyl, C(CH₃)₃, heteroaryl, cycloalkyl, OW^{a}, OCH₂W^{a}, OCH₂W^{b}, or NHSO₂W^{b}, NW^{c}SO₂W^{c};
W^{a} is Br, aryl, CF₃, lower alkyl, cycloalkyl, heterocycloalkyl, CHF₂, C(CH₃)₃, or NHSO₂W^{b};
W^{b} is phenyl, cycloalkyl, heterocycloalkyl, or lower alkyl;
W^{c} is lower alkyl;
R^{a} is H, lower alkyl or OR^{c}, where R^{c} is H or lower alkyl;
R^{b} is phenyl, phenol, OR^{d}, NR^{d}, OR^{d}R^{e}, or NR^{d}R^{e}
R^{d} is lower alkyl, alkylsulfonyl, SO₂CH₃, alkylcarbonyl, CF₂, C(═O)NHR^{c}, CH₂C(═O)R^{f}, CH₂C(═O)R^{f}R^{g}, CH₂R^{h}, CH₂CH₂R^{f}, CH₂CH₂R^{f}R^{g}, CH₂CH₂R^{f}Rⁱ,
R^{e} is hydroxyl, lower alkyl, alkylsulfonyl, or NHR^{c};
R^{f} is heteroaryl or heterocycloalkyl,
R^{g} is alkylcarbonyl, alkylsulfonyl, or lower alkyl,
R^{h} is alkynyl, and
the dashed lines represent the presence or absence of a double bond.
2. A compound of embodiment 1, wherein W1 is N, W2 is lower alkyl, and Rb is ORi, where Ri is alkylcarbonyl.
3. A compound of embodiment 1, wherein W2 is Br, CF3, OCF3, or C(CH3)3 and Rb is ORj, where Rj is sulfonyl.
4. A compound of embodiment 1, wherein W2 is C(CH3)3 and Rb is NCH3Rj, where Rj is sulfonyl.
5. A compound having a structure

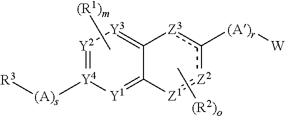

wherein
R1 and R2 are each independently selected from H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH2, OH, CN, NO2, OCF3, CF3, Br, Cl, F, 1-amidino, 2-amidino, alkylcarbonyl, morpholino, piperidyl, N-alkyl piperizinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR4, SOR4, SO₂R4, CO2R4, COR4, CONR4R5, CH2CONR4R5, NR4SO2R5, CSNR4R5, or SOmNR4R5;
R3 is H, R1, alkylsulfonyl, NR4SO2R5, SOmNR4R5, lower alkyl, aryl, alkenyl, alkynyl, haloalkyl, alkylaryl, arylalkyl, alkoxyalkylaryl, alkylamino, arylamino, heteroalkyl, heteroaryl, cyclic heteroalkyl, acyl, arylsulfonyl, or heterocyclicalkylalkyl;
R4 and R5 are each independently selected from H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH2, OH, CN, NO2, OCF3, CF3, Br, Cl, F, 1-amidino, 2-amidino, alkylcarbonyl, morpholino, piperidyl, N-alkyl piperizinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, or isoquinoline;
A and A′ are each independently selected from O, S, or NR′, where R′ is H, lower alkyl or R3, or R′ and R3 or R′ and W can come together to form an unsubstituted or substituted heterocyclic ring or heteroaryl ring;
W is aryl, substituted aryl, heteroaryl, substituted heteroaryl, alkyl, substituted alkyl, cycloalkyl, substituted cycloalkyl, heteroalkyl, substituted heteroalkyl, heterocycloalkyl, substituted heterocycloalkyl, arylalkyl, or heteroaryl alkyl;
Z1, Z2, and Z3 are each independently selected from C, O, NH, S, C═O, S═O or SO2;
Y1, Y2, Y3, and Y4 are each independently selected from C or N, provided that when Y4 is N, then R3-(A)s is not present;
the dashed lines represent the presence or absence of a double bond;
m is 1 or 2;
n is 0, 1, 2 or 3;
o is 0, 1, 2, or 3;
s is 0 or 1; and
r is 0 or 1.
6. A compound of embodiment 5, wherein the compound has a structure

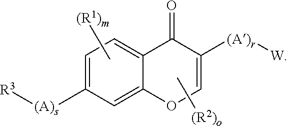

7. A compound of embodiment 5, wherein Y4 is N.
8. A compound of embodiment 5, wherein W has a structure selected from:

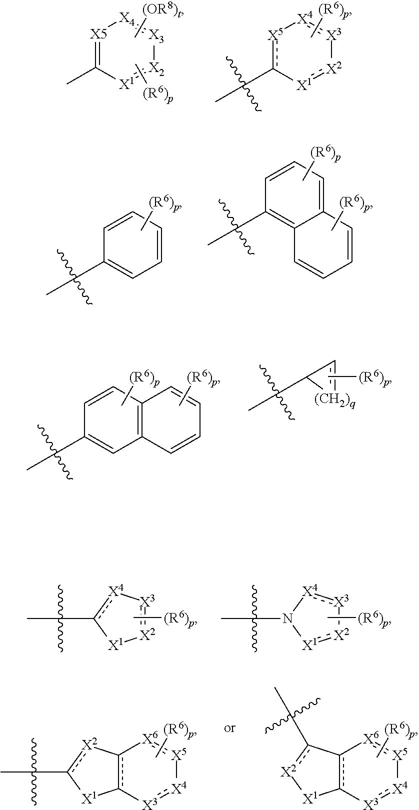

wherein
each of X1, X2, X3, X4, X5, and X6 are independently selected from C, O, NH, NR6, S, C═O, S═O, or SO2;
each R6 is independently selected from H, lower alkyl, haloalkyl, cycloalkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH2, OH, CN, NO2, OCF3, CF3, Br, Cl, F, 1-amidino, 2-amidino, alkylcarbonyl, morpholino, piperidyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, N-alkyl piperazinyl, quinoline, isoquinoline, SR4, SOR4, SO2R4, CO2R4, COR4, CONR4R5, NR4SO2R5, CSNR4R5, or SOmNR4R5, or two adjacent R6 groups can come together to form a fused 5- or 6-membered cycloalkyl ring, heterocycloalkyl ring, methylene dioxo ring, ethylene dioxo ring, aryl ring, or heteroaryl ring;
each R8 is independently selected from H, alkyl, haloalkyl, cycloalkyl, aryl, alkenyl, alkynyl, alkylaryl, arylalkyl, alkoxyalkylaryl, heteroalkyl, heteroaryl, cyclic heteroalkyl, acyl, CF₃, alkylcarbonyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, CO2R4, COR4, CONR4R5, SO2CH3, or two adjacent R8 groups can come together to form a fused 5- or 6-membered cycloalkyl ring, heterocycloalkyl ring, methylene dioxo ring, ethylene dioxo ring, aryl ring or heteroaryl ring; p and t are each independently 0, 1, 2, 3, 4, or 5, provided that p+t≦5; and
q is 1, 2, 3, or 4.
9. A compound of embodiment 8, wherein R6 is H, methyl, ethyl, propyl, isopropyl, butyl, sec-butyl, isobutyl, tert-butyl, Cl, Br, CF3, OCF3, or —NHSO2R7, where R7 is lower alkyl, cycloalkyl, heterocycloalkyl, aryl, or heteroaryl.
10. A compound of embodiment 9 wherein R7 is N-piperidyl, N-morpholino, N-alkyl-N-piperazinyl, or phenyl.
11. A compound of embodiment 8, wherein r is 0 and W is 1-naphthyl, cyclopentyl, 2-thiazolyl, 2-pyrazinyl, 2-benzoxazolyl, or 4-R6-1-phenyl and R6 is tert-butyl, Br, OCF3, or —NHSO2R7, where R7 is N-piperidyl or phenyl; or
r is 1, and W is phenyl.
12. A compound of embodiment 8, wherein r is 0 and W is 4-(OR8)-1-phenyl and (OR8) is trifluoromethoxy, butanyloxy, cyclopropylmethoxy, dimethylpropoxy, trifluoroethoxy, difluoromethoxy, oxanylmethoxy, oxanylmethoxy, or dimethylbutoxy.
13. A compound of embodiment 5, wherein s is 1, A is O or NR′ where R′ is H or lower alkyl, and R3 is H, 3-propynyl, SO2CH3, CF2H, CF3, CONHCH3, or CH2CONR4R5; where R4 and R5 come together to form a morpholino ring, an N-acetyl piperazinyl ring, an N-methanesulfonyl piperazinyl ring, or an N-methyl piperazinyl ring; or
s is 0 and R3 is SO₂CH₃, COR4, CONR4R5, N-imidazolinyl, or N-maleimido.
14. A compound of embodiment 5, wherein the compound has a structure selected from:

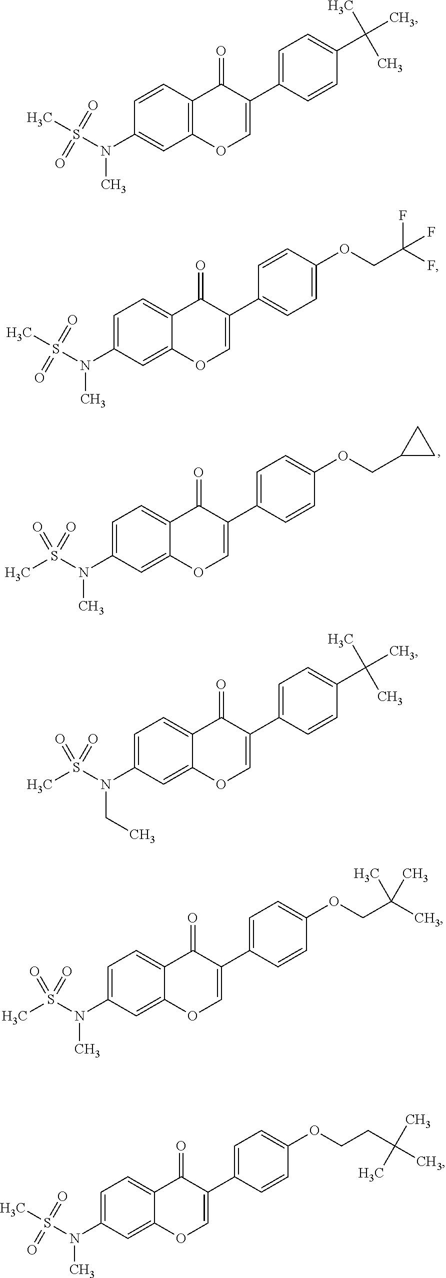 [SMILES: CN(c1ccc2c(=O)c(-c3ccc(C(C)(C)C)cc3)coc2c1)S(C)(=O)=O ; CN(c1ccc2c(=O)c(-c3ccc(OCC(F)(F)F)cc3)coc2c1)S(C)(=O)=O ; CN(c1ccc2c(=O)c(-c3ccc(OCC4CC4)cc3)coc2c1)S(C)(=O)=O ; CCN(c1ccc2c(=O)c(-c3ccc(C(C)(C)C)cc3)coc2c1)S(C)(=O)=O ; CN(c1ccc2c(=O)c(-c3ccc(OCC(C)(C)C)cc3)coc2c1)S(C)(=O)=O ; CN(c1ccc2c(=O)c(-c3ccc(OCCC(C)(C)C)cc3)coc2c1)S(C)(=O)=O]

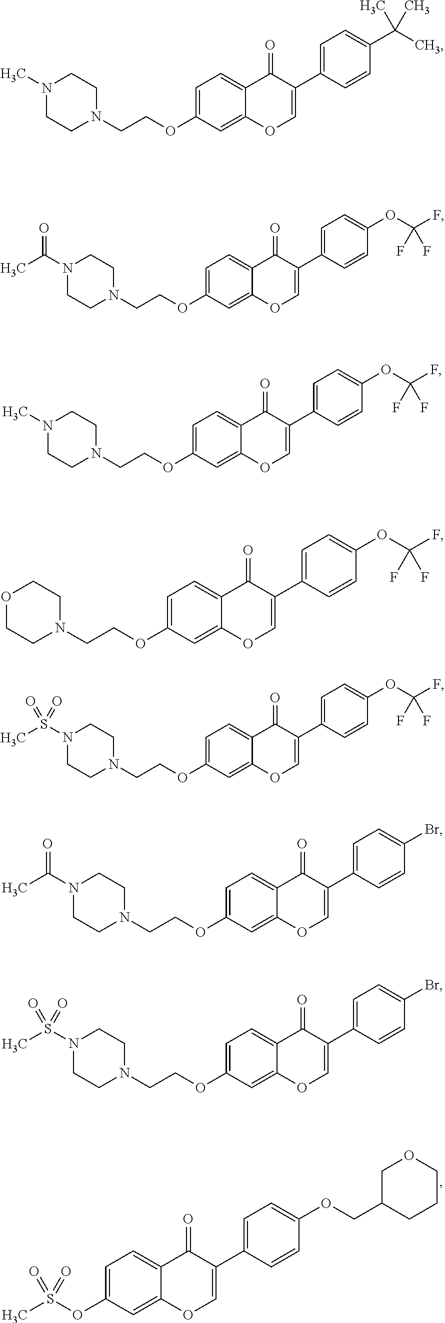

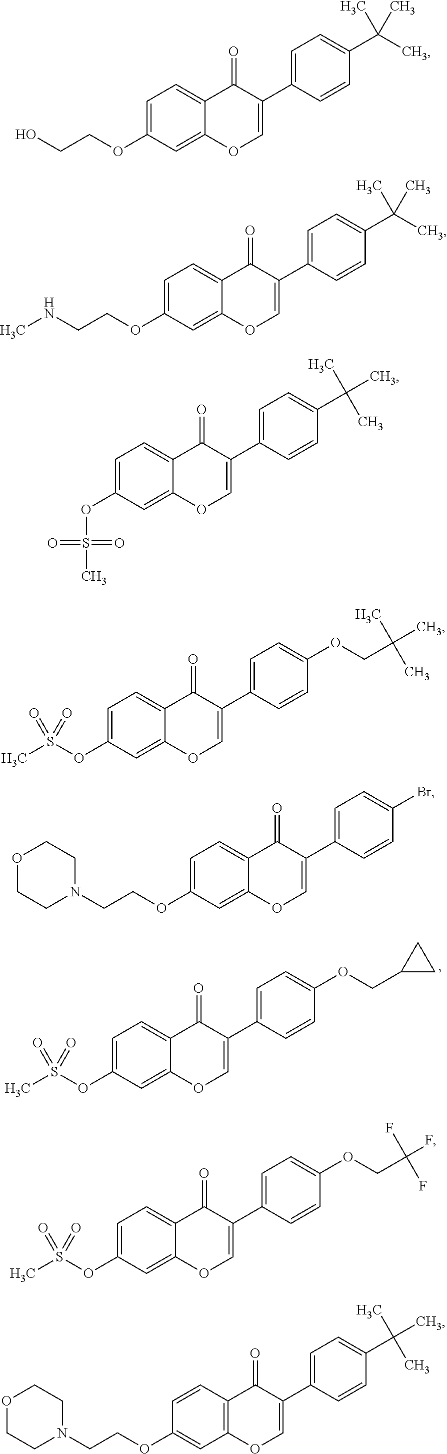

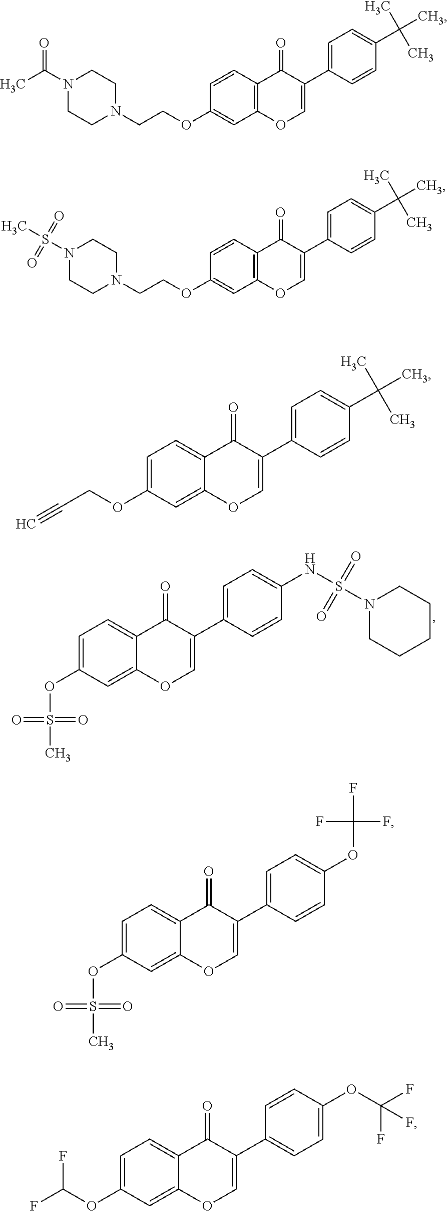 [SMILES: CC(=O)N1CCN(CCOc2ccc3c(=O)c(-c4ccc(C(C)(C)C)cc4)coc3c2)CC1 ; CC(C)(C)c1ccc(-c2coc3cc(OCCN4CCN(S(C)(=O)=O)CC4)ccc3c2=O)cc1 ; C#CCOc1ccc2c(=O)c(-c3ccc(C(C)(C)C)cc3)coc2c1 ; CS(=O)(=O)Oc1ccc2c(=O)c(-c3ccc(NS(=O)(=O)N4CCCCC4)cc3)coc2c1 ; CS(=O)(=O)Oc1ccc2c(=O)c(-c3ccc(OC(F)(F)F)cc3)coc2c1 ; O=c1c(-c2ccc(OC(F)(F)F)cc2)coc2cc(OC(F)F)ccc12]

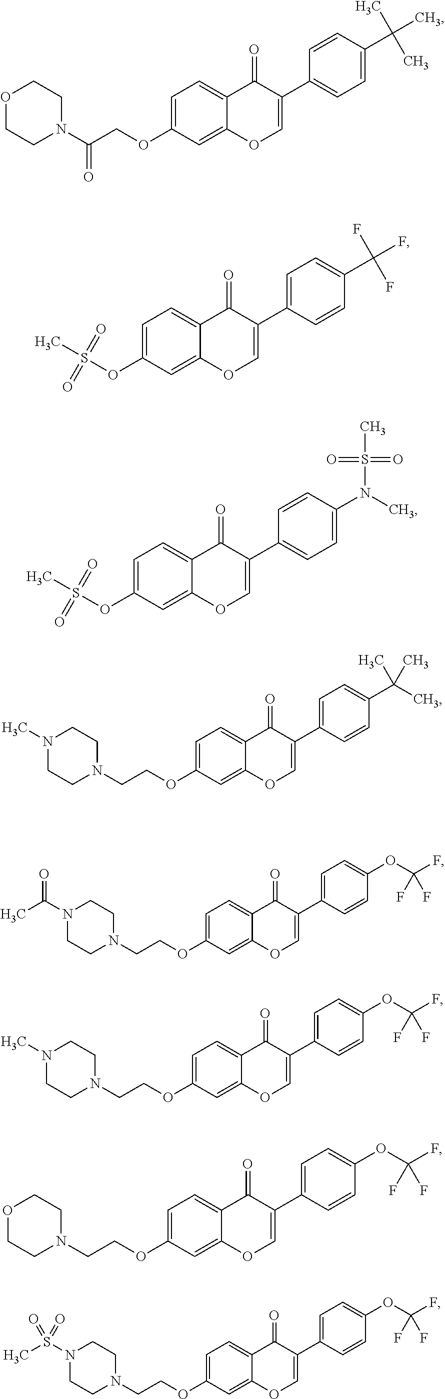

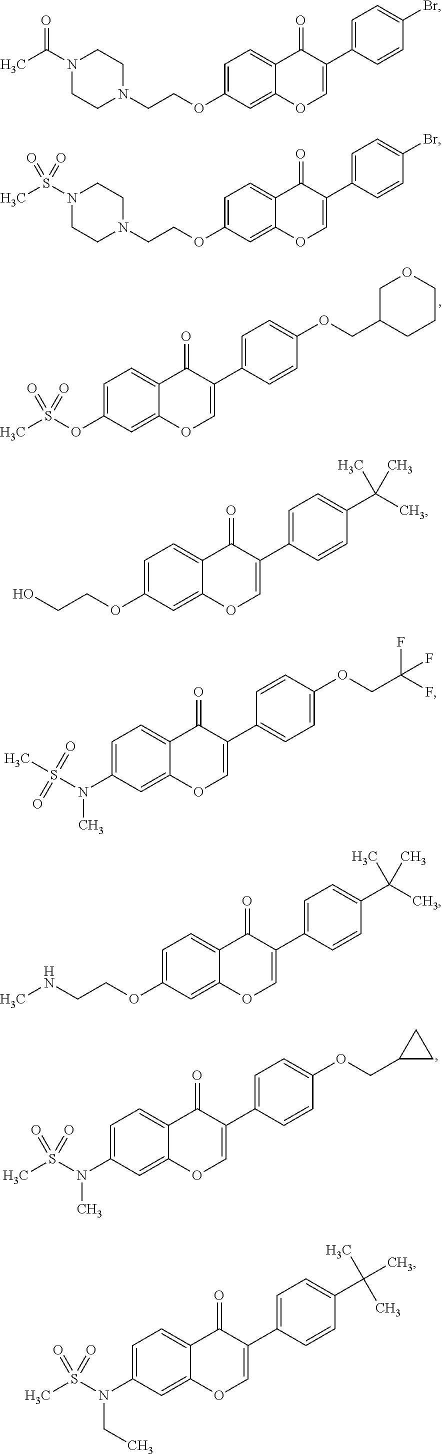

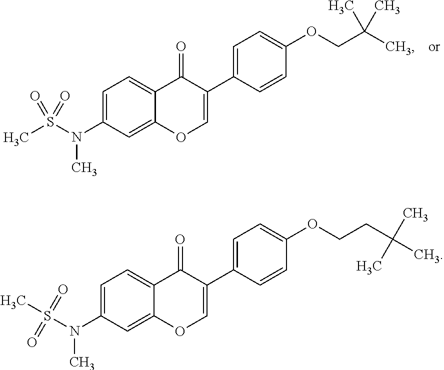 [SMILES: CN(c1ccc2c(=O)c(-c3ccc(OCC(C)(C)C)cc3)coc2c1)S(C)(=O)=O ; CN(c1ccc2c(=O)c(-c3ccc(OCCC(C)(C)C)cc3)coc2c1)S(C)(=O)=O]

15. A pharmaceutical composition comprising a compound of any one of embodiments 1 to 14.
16. A pharmaceutical composition of embodiment 15, for use in therapy.
17. A pharmaceutical composition for use according to embodiment 16, wherein the compound has a structure as shown in embodiment 14.
18. A pharmaceutical composition for use according to embodiments 16 or 17, wherein said pharmaceutical composition is administered as an adjuvant for a prophylactic or therapeutic vaccine.
19. A pharmaceutical composition for use according to embodiment 18, wherein said use comprises vaccinating a subject by additionally administering a vaccine against Alfuy virus, Banzi virus, bovine diarrhea virus, Chikungunya virus, DNV, EMCV, HBV, HCV, hCMV, HIV, Ilheus virus, influenza virus (including avian and swine isolates), Japanese encephalitis virus, Kokobera virus, Kunjin virus, Kyasanur forest disease virus, louping-ill virus, measles virus, MERS, metapneumovirus, any of the Mosaic Viruses, Murray Valley virus, parainfluenza virus, poliovirus, Powassan virus, RSV, Rocio virus, SARS, St. Louis encephalitis virus, tick-borne encephalitis virus, WNV, and yellow fever virus.
20. A pharmaceutical composition of embodiment 15, for use in treating a viral infection in a subject.
21. A pharmaceutical composition for use according to embodiment 20, wherein the viral infection is caused by a virus from one or more of the following families: Arenaviridae, Arterivirus, Astroviridae, Birnaviridae, Bromoviridae, Bunyaviridae, Caliciviridae, Closteroviridae, Comoviridae, Coronaviridae, Cystoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Paramyxoviridae, Picobirnaviridae, Picobirnavirus, Picornaviridae, Potyviridae, Reoviridae, Retroviridae, Roniviridae, Sequiviridae, Tenuivirus, Togaviridae, Tombusviridae, Totiviridae, and Tymoviridae.
22. A pharmaceutical composition for use according to embodiments 17 or 18, wherein the viral infection is Alfuy virus, Banzi virus, bovine diarrhea virus, Chikungunya virus, Dengue virus (DNV), encephalomycarditis virus (EMCV) Hepatitis B virus (HBV), Hepatitis C virus (HCV), human cytomegalovirus (hCMV), human immunodeficiency virus (HIV), Ilheus virus, influenza virus (including avian and swine isolates), Japanese encephalitis virus, Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), and yellow fever virus.
23. A pharmaceutical composition for use according to any one of embodiments 20 to 22, wherein the viral infection is caused by HCV.
24. A pharmaceutical composition for use according to any one of embodiments 20 to 22, wherein the viral infection is caused by EMCV.
25. A pharmaceutical composition for use according to any one of embodiments 20 to 22, wherein the viral infection is caused by RSV.
26. A pharmaceutical composition for use according to any one of embodiments 20 to 22, wherein the viral infection is caused by influenza virus.
27. A pharmaceutical composition for use according to any one of embodiments 20 to 22, wherein the viral infection is caused by DNV.
28. A pharmaceutical composition for use according to any one of embodiments 20 to 22, wherein the viral infection is caused by hCMV.
29. A pharmaceutical composition for use according to embodiment 20, wherein said pharmaceutical composition is administered as an adjuvant for a prophylactic or therapeutic vaccine.
30. A pharmaceutical composition for use according to embodiment 29, wherein said use comprises vaccinating a subject by additionally administering a vaccine against Alfuy virus, Banzi virus, bovine diarrhea virus, Chikungunya virus, DNV, HBV, HCV, hCMV, HIV, Ilheus virus, influenza virus (including avian and swine isolates), Japanese encephalitis virus, Kokobera virus, Kunjin virus, Kyasanur forest disease virus, louping-ill virus, measles virus, MERS, metapneumovirus, any of the Mosaic Viruses, Murray Valley virus, parainfluenza virus, poliovirus, Powassan virus, RSV, Rocio virus, SARS, St. Louis encephalitis virus, tick-borne encephalitis virus, WNV, and yellow fever virus.
31. A pharmaceutical composition for use according to any one of embodiments 20 to 30, wherein the compound has a structure as shown in embodiment 14.
32. A method of treating a viral infection in a subject comprising administering to the subject a therapeutically effective amount of a pharmaceutical composition of embodiment 15, thereby treating the viral infection in the subject.
33. A method of embodiment 32, wherein the viral infection is caused by a virus from one or more of the following families: Arenaviridae, Arterivirus, Astroviridae, Birnaviridae, Bromoviridae, Bunyaviridae, Caliciviridae, Closteroviridae, Comoviridae, Coronaviridae, Cystoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Paramyxoviridae, Picobirnaviridae, Picobirnavirus, Picornaviridae, Potyviridae, Reoviridae, Retroviridae, Roniviridae, Sequiviridae, Tenuivirus, Togaviridae, Tombusviridae, Totiviridae, and Tymoviridae.
34. A method of embodiments 32 or 33, wherein the viral infection is caused by one or more of influenza virus, Alfuy virus, Banzi virus, bovine diarrhea virus, Chikungunya virus, DNV, EMCV, HBV, HCV, hCMV, HIV, Ilheus virus, influenza virus (including avian and swine isolates), Japanese encephalitis virus, Kokobera virus, Kunjin virus, Kyasanur forest disease virus, louping-ill virus, measles virus, MERS, metapneumovirus, any of the Mosaic Viruses, Murray Valley virus, parainfluenza virus, poliovirus, Powassan virus, RSV, Rocio virus, SARS, St. Louis encephalitis virus, tick-borne encephalitis virus, WNV, and yellow fever virus.
35. A method of any one of embodiments 32 to 34, wherein the viral infection is caused by HCV.
36. A method of any one of embodiments 32 to 34, wherein the viral infection is caused by EMCV.
37. A method of any one of embodiments 32 to 34, wherein the viral infection is caused by RSV.
38. A method of any one of embodiments 32 to 34, wherein the viral infection is caused by influenza virus.
39. A method of any one of embodiments 32 to 34, wherein the viral infection is caused by DNV.
40. A method of any one of embodiments 32 to 34, wherein the viral infection is caused by hCMV.
41. A method of any one of embodiments 32 to 34, wherein the pharmaceutical composition is administered as an adjuvant for a prophylactic or therapeutic vaccine.
42. A method of embodiment 41 wherein the method comprises vaccinating a subject by additionally administering a vaccine against Alfuy virus, Banzi virus, bovine diarrhea virus, Chikungunya virus, DNV, HBV, HCV, hCMV, HIV, Ilheus virus, influenza virus (including avian and swine isolates), Japanese encephalitis virus, Kokobera virus, Kunjin virus, Kyasanur forest disease virus, louping-ill virus, measles virus, MERS, metapneumovirus, any of the Mosaic Viruses, Murray Valley virus, parainfluenza virus, poliovirus, Powassan virus, RSV, Rocio virus, SARS, St. Louis encephalitis virus, tick-borne encephalitis virus, WNV, and yellow fever virus.
43. A method of any one of embodiments 32 to 42, wherein the compound has a structure as shown in embodiment 14.
44. A compound of any one of embodiments 1 to 14 for use in modulating an innate immune response in a eukaryotic cell, the use comprising administering the compound to the eukaryotic cell.
45. A compound for use according to embodiment 44, wherein the cell is in vivo.
46. A compound for use according to embodiment 44, wherein the cell is in vitro.
47. A compound for use according to embodiments 44 or 46, wherein the cell is a Huh7 cell.
48. A compound for use according to embodiments 44 or 46, wherein the cell is a HeLa cell.
49. A compound for use according to embodiments 44 or 46, wherein the cell is a 293 cell.
50. A method of modulating the innate immune response in a eukaryotic cell, comprising administering to the cell a compound of any one of embodiments 1 to 14.
51. A method of embodiment 50, wherein the cell is in vivo.
52. A method of embodiment 50, wherein the cell is in vitro.
53. A method of embodiments 50 or 52, wherein the cell is a Huh7 cell.
54. A method of embodiments 50 or 52, wherein the cell is a HeLa cell.
55. A method of embodiments 50 or 52, wherein the cell is a 293 cell.

The Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure. For example, the Examples below provide in vitro methods for testing the compounds of the disclosure. Other in vitro and/or in vivo virus infection models include flaviviruses such as DNV, bovine diarrheal virus, WNV, and GBV-C virus, other RNA viruses such as RSV, SARS, and the HCV replicon systems. Furthermore, any appropriate cultured cell competent for viral replication can be utilized in the antiviral assays.

### EXAMPLES

### Example 1

### Synthesis of Compounds of the Disclosure

General synthetic scheme. The compounds of the disclosure may be prepared by the methods described below, together with synthetic methods familiar to those of ordinary skill in the art. The starting materials used herein are commercially available or can be prepared by routine methods known in the art (such as those methods disclosed in standard reference books such as the COMPENDIUM OF ORGANIC SYNTHETIC METHODS, Vol. I-VI (published by Wiley-Interscience)). Preferred methods include those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; T. W. Greene and P. G. M. Wuts, Protective Groups in Organic Chemistry, John Wiley & Sons, 1991, and T. W. Greene and P. G. M. Wuts, Protective Groups in Organic Chemistry, John Wiley & Sons, 1999.

Compounds of the disclosure, or their pharmaceutically acceptable salts, can be prepared according to the reaction schemes discussed below. These methods can be modified or adapted in ways known to chemists of ordinary skill in order to achieve synthesis of additional compounds within the scope of the present disclosure. Such modification was performed to synthesize an example compound of the disclosure as described in Examples 2-4. Unless otherwise indicated, the substituents in the schemes are defined as above. Isolation and purification of the products is accomplished by standard procedures, which are known to a chemist of ordinary skill.

It will be understood by one skilled in the art that the various symbols, superscripts, and subscripts used in the schemes, methods, and examples are used for convenience of representation and/or to reflect the order in which they are introduced in the schemes, and are not intended to necessarily correspond to the symbols, superscripts, or subscripts in the appended claims. The schemes are representative of methods useful in synthesizing the compounds of the present disclosure. They are not to constrain the scope of the disclosure in any way.

Isoflavones may be prepared by a wide variety of methods reviewed in publications including T.A. Geissman The Chemistry of Flavonoid Compounds, MacMillan, New York, 1962; P.M. Dewick Isoflavonoids. In The Flavonoids: Advances in Research, J. B. Harborne and T. J. Mabry, Eds. Chapman & Hall, New York, 1982; E. Wong The Isoflavonoids. In The Flavonoids, J. B. Harborne, T. J. Mabry, and Helga Mabry, Eds., Academic Press, New York San Francisco, 1975; Paul M. Dewick Isoflavonoids. In The Flavonoids: Advances in research since 1986, J. B. Harborne, Ed., Chapman & Hall, London, 1993; Lévai, A. (2004), Synthesis of isoflavones. J. Heterocyclic Chem., 41: 449-460; John A. Joule, Keith Mills, Heterocyclic Chemistry, Wiley & Sons, 5th Ed, 2009; and Mamoalosi A. Selepe and Fanie R. Van Heerden, Application of the Suzuki-Miyaura reaction in the synthesis of flavonoids, Molecules (2013), 18, 4739-4765. Scheme 1 to Scheme 7 shown below summarize some of the common methods used to construct isoflavones. 1-(2-Hydroxyphenyl)-2-phenylethanone intermediates of the disclosure may be prepared by acylation of a suitably substituted phenol by a variety of methods including those shown in Scheme 8.

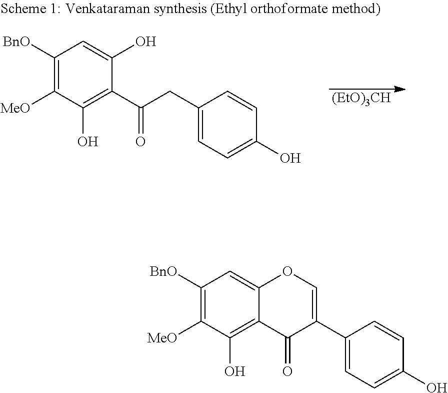 [SMILES: "(EtO)3CH" ; COc1c(OCc2ccccc2)cc(O)c(C(=O)Cc2ccc(O)cc2)c1O ; COc1c(OCc2ccccc2)cc2occ(-c3ccc(O)cc3)c(=O)c2c1O]

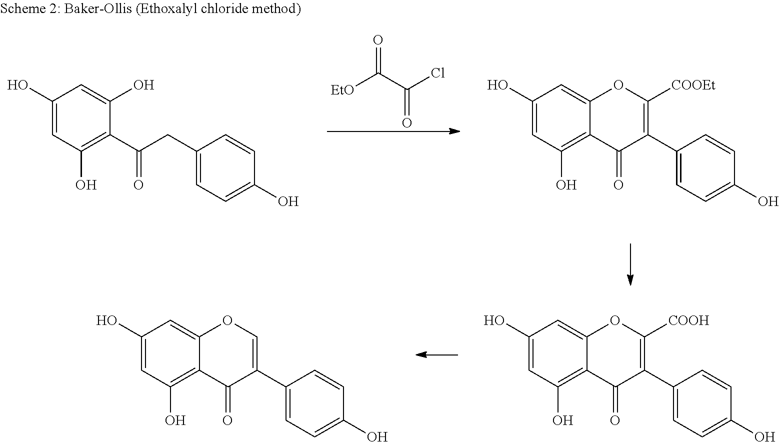 [SMILES: O=C(Cc1ccc(O)cc1)c1c(O)cc(O)cc1O ; CCOC(=O)C(=O)Cl ; CCOC(=O)c1oc2cc(O)cc(O)c2c(=O)c1-c1ccc(O)cc1 ; O=c1c(-c2ccc(O)cc2)coc2cc(O)cc(O)c12 ; O=C(O)c1oc2cc(O)cc(O)c2c(=O)c1-c1ccc(O)cc1]

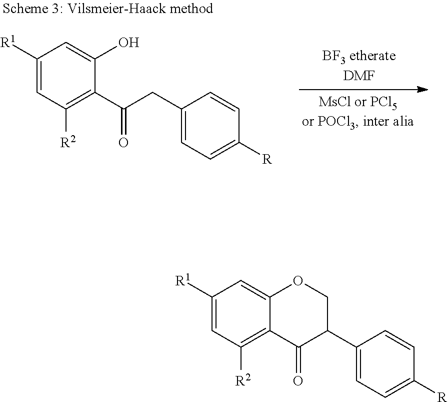

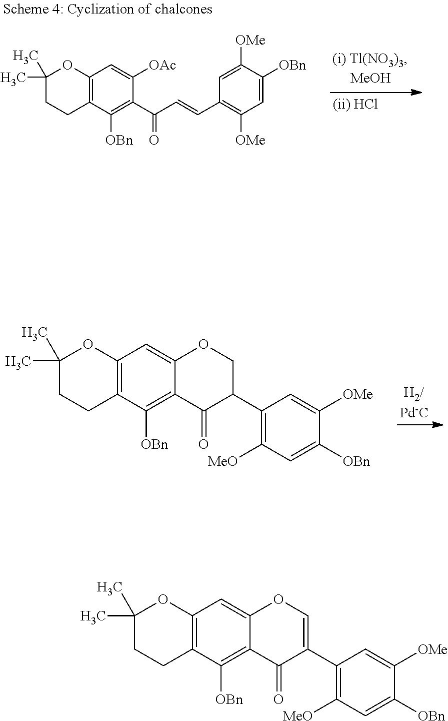 [SMILES: COc1cc(OCc2ccccc2)c(OC)cc1/C=C/C(=O)c1c(OC(C)=O)cc2c(c1OCc1ccccc1)CCC(C)(C)O2 ; COc1cc(C2COc3cc4c(c(OCc5ccccc5)c3C2=O)CCC(C)(C)O4)c(OC)cc1OCc1ccccc1 ; COc1cc(-c2coc3cc4c(c(OCc5ccccc5)c3c2=O)CCC(C)(C)O4)c(OC)cc1OCc1ccccc1]

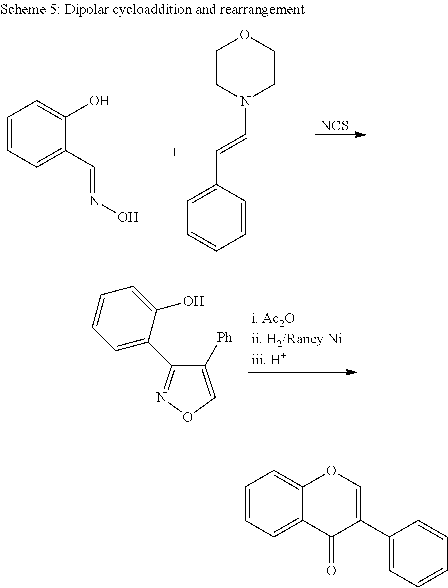 [SMILES: O/N=C/c1ccccc1O ; C(=C\N1CCOCC1)/c1ccccc1 ; "NCS" ; Oc1ccccc1-c1nocc1-c1ccccc1 ; O=c1c(-c2ccccc2)coc2ccccc12]

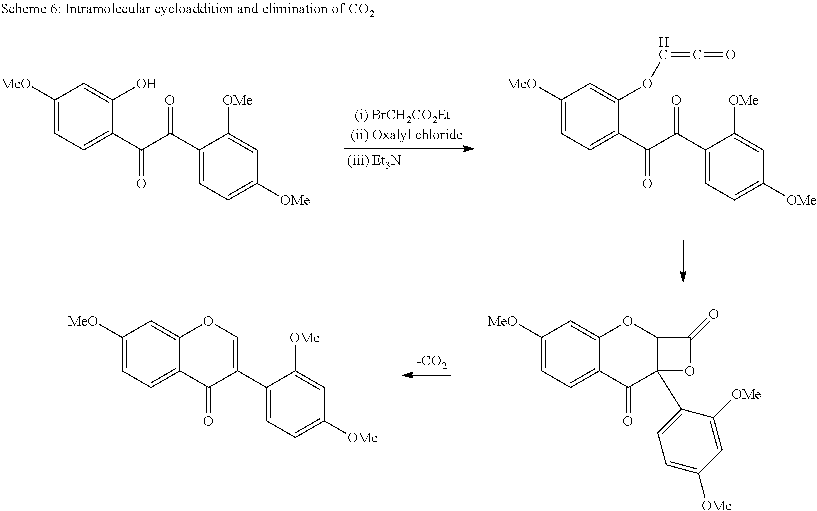 [SMILES: COc1ccc(C(=O)C(=O)c2ccc(OC)cc2OC)c(O)c1 ; COc1ccc(C(=O)C(=O)c2ccc(OC)cc2OC=C=O)c(OC)c1 ; COc1ccc(-c2coc3cc(OC)ccc3c2=O)c(OC)c1 ; COc1ccc2c(c1)OC1C(=O)OC1(c1ccc(OC)cc1OC)C2=O]

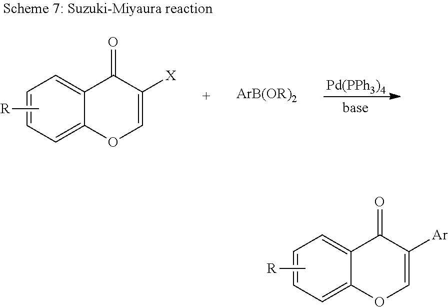

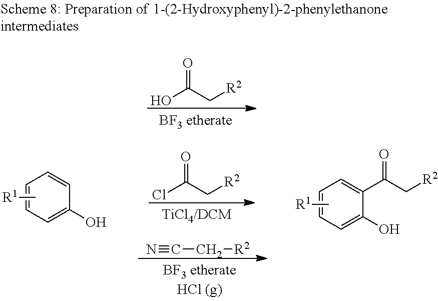

### Example 2

### Synthesis of 3-(4-tert-butylphenyl)-4-oxo-4H-chromen-7-yl methanesulfonate

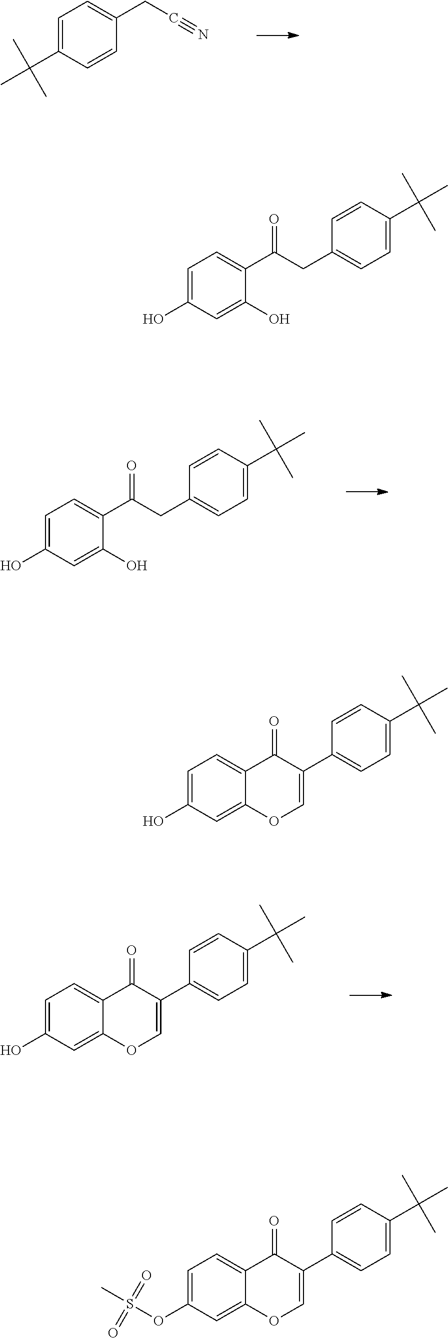

Step 1: Synthesis of the intermediate 2-(4-tert-butylphenyl)-1-(2,4-dihydroxyphenyl)ethanone. (4-tert-butylphenyl)acetonitrile (10 g, 0.058 mol) and resorcinol (7.3 g, 0.066 mol) were added to 40 mL BF3.Et2O and a stream of dry HCl gas was passed through the mixture overnight. The solution was then poured into 300 mL cold water and stirred 6 hours. The mixture was extracted with ethyl acetate and evaporation of the solvent afforded an oil which was purified by chromatography to afford 0.68 g of 3-(4-tert-butylphenyl)-7-hydroxy-4H-chromen-4-one (20%) after chromatography.

Step 2: Synthesis of 3-(4-tert-butylphenyl)-7-hydroxy-4H-chromen-4-one. The intermediate of Step 1 (0.65 g, 2.3 mmol) was mixed with 1:1 triethyl orthoformate and dry pyridine, and piperidine and was held at 120-130° C. for 4 hours. The mixture was allowed to cool and added to water. The precipitated solid was filtered off and recrystallized from chloroform to afford 0.324 g of product (45%).

Step 3: Synthesis of 3-(4-tert-butylphenyl)-4-oxo-4H-chromen-7-yl methanesulfonate. Methanesulfonyl chloride (0.079 mL, 1 mmol) was added dropwise to a solution of the product of Step 2 (0.15 g, 0.5 mmol) and 0.2 mL triethylamine in 10 mL. The mixture was stirred at room temperature for 16 hours. The solvent was evaporated to dryness and the residue was triturated with methanol to afford the methanesulfonate ester, (0.16 g, 84%)

### Example 3

### Synthesis of N-[3-(4-tert-butylphenyl)-4-oxo-4H-chromen-7-yl]-N-methylmethanesulfonamide

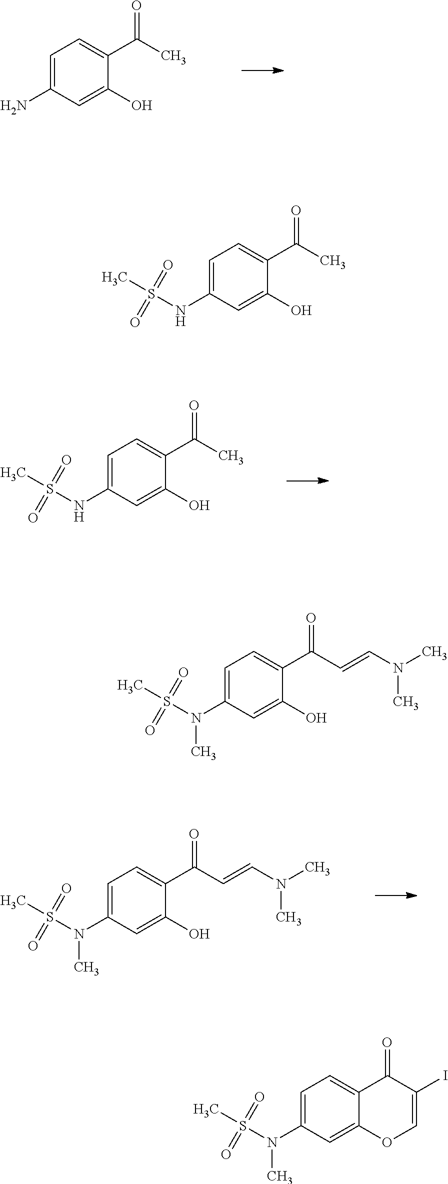

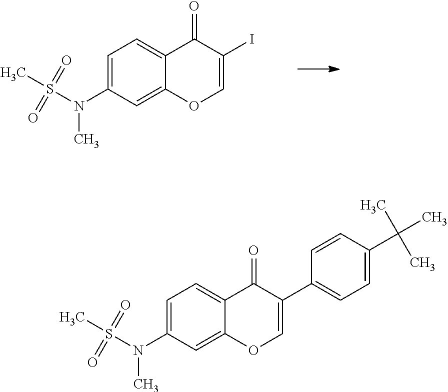 [SMILES: CN(c1ccc2c(=O)c(I)coc2c1)S(C)(=O)=O ; CN(c1ccc2c(=O)c(-c3ccc(C(C)(C)C)cc3)coc2c1)S(C)(=O)=O]

Step 1: Synthesis of N-(4-acetyl-3-hydroxyphenyl)methanesulfonamide. Pyridine (1.6 mL, 20 mmol) was added at 0° C. to a mixture of commercially available 4′-amino-2′-hydroxyacetophenone (2 g, 13 mmol) and methanesulfonyl chloride (1.6 mL, 16 mmol) in 40 mL of anhydrous dichloromethane. The resulting mixture was stirred at 0° C. to room temperature overnight before being diluted with dichloromethane and washed with 1M aqueous hydrogen chloride. Insoluble material appeared at the interface between the two layers. The aqueous layer was back extracted twice with dichloromethane. The combined organic layers were dried over sodium sulfate, filtered, and evaporated to give 0.11 g of sulfonamide. The insoluble material at the interface between the two extraction layers was filtered and rinsed with diethyl ether to give 1.3 g of sulfonamide (89% yield).

Step 2: Synthesis of N-{4-[(2E)-3-(dimethylamino)prop-2-enoyl]-3-hydroxyphenyl}methanesulfonamide. 2 mL of dimethylformamide dimethyl acetal were added to a solution of the product of Step 1 (0.5 g, 2 mmol) in 1 mL of dimethylformamide. The resulting mixture was stirred at 95° C. for one hour before being cooled to room temperature. Water was added drop-wise until a yellow precipitate formed. The precipitate was filtered, rinsed with water, and dried under vacuum to give 0.17 g of product (26% yield).

Step 3: Synthesis of N-(3-iodo-4-oxo-4H-chromen-7-yl)-N-methylmethanesulfonamide. Iodine (0.21 g, 0.83 mmol) was added at 0° C. to a solution of the product of Step 2 (0.17 g, 0.57 mmol) in 5 mL of chloroform. The resulting mixture was stirred at 0° C. to room temperature overnight before being quenched by addition of saturated aqueous sodium thiosulfate. The aqueous layer was back extracted twice with dichloromethane. The combined organic layers were dried over sodium sulfate, filtered, and evaporated. The residue was taken into ethyl acetate and the insoluble material was filtered, rinsed with ethyl acetate, and dried under vacuum to give 0.14 g of the iodochromene (65% yield).

Step 4: Synthesis of N-[3-(4-tert-butylphenyl)-4-oxo-4H-chromen-7-yl]-N-methylmethanesulfonamide. A mixture of the product of Step 3 ((0.07 g, 0.19 mmol), 4-tert-butylphenylboronic acid (0.043 g, 0.24 mmol), palladium 10% on charcoal (0.01 g), and sodium carbonate (0.059 g, 0.56 mmol) in 1.5 mL of a 1/1 mixture of 1,2-dimethoxyethane and water was stirred at 45-50° C. for two hours before being partitioned between dichloromethane and water. The aqueous layer was back extracted twice with dichloromethane. The combined organic layers were dried over sodium sulfate, filtered, and evaporated. The residue was taken into methanol and the insoluble material was filtered, rinsed with methanol, and dried under vacuum to give 0.052 g of the isoflavone (73% yield).

### Example 4

### Synthesis of 4-oxo-3-[4-(2,2,2-trifluoroethoxy)phenyl]-4H-chromen-7-yl methanesulfonate

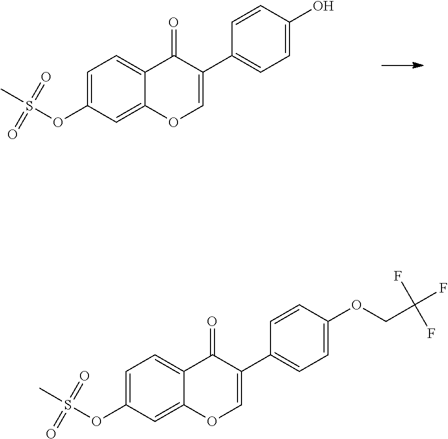 [SMILES: CS(=O)(=O)Oc1ccc2c(=O)c(-c3ccc(O)cc3)coc2c1 ; CS(=O)(=O)Oc1ccc2c(=O)c(-c3ccc(OCC(F)(F)F)cc3)coc2c1]

The molecule 3-(4-hydroxyphenyl)-4-oxo-4H-chromen-7-yl methanesulfonate was prepared by the general methods described herein; this molecule (1.0 g, 3.0 mmol) was then dissolved in 10 mL dry dimethylformamide (DMF) and treated with a slight excess of sodium hydride in mineral oil. After the evolution of hydrogen had ceased 2,2,2-trifluoroethyl methanesulfonate (1.0 g, 5.6 mmol) was added dropwise and the mixture was left at room temperature overnight. Liquid chromatography-mass spectrometry (LCMS) analysis showed a mixture of 30% desired monoalkylated product and other products included dialkylated material resulting from loss of the methanesulfonate ester. The desired product was isolated by silica gel chromatography.

### Example 5

### In Vitro Antiviral Activity of KIN100 and KIN101

The library hit compounds KIN100 and KIN101 were tested for antiviral activity in vitro. In an HCV focus-forming assay, Huh7 cells were seeded in 96-well plates at a density of 2-5×10³ cells/well. Cells were grown for 16 hours and compounds that were diluted to 5, 10, 20, or 50 uM in media containing 0.5% dimethyl sulfoxide (DMSO) were added to each well. Cells were incubated for 18-24 hours and then infected with 750 pfu HCV2a strain. Diluted virus was added directly to the well and compound was not removed. Infected cells were grown for 24-72 hours post compound treatment and then fixed. Cells were fixed with 4% paraformaldehyde and stained for HCV protein. Primary serum against HCV was used at a 1:3000 dilution. Secondary goat anti-human antibody conjugated to Alexa Fluor 488 dye (Invitrogen) and Hoescht Dye (nuclear staining) were used at a 1:3000 dilution to detect HCV protein and cell nuclei. Following secondary antibody incubation, the monolayers were washed and left in 100 μL PBS for imaging and quantitation using fluorescence microscopy.

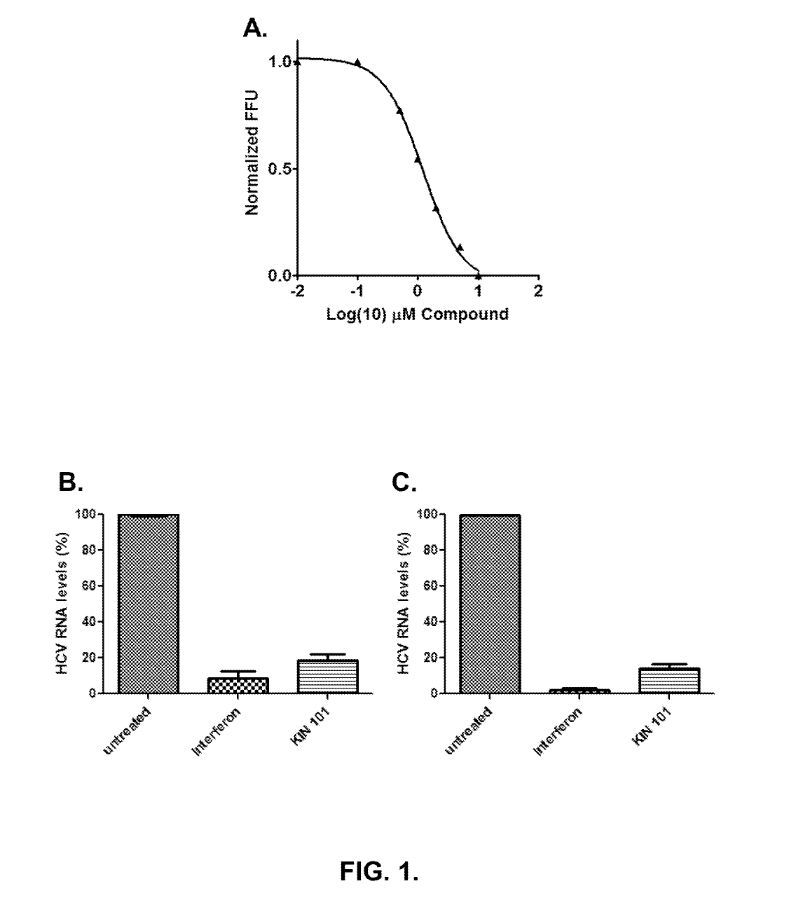

FIGS. 1A-1C show the antiviral activity of KIN100 and KIN101 against HCV. FIG. 1A is a graph of an HCV focus-forming assay performed in Huh7 cells pre-treated with KIN100 for 24 hours and infected with HCV2a at a multiplicity of infection (MOI) of 0.5 for 48 hours. HCV proteins were detected by immunofluorescent staining with viral-specific serum and foci were normalized to negative control cells that were not compound treated (equal to 1). FIG. 1B shows quantitation of HCV viral RNA by RT-qPCR performed in Huh7 cells pre-treated with KIN101 for 18 hours and infected with HCV2a at a MOI of 1.0 for 72 hours. Viral RNA was isolated and quantitated in the supernatant of infected cultures. FIG. 1C shows a similar quantitation of HCV viral RNA by RT-qPCR performed in Huh7 cells infected with HCV2a at a MOI of 1.0 for 4 hours and then treated with KIN101.

In an encephalomyocarditis virus (EMCV) in vitro antiviral assay, Huh7 cells were grown under normal growth conditions and treated with the indicated amount of KIN101 in media containing 0.5% DMSO. The cells were grown in the presence of compound for 5 hours and then infected with 250 pfu Murine EMCV obtained from ATCC #VR-129B. Infected cells were grown for an additional 18 hours and then cell viability was measured using an MTS assay. Negative control cells were treated with buffer alone containing 0.5% DMSO. Interferon treatment was used as a positive control for virus inhibition and was added similar to compound treatments at a final concentration of 10 IU/mL Interferon-α: Intron A, from Schering-Plough. Cell viability was measured using an MTS assay, CellTiter 96® AQueous One Solution Cell Proliferation Assay (MTS), from Promega #G3580. KIN101 was protective of cell viability following infection with EMCV. Assay results are shown below.

TABLE 3
Cell viability following EMCV infection

| Addition (compound or control) | Cell viability post-infection |
|---|---|
| Negative controls | ~0.7−0.75 |
| 5 units interferon | ~1.7 |
| 10 units interferon | ~2.0 |
| 20 units interferon | ~2.25 |
| 5 units KIN101 | ~0.7 |
| 10 units KIN101 | ~1.2 |
| 20 units KIN101 | ~1.45 |

Antiviral activity of KIN101 against RSV was measured by immunofluorescent based focus-forming assay. Cultured human HeLa cells were seeded in 6-well tissue-culture plates at a density of 4×10⁵ cells per well and grown for 24 hours. Cells were infected with RSV A2 Long strain (ATCC VR-26) at a MOI of 0.1 for 2 hours and then removed. Compound dilutions were prepared in 0.5% DMSO and used to treat cells at final concentrations of compound ranging from 0.001 to 10 μM per well. Vehicle control wells contained 0.5% DMSO and were used to compare to compound-treated cells. RSV infections after compound treatment were allowed to proceed for 48 hours. Virus supernatants were then harvested and used to infect new monolayers of HeLa cells seeded in 96-well tissue-culture plates at a density of 8×10³ cells per well. The newly infected cells were incubated overnight (18-24 hours) and used to measure the level of infectious virus in the original supernatants by immunofluorescent staining of viral protein. The cells were fixed with ice-cold 1:1 methanol and acetone solution and stained for RSV F protein. Primary mouse anti-RSV monoclonal antibody (EMD Millipore) was used at a 1:2000 dilution. Secondary goat anti-mouse antibody conjugated to Alexa Fluor 488 dye (Invitrogen) and Hoescht Dye (nuclear staining) were used at a 1:3000 dilution to detect RSV protein and cell nuclei. Following secondary antibody incubation, the monolayers were washed and left in 100 μL PBS for imaging and quantitation using a Cellomics ArrayScan HCS instrument.

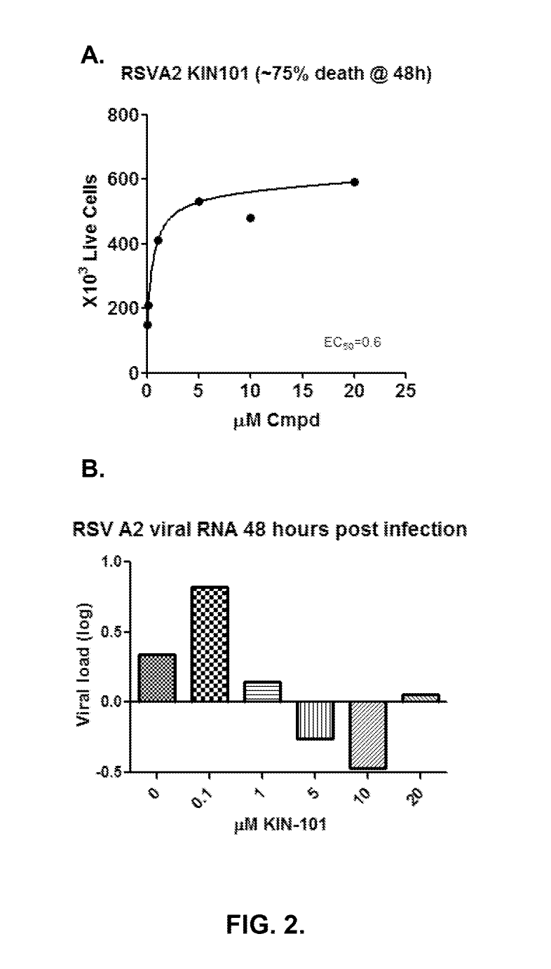

FIG. 2A shows cell viability following infection with RSV A2 and treatment with KIN101. FIG. 2B shows KIN101 treatment decreased RSV viral RNA 48 hours post infection.

### Example 6

### In Vitro Antiviral Activity of KIN269 and Other Selected Compounds

Antiviral activity against influenza virus in vitro was measured for KIN269 and other selected compounds. Cultured human 293 cells were seeded in 6-well tissue-culture plates at a density of 3×10⁵ cells per well for the flu focus-forming assay and grown for 24 hours. Cells were infected with influenza virus A/Udorn/72 H3N2 strain at a MOI of 0.1 for 2 hours and then removed. Compound dilutions were prepared in 0.5% DMSO and used to treat cells at final concentrations of compound ranging from 0.001 to 10 μM per well. Vehicle control wells contained 0.5% DMSO and were used to compare to compound-treated cells. Replication was then allowed to proceed for 24 hours. Virus supernatants were then harvested and used to infect new monolayers of permissive MDCK cells that were seeded 24 hours prior in 96-well tissue-culture plates at a density of 1.5×10⁴ cells per well. The newly infected cells were incubated overnight (18-24 hours) and used to measure the level of infectious virus in the original supernatants by immunofluorescent staining of viral protein. The cells were fixed with ice-cold 1:1 methanol and acetone solution and stained for influenza nucleoprotein (NP). Primary mouse anti-NP monoclonal antibody (Chemicon) was used at a 1:3000 dilution. Secondary goat anti-mouse antibody conjugated to Alexa Fluor 488 dye (Invitrogen) and Hoescht Dye (nuclear staining) were used at a 1:3000 dilution to detect RSV protein and cell nuclei. Following secondary antibody incubation, the monolayers were washed and left in 100 μL PBS for imaging and quantitation using a Cellomics ArrayScan HCS instrument.

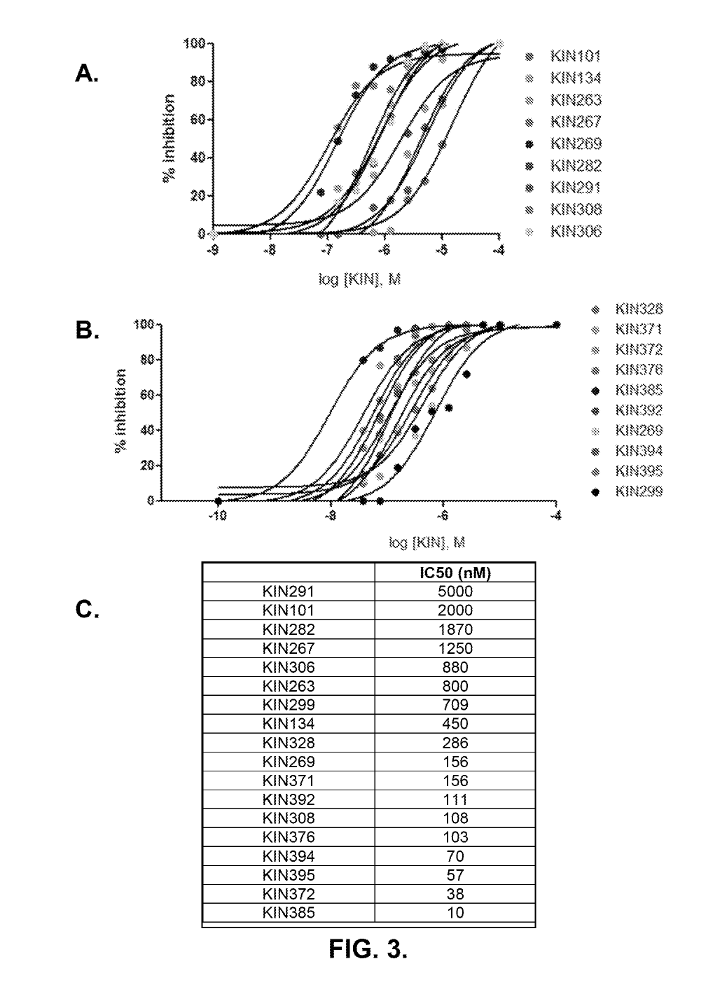

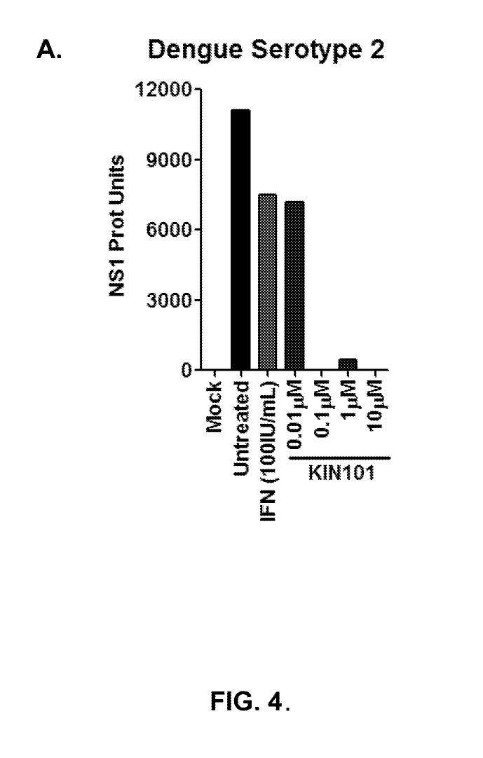

FIGS. 3A, 3B, and 3C show the decrease in foci graphed as percent inhibition of viral infection by compound. KIN101 showed dose-dependent decreases in viral infection of 293 cells; compounds KIN134, KIN263, KIN267, KIN269, KIN282, KIN291, KIN308, and KIN306 improved on this antiviral activity as shown by decreased viral titer. (FIG. 3A). KIN328, KIN371, KIN372, KIN376, KIN385, KIN392, KIN269, KIN394, KIN395, and KIN299 showed dose-dependent decreases in viral infection of 293 cells (FIG. 3B). FIG. 3C shows IC50 values of example selected compounds in the influenza antiviral assay.

Antiviral activity against DNV in vitro was measured for KIN269 and other selected compounds. Cultured human Huh7 cells were seeded in 6-well tissue-culture plates at a density of 4×10⁵ cells per well for the DNV focus-forming assay and grown for 24 hours. Cells were infected with DNV type 2 strain at a MOI of 0.1 for 2 hours and then removed. Compound dilutions were prepared in 0.5% DMSO and used to treat cells at final concentrations of compound ranging from 0.001 to 10 μM per well. Vehicle control wells contained 0.5% DMSO and were used to compare to compound-treated cells. Replication was then allowed to proceed for 48 hours. Virus supernatants were then harvested and used to infect new monolayers of permissive Vero cells that were seeded 24 hours prior in 96-well tissue-culture plates at a density of 8×10³ cells per well. The newly infected cells were incubated for 24 hours and used to measure the level of infectious virus in the original supernatants by immunofluorescent staining of viral protein. The cells were fixed with ice-cold 1:1 methanol and acetone solution and stained for DNV fusion protein. Primary mouse monoclonal antibody against DNV fusion protein (Millipore) was used at a 1:2000 dilution. Secondary goat anti-mouse antibody conjugated to Alexa Fluor 488 dye (Invitrogen) and Hoescht Dye (nuclear staining) were used at a 1:3000 dilution to detect DNV protein and cell nuclei. Following secondary antibody incubation, the monolayers were washed and left in 100 μL PBS for imaging and quantitation using a Cellomics ArrayScan HCS instrument.

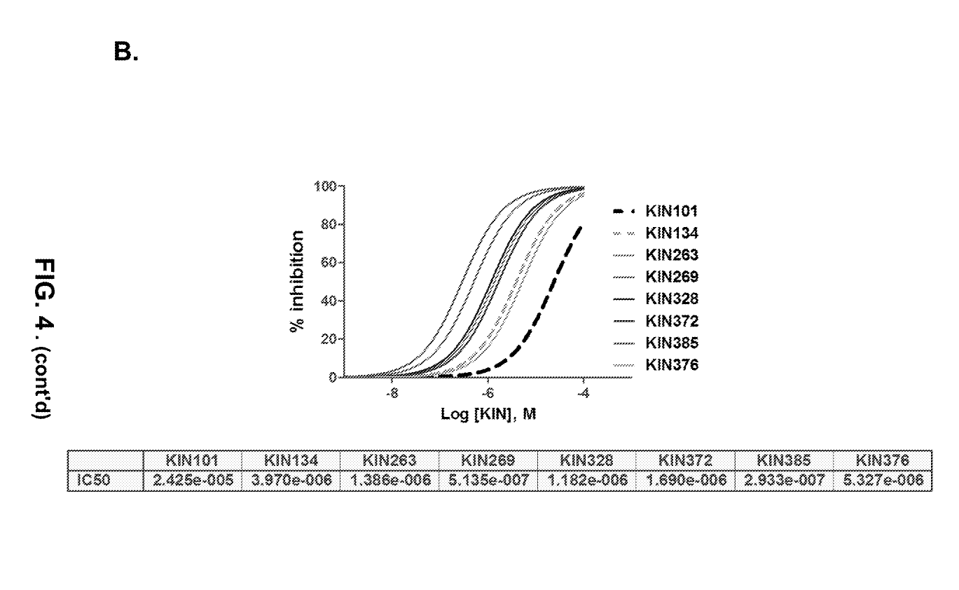

FIG. 4A shows a dose-dependent decrease in viral protein in cells infected with DNV and treated with increasing amounts of KIN101. The results of the DNV focus-forming assay for antiviral activity are shown in FIG. 4B. The decrease in foci is graphed as percent inhibition of viral infection by compound. The compounds KIN101 (black dashed line), KIN134, KIN269, KIN328, KIN372, KIN376, and KIN385 showed dose-dependent decreases in viral infection of Huh7 cells. 1050 values (in M) are shown.

Other virus calculated 1050 values of selected compounds are shown in Table 4.

TABLE 4
IC50 values of selected lead compounds
in example in vitro virus systems.

| | Flu IC50 (μM) | DNV IC50 (μM) |
|---|---|---|
| KIN101 | 2 | >5 |
| KIN134 | 0.45 | 3.97 |
| KIN238 | 1.2 | 1.182 |
| KIN263 | 0.8 | 1.386 |
| KIN269 | 0.145 | 0.542 |
| KIN290 | 1.53 | 3.1 |
| KIN299 | 0.709 | >5 |
| KIN306 | 0.88 | 2.81 |
| KIN308 | 0.108 | 4.16 |
| KIN328 | 0.286 | 1.34 |
| KIN371 | 0.156 | >5 |
| KIN372 | 0.037 | 1.69 |
| KIN376 | 0.103 | 5 |
| KIN378 | 0.6 | >5 |
| KIN385 | 0.009 | 0.293 |
| KIN389 | 0.2 | >5 |
| KIN392 | 0.009 | 0.65 |
| KIN394 | 0.07 | 0.94 |
| KIN395 | 0.057 | 1.97 |
| KIN807 | 0.143 | 0.13 |
| KIN814 | 0.062 | 0.187 |
| KIN823 | 0.153 | 0.371 |
| KIN824 | 0.076 | 0.217 |
| KIN826 | 0.014 | 0.196 |
| KIN844 | 0.002 | 0.316 |
| KIN848 | 0.004 | 0.258 |
| KIN850 | 0.002 | 1.35 |
| KIN851 | 0.417 | 1.187 |
| KIN857 | 0.065 | 0.182 |
| KIN861 | 0.405 | >5 |
| KIN865 | 0.234 | 0.176 |
| KIN866 | 0.027 | 0.507 |
| KIN867 | 0.007 | 0.304 |
| KIN882 | 0.03 | 0.216 |

### Example 7

### In Vitro Antiviral Activity of KIN385 and Other Selected Compounds

Antiviral activity against hCMV in vitro was measured. Primary human foreskin fibroblasts (HFF; ATCC) were seeded in 24-well tissue-culture plates at a density of 1.5×10⁵ cells per well and grown for 24 hours. Cells were infected with hCMV AD169 strain (ATCC) at a MOI of 0.1 for 4 hours and then removed. Compound dilutions were prepared in 0.5% DMSO and used to treat cells at final concentrations of compound ranging from 0.001 to 10 μM per well. Vehicle control wells contained 0.5% DMSO and were used to compare to compound-treated cells. Replication was then allowed to proceed for 48-96 hours. Virus supernatants were harvested at 48, 72, and 96 hours and used to infect new monolayers of HFFs that have been seeded 24 hours prior in 96-well tissue-culture plates at a density of 3×10⁴ cells per well. The newly infected cells were incubated for 24 hours and used to measure the level of infectious virus in the original supernatants by immunofluorescent staining of viral protein. The cells were fixed with ice-cold 1:1 methanol and acetone solution and stained for hCMV 1E1 protein similarly to previously described methods for the other in vitro virus systems.

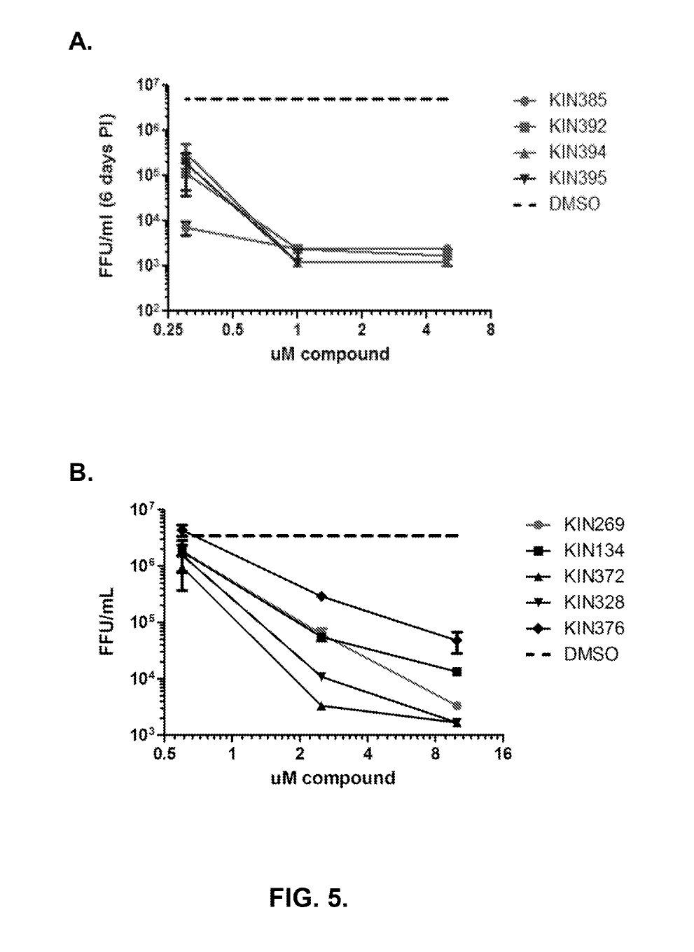

FIG. 5A shows dose-dependent decreases in hCMV as measured by foci (FFU/mL) in samples treated with KIN385, KIN392, KIN394, and KIN395. FIG. 4B shows dose-dependent decreases in hCMV as measured by foci (FFU/mL) in samples treated with KIN269, KIN134, KIN372, KIN328, and KIN376.

### Example 8

### In Vitro IRF-3 Activation by KIN269

RIG-I signaling pathway activation by KIN269 was measured by assaying activation of IRF-3 dependent signaling. This was done by measuring IRF-3 dependent gene expression by RT-qPCR in cells treated with compound. Cultured human cells were treated with 0.001-10 μM of KIN269 or DMSO vehicle control and incubated for up to 24 hours. Cells are harvested at time points from 4-24 hours after treatment. RNA isolation, reverse transcription, and qPCR were performed using well known techniques. PCR reactions were performed using commercially available, validated TaqMan gene expression assays (Applied Biosystems/Life Technologies) according to manufacturer instructions. Gene expression levels were measured using a relative expression analysis (ΔΔCt).

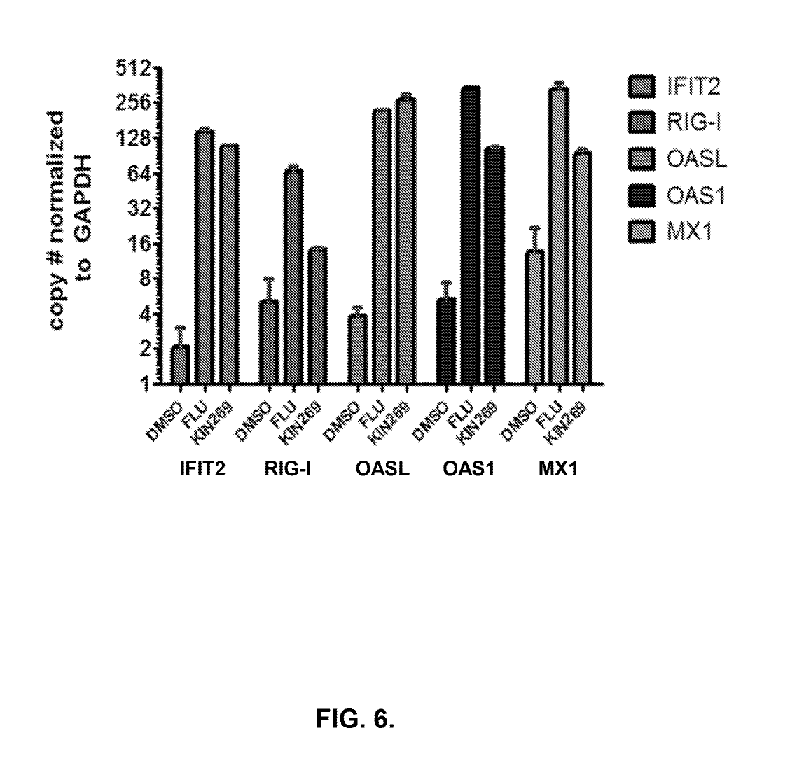

FIG. 6 shows induction of gene expression by the compound KIN269 in 293 cells. Genes known to be IRF-3 dependent or involved in the antiviral response are shown to be induced after treatment with KIN269.

### Example 9

### In Vitro Bioavailability and Antiviral Activity of KIN269

Antiviral activity of KIN269 was measured using a mouse influenza model. Virus infection was achieved with non-surgical instillation of influenza virus strains A/Puerto Rico/8/1934 (PR8). KIN269 was administered daily by intranasal administration of 10 mg/kg in 10% hydroxypropyl-β-cyclodextrin (HPBCD) or vehicle-only control over the entire course of infection. Animals were evaluated for study endpoints including daily clinical observations, mortality, body weight, and body temperature. Virus titer was measured in lung tissue.

Antiviral activity of KIN269 was measured using a mouse coronavirus (MHV) model. Virus infection was achieved using non-surgical intranasal instillation of MHV. KIN269 was administered daily by intranasal administration of 10 mg/kg in 10% hydroxypropyl-β-cyclodextrin (HPBCD) or vehicle-only control over the entire course of infection. Animals were evaluated for study endpoints including daily clinical observations, mortality, body weight, and body temperature. Virus titer was measured in lung tissue.

Antiviral activity of KIN269 was measured using a mouse DNV model. Virus infection is achieved using intraperitoneal injection of DNV type 2 strain. KIN269 was administered daily by IP injection of 10 mg/kg or vehicle-only control over the entire course of infection. Animals were evaluated for study endpoints including daily clinical observations, mortality, body weight, and body temperature. Virus RNA was measured in serum.

In a preliminary mouse PK study, 10 mg/kg KIN269 was administrated by both an intravenous and an intraperitoneal route of administration. Blood samples were collected by retro-orbital sinus prior to dosing and at time points up to 4 hours post dosing. Compound concentrations were measured according to a developed bioanalytical method specific to KIN269.

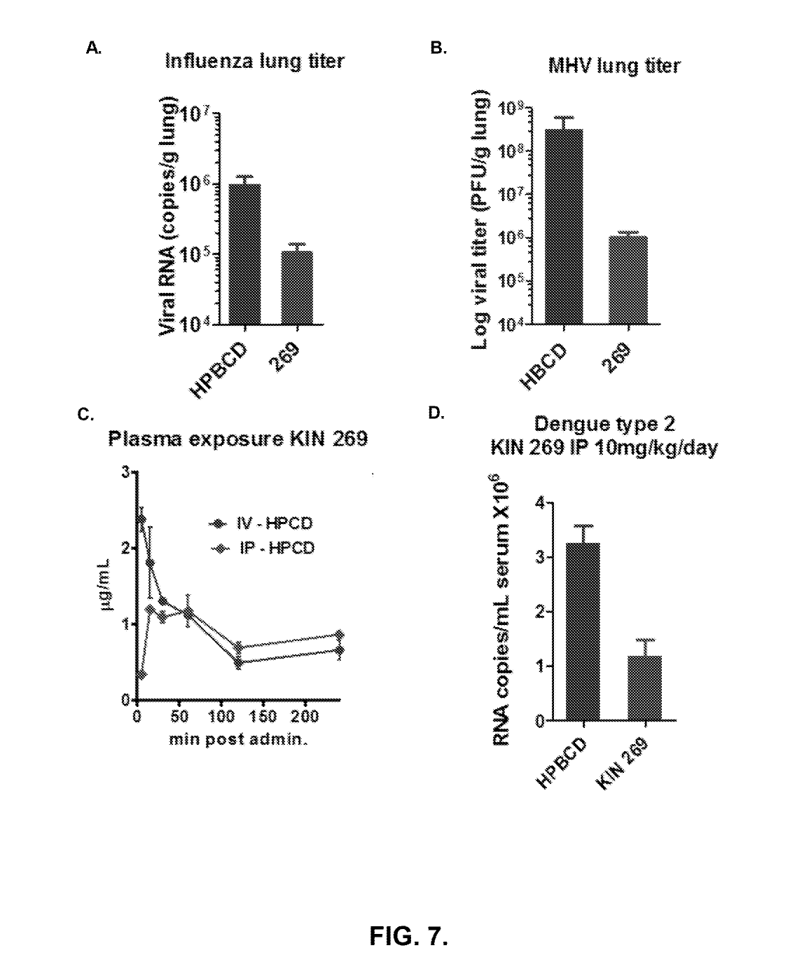

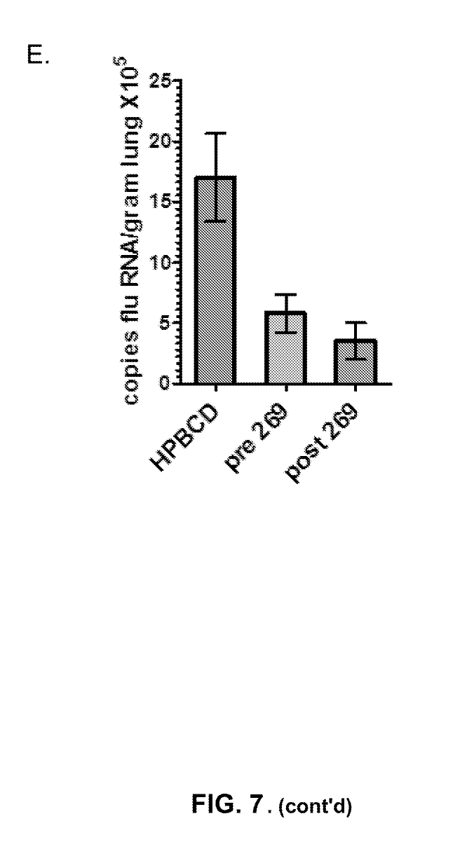

FIGS. 7A-7E show in vivo broad spectrum antiviral activity and bioavailability of KIN269. KIN269 (10 mg/kg in 10% HPBCD) intranasal treatment reduces replication and titer of influenza virus (FIG. 7A) and mouse hepatitis virus (MHV) (FIG. 7B) in the lung. FIG. 7C shows KIN269 serum levels over time when dosed at 10 mg/kg via intraperitoneal injection or intravenous injection. FIG. 7D shows that KIN269 inhibited DNV as measured in serum when dosed IP 10 mg/kg/day. FIG. 7E shows that KIN269 (20 mg/kg) inhibited flu replication in the lung when administered by intranasal instillation either −24 hours prior (prophylactic) or +24 hours post (therapeutic) lethal infection with PR8 flu. Lung tissue was harvested 72 hours after infection and flu RNA was quantitated by PCR.

### Example 10

### Antiviral Activity and Pharmacological Properties Using Quantitative Structure-Activity Relationship (QSAR) Studies

This Example describes analog compound design using QSAR approach of the compounds described herein for antiviral action. The QSAR studies are designed to provide lead compounds with picomolar to nanomolar potency. Optimization of the compounds focuses on creating structural diversity and evaluating core variants and group modifications. Analogs are tested for antiviral activity against several viruses including the virus assay models described herein. Furthermore, analogs are tested for cytotoxicity in one or more cell lines or peripheral blood mononuclear cells. Optimized compounds that show improved efficacy and low cytotoxicity are further characterized by additional measures of in vitro and in vivo toxicology and absorption, distribution, metabolism, and elimination (ADME). Their mechanism of action and breadth of antiviral activity are also studied.

Chemical design in QSAR studies. Analysis of drug-like properties, metabolic lability, and toxic potential is performed in order to drive analog compound design. Drug-like properties, as measured by Lipinski's Rules (Lipinski, C. A., et al. (2001) Experimental and computational approaches to estimate solubility and permeability in drug discovery and development settings, Adv Drug Deliv Rev 46, 3-26), and related physiochemical properties are primary indicators of bioavailability. Structural features that suggest metabolic and toxicological liabilities may indicate limited stability, reduced half-life, reactive intermediates, or idiosyncratic toxicity and will therefore be removed. A 5- to 10-compound analog set is constructed to remove or alter chemically reactive or metabolically susceptible structural features, thereby developing a preliminary QSAR.

The compounds disclosed herein are described as isoflavone compounds. Isoflavones are best known as natural products isolated from the Leguminosae (legume) family and are usually polyhydroxylated and pharmacologically active as phytoestrogenics and antioxidants. The most recognizable member of this class is genistein, which has been reported to have anticancer activities and to induce thymic and immune changes in mammals (Banerjee, S., et al. (2008) Multi-targeted therapy of cancer by genistein, Cancer Lett 269, 226-242). It is relevant that a preliminary screen of a Natural Cancer Institute (NCI) natural product library revealed genistein as a validated hit for interferon-stimulated gene (ISG) induction. This correlation demonstrates the potential for broad flexibility in functional group modifications and analog design while retaining biological activity.

For each analog, a (high-performance liquid chromatography) HPLC- and/or HPLC-mass spectrometry-based analytical method is used to evaluate compound and metabolite concentrations in various test systems. Although the specific analytical method is optimized for each compound, reverse-phase chromatography can be used alone or in combination with quadrupole mass spectrometry to characterize the identity and purity of several of the lead compounds. Initially, compound stability over time in increasing concentrations of serum, plasma, and whole blood from mammalian species (such as mouse, cynomolgus macaque, and human) will be evaluated by HPLC, and a half-life will be determined. In some instances, prominent metabolites are characterized by mass spectrometry.

### Example 11

### In Vitro Biological Activity

Compounds described herein, including the compounds listed in Table 1, are tested for biological activities including: activation of target pathways including immune response pathways, antiviral activity against a variety of viruses, low cytotoxicity, and a therapeutic index greater than 10.

RIG-I signaling pathway activation by compounds. One example of an assay to measure RIG-I pathway activation is the measurement of downstream gene expression by RT-qPCR in cells treated with compound. The transcription factor IRF-3 is activated through RIG-I signaling and the increased expression of IRF-3 dependent genes indicate activation of the RIG-I pathway. Other genes that are associated with the host innate immune antiviral response are also measured as indicators of compound activity.

Cultured human cells are treated with 0.001-10 μM of compound or a DMSO vehicle control and incubated for up to 24 hours. Cells are harvested at time points from 4-24 hours after treatment. RNA isolation, reverse transcription, and qPCR are performed using well known techniques. PCR reactions are performed using commercially available, validated TaqMan gene expression assays (Applied Biosystems/Life Technologies) according to manufacturer instructions. Gene expression levels are measured using a relative expression analysis (ΔΔCt).

Gene expression can be similarly assayed in cell types that include: primary blood mononuclear cells, human macrophages, THP-1 cells, Huh7 cells, A549 cells, MRC5 cells, rat splenocytes, rat thymocytes, mouse macrophages, mouse splenocytes, and mouse thymocytes. Expression of other genes of interest can be assayed as described herein. In addition, gene expression can be assayed in the presence of virus in order to determine compound activity in the context of active viral infection.

Innate immune response induction by compounds. The activity of compounds can be assayed in primary immune cells to determine whether compound treatment stimulates immune response pathways. One example is to assay cytokine expression in cultured human primary blood cells such as dendritic cells. Cells are seeded in tissue culture dishes and treated with compound ranging from 0.001-10 μM of compound. For assay of cytokine production, supernatants from treated wells are isolated 24-48 hours after compound treatment and tested for levels of cytokine protein. Cytokines are detected using specific antibodies conjugated to magnetic beads and a secondary antibody that reacts with Streptavidin/Phycoerythrin to produce a fluorescent signal. The bound beads are detected and quantified using the MAGPIX® (Luminex Corp.) instrument, although similar techniques as are known in the art may be used to measure fluorescent protein production, such as for example an ELISA.

Other cells from which cytokine secretion can be measured include, for example human peripheral blood mononuclear cells, human macrophages, mouse macrophages, mouse splenocytes, rat thymocytes, and rat splenocytes.

Cytotoxicity is evaluated using standard in vitro assays including MTS assay and caspase assay. Protocols to perform these assays are known to those skilled in the art and there are several commercially available kits to measure assay readout, such as a colorimetric based assay to measure conversion of MTS to formazan (Cell Titer One, Promega) and a sandwich ELISA based assay to measure levels of activated caspase-3 (PATHSCAN® Cleaved Caspase-3 (Asp175) Sandwich ELISA Kit #7190, Cell Signaling Technology, Inc., Danvers, Mass.). Cultured human cells are treated with increasing amounts of compound from 0 up to at least 50 μM or equivalent amounts of DMSO diluted in media to evaluate their effect on cell viability. Cultured human cell lines that are used in this assay include Huh7, PH5CH8, A549, or HeLa cells.

In vitro pharmacology and toxicology. This description of toxicological assays is exemplary. In vitro studies are performed to measure performance of the most promising analogs in one or more assays of intestinal permeability, metabolic stability, and toxicity. These studies can include plasma protein binding; serum, plasma, and whole-blood stability in human and model organisms; intestinal permeability; intrinsic clearance; human Ether-àa-go-go (hERG) channel inhibition to test potential cardiac toxicity; and genotoxicity using for example a reversion mutation assay (Ames test) and/or a micronucleus formation assay. Human plasma protein binding will be evaluated by partition analysis using equilibrium dialysis. For intestinal permeability modeling, apical-to-basolateral flux is assessed in a human epithelial cell line such as Caco-2 or TC7. Hepatic clearance is estimated for a subset of the most promising analogs by measuring the rate of disappearance of the parent compound during incubation in human liver microsomes. Specific metabolites may be isolated and characterized.

### Example 12

### Assays of Antiviral Activity Using In Vitro Models

The compounds disclosed herein have efficient activity against several viruses in vitro. To further characterize the breadth of antiviral activity of optimized compounds, cell culture infection models are used to analyze different viruses as well as different strains of the same virus (Table 4). Assays to measure the antiviral activity of compounds against several of these viruses is described herein.

The studies include treating cells with compound 2-24 hours prior to infection and/or treating cells 2-8 hours after infection. Compound is administered at different concentrations ranging from 0.001-10 μM. Positive control treatments used include interferon, ribavirin, oseltamivir, or other known treatment to inhibit the infection of the specific virus. Virus production and cellular ISG expression are assessed over a time course to analyze antiviral activity of each compound (Table 4). Virus production is measured by focus-forming or plaque assay.

An immunofluorescent based focus-forming assay is performed in cultured human HeLa cells to measure antiviral activity against RSV. Experimental conditions are as or substantially similar to those described in Example 5.

Antiviral activity against influenza virus in vitro is measured by immunofluorescent based focus-forming assay. Influenza A virus strains that are used in this assay include A/Udorn/72 H3N2 strain and A/California/04/09 H1N1 strain. Experimental conditions are as or substantially similar to those described in Example 6.

Antiviral activity against DNVs in vitro is measured by immunofluorescent based focus-forming assay. Experimental conditions are as or substantially similar to those described in Example 6.

Antiviral activity against hCMV in vitro is measured by immunofluorescent based focus-forming assay. Experimental conditions are as or substantially similar to those described in Example 7.

In parallel experiments, viral RNA and cellular ISG expression are measured by qPCR and immunoblot analyses. These experiments are designed to validate compound signaling actions during virus infection, and assess compound actions to direct innate immune antiviral programs against various strains of viruses and in the setting of virus countermeasures. Detailed dose-response analyses of each compound are conducted in each virus infection system to determine the effective dose that suppresses virus production by 50% (1050) and 90% (1090) as compared with control cells for both the pre-treatment and post-treatment infection models.

The broad spectrum antiviral activity of selected compounds are shown in FIGS. 2A and 2B (RSV); FIGS. 3A, 3B, and 3C (flu); FIGS. 4A and 4B (DNV); and FIGS. 5A and 5B (hCMV).

Infection models that can be assayed by in vitro assays include WNV, HBV, EMCV, and SARS.

TABLE 5
Exemplary virus systems and study design for antiviral analysis

| Virus | Virus Strain | Study Design |
|---|---|---|
| HCV | H77 (genotype 1a) | Assays |
| | JFH1 (genotype 2a) | Plaque or focus forming |
| RSV | A2 long strain | assays |
| FLU | High pathogenicity in mice | (infectious virus) |
| | A/PR/8/34 (H1N1 mouse-adapted | qPCR (RNA levels) |
| | virus) | Immunoblot and ELISA |
| | A/WSN/33 (H1N1 mouse-adapted | (protein levels) |
| | neurovirulent virus) | Study Design |
| | Low pathogenicity in mice | Compound treatment of cells |
| | A/Texas/36/91 (H1N1 | pre- and post-infection |
| | circulating virus) | Determine IC50 and IC90 |
| | A/Udorn/72 (H3N2) | Inhibition of viral life |
| | A/California/07/09(H1N1) | cycle |
| DNV | Type 2 | |
| WNV | TX02 (lineage 1) | |
| | MAD78 (lineage 2) | |

### Example 13

### In Vivo Pharmacokinetic and Toxicological Profiles of Optimized Compounds in Preclinical Animal Models

Preclinical pharmacokinetic (PK) and tolerability profiling. The in vivo PK profile and tolerability/toxicity of optimized compounds are evaluated in order to conduct further characterization of their antiviral activity in animal models of virus infection. Mouse and rat are the chosen test species for these studies because there are several established virus models in the mouse and models of PK, toxicology, and immunology in the rat.

Reverse-phase, HPLC-MS/MS detection methods are used to detect and quantify the concentration of each compound in biological samples including plasma and target tissue samples. Prior to PK profiling, an initial oral and injectable pharmaceutical composition for each compound is developed using a limited pharmaceutical composition component screen that is largely focused on maximizing aqueous solubility and stability over a small number of storage conditions. Existing analytical methods known in the art are used to measure pharmaceutical composition performance. A pharmaceutical composition is developed for each compound following a three tiered strategy. Tier 1: pH (pH 3 to 9), buffer, and osmolality adjustment; Tier 2: addition of ethanol (<10%), propylene glycol (<40%), or polyethylene glycol (PEG) 300 or 400 (<60%) co-solvents to enhance solubility; Tier 3: addition of N—N-dimethylacetamide (DMA, <30%), N-methyl-2-pyrrolidone (NMP, <20%), and/or dimethyl sulfoxide (DMSO, <20%) co-solvents or the cyclodextrins (<40%) as needed to further improve solubility.

In preliminary mouse PK studies, the following criteria are evaluated after compound has been administrated by at least 2 routes of administration including orally and i.v.: oral bioavailability, Cmax, t_{1/2}, CI, Vd, AUC0-24,0-∞. Each compound is administered as a single dose to animals by oral gavage (up to 10 mg/kg) or intravenous bolus injection (up to 5 mg/kg) after an overnight fast. Multiple animals are dosed for each dosing group such that 3 animals can be sampled at each time point. Blood samples are collected by retro-orbital sinus prior to dosing and at 5, 15, and 30 min, and 1, 2, 4, 8, and 24 hours post dosing. Target tissues, including lung, liver, and lymph nodes, are also collected at the time point of final blood collection. Compound concentrations are measured according to the previously developed bioanalytical method. PK parameters are evaluated using the WinNonlin software.

Based upon performance in exploratory PK studies, compounds are further evaluated for preliminary tolerability and toxicity in mice prior to their characterization in antiviral models. Tolerability studies are performed in two stages: an initial dose escalation stage that includes ascending doses up to 5 doses, each separated by a 5-day washout period, to determine the maximum tolerable dose (MTD; Stage 1); this is followed by seven daily administrations of the MTD to evaluate acute toxicity (Stage 2). In the tolerability study, all doses are administered by oral gavage. In such an experiment, five animals of each sex are placed on-study in stage 1 and 15 animals per sex per dosing group in Stage 2. Study endpoints include a determination of the MTD, examination for acute toxicity, physical examination, clinical observations, hematology, serum chemistry, and animal bodyweights. Gross pathology is performed on all animals whether found dead, euthanized in extremis, or at the intended conclusion of the experiment. The toxicology studies are primarily exploratory in nature and intended to identify early toxicological endpoints, and drive selection of lead compounds for antiviral animal models.

### Example 14

### In Vivo Antiviral Properties of Optimized Compounds in Preclinical Animal Models

This Example describes the evaluation of antiviral properties and immune protection using mouse infection models. Optimized compounds are selected based on compound PK, antiviral, and innate immune actions for further evaluation in preclinical mouse models of infection. Innate immune actions of the compounds are measured, and their ability to protect mice from WNV and influenza virus challenge is assessed. For the WNV infection model, subcutaneous footpad infection of wild-type C57Bl/6 mice with the virulent lineage 1 strain of WNV (WNV-TX) are performed (Suthar, M. S., et al. (2010). IPS-1 is essential for the control of WNV infection and immunity, PLoS Pathog 6, e1000757). Non-surgical tracheal instillation is performed for influenza virus strains A/PR/8/34, A/WSN/33, and A/Udorn/72.

The influenza virus strains in these experiments include at least two different subtypes (for example, H1N1 and H3N2) and exhibit varying pathogenic properties and clinical presentations in C57Bl/6 mice (Barnard, D. L. (2009) Animal models for the study of influenza pathogenesis and therapy, Antiviral Res 82, A110-122). Mice are monitored for morbidity and mortality over a range of challenge doses (such as, 10 to 1,000 pfu of virus) either alone or in combination with compound treatment beginning up to 24 hours before or up to 24 hours after infection and continuing daily subject to the determined plasma half-life of the compound. Compound dose-response analysis and infection time course studies are conducted to evaluate compound efficacy to: 1) limit serum viral load; 2) limit virus replication and spread in target organs; and 3) protect against viral pathogenesis.

For WNV, in addition to serum, viral burden is assessed in lymph nodes, spleen, and brain; for influenza virus, viral burden is assessed in heart, lung, kidney, liver, and brain. Incorporated in the design of these experiments is the determination of an effective dose for 50% and 90% suppression of serum viral load (ED50 and ED90) by each compound after a standard challenge of 100 pfu of WNV-TX or 1,000 pfu of influenza virus. Serum viral loads are determined by qPCR of viral RNA at 24 hour intervals following compound treatment. The compound actions are tested at the ED50 and ED90 toward limiting WNV pathogenesis in the cerebral nervous system using a WNV neuroinvasion model of infection (Daffis, S., et al. (2008) Toll-like receptor 3 has a protective role against West Nile virus infection, J Virol 82, 10349-10358). Mice are monitored for morbidity and mortality after standard intracranial challenge of 1 pfu of WNV-MAD, either alone or in combination with compound treatment beginning 24 hours after infection.

For these and other in vivo virus infection models, the compound (or pharmaceutical composition, as appropriate) can be administered via routes including oral, nasal, mucosal, intravenous, intraperitoneal, subcutaneous, or intramuscular. Other in vivo virus infection models that can used to evaluate compound antiviral activity include SARS, DNV, MCMV, or EMCV.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. As used herein, a material effect would cause a statistically significant reduction in a disclosed compound's or pharmaceutical composition's ability to treat a viral infection in a subject; reduce viral protein in a subject or assay; reduce viral RNA in a subject or assay or reduce virus in a cell culture.

Unless otherwise indicated, all numbers used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to publications, patents and/or patent applications (collectively “references”) throughout this specification. Each of the cited references is individually incorporated herein by reference for their particular cited teachings.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A compound having a structure 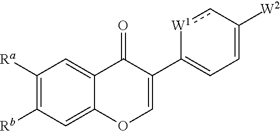
wherein,
W¹ is CH, CH₂, N, or NH;
W² is Br, Cl, F, phenyl, CF₃, lower alkyl, C(CH₃)₃, heteroaryl, cycloalkyl, OW^{a}, OCH₂W^{a}, OCH₂W^{b}, NHSO₂W^{b}, or NW^{c}SO₂W^{c};
W^{a} is Br, aryl, CF₃, lower alkyl, cycloalkyl, heterocycloalkyl, CHF₂, C(CH₃)₃, or NHSO₂W^{b};
W^{b} is phenyl, cycloalkyl, heterocycloalkyl, or lower alkyl;
W^{c} is lower alkyl;
R^{a} is H, lower alkyl or OR^{c}, where R^{c} is H or lower alkyl;
R^{b} is phenyl, phenol, OR^{d}, NR^{d}, OR^{d}R^{e}, or NR^{d}R^{e};
R^{d} is lower alkyl, alkylsulfonyl, SO₂CH₃, alkylcarbonyl, CF₂, C(═O)NHR^{c}, CH₂C(═O)R^{f}, CH₂C(═O)R^{f}R^{g}, CH₂R^{h}, CH₂CH₂R^{f}, CH₂CH₂R^{f}R^{g}, or CH₂CH₂R^{f}Rⁱ;
R^{e} is hydroxyl, lower alkyl, alkylsulfonyl, or NHR^{c};
R^{f} is heteroaryl or heterocycloalkyl,
R^{g} is alkylcarbonyl, alkylsulfonyl, or lower alkyl;
R^{h} is alkynyl; and
the dashed lines represent the presence or absence of a double bond.

2. A compound of claim 1, wherein W² is Br, CF₃, OCF₃, or C(CH₃)₃ and R^{b} is OR^{j}, where R^{j} is sulfonyl.

3. A compound of claim 1, wherein W₂ is C(CH₃)₃ and R^{b} is NCH₃R^{j}, where R^{j} is sulfonyl.

4. A compound having a structure: 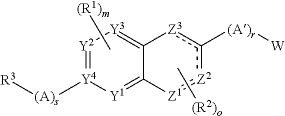
wherein,
R¹ and R² are each independently selected from H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidyl, N-alkyl piperizinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, SR⁴, SOR⁴, SO₂R⁴, CO₂R⁴, COR⁴, CONR⁴R⁵, CH₂CONR⁴R⁵, NR⁴SO₂R⁵, CSNR⁴R⁵, or SOₘNR⁴R⁵;
R³ is H, R¹, alkylsulfonyl, NR⁴SO₂R⁵, SOₘNR⁴R⁵, lower alkyl, aryl, alkenyl, alkynyl, haloalkyl, alkylaryl, arylalkyl, alkoxyalkylaryl, alkylamino, arylamino, heteroalkyl, heteroaryl, cyclic heteroalkyl, acyl, arylsulfonyl, or heterocyclicalkylalkyl;
R⁴ and R⁵ are each independently selected from H, lower alkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO₂, OCF₃, CF₃, Br, Cl, F, 1-amidino, 2-amidino, alkylcarbonyl, morpholino, piperidyl, N-alkyl piperizinyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, or isoquinoline;
A and A′ are each independently selected from O, S, or NR′, where R′ is H, lower alkyl or R³, or R′ and R³ or R′ and W can come together to form an unsubstituted or substituted heterocyclic ring or heteroaryl ring;
W is aryl, substituted aryl, heteroaryl, substituted heteroaryl, alkyl, substituted alkyl, cycloalkyl, substituted cycloalkyl, heteroalkyl, substituted heteroalkyl, heterocycloalkyl, substituted heterocycloalkyl, arylalkyl, or heteroaryl alkyl;
Z¹, Z², and Z³ are each independently selected from C, O, NH, S, C═O, S═O, or SO₂;
Y¹, Y², Y³, and Y⁴ are each independently selected from C or N, provided that when Y⁴ is N, then R³-(A)s is not present;
the dashed lines represent the presence or absence of a double bond;
m is 1 or 2;
n is 0, 1, 2 or 3;
o is 0, 1, 2, or 3;
s is 0 or 1; and
r is 0 or 1.

5. A compound of claim 4, wherein the compound has a structure

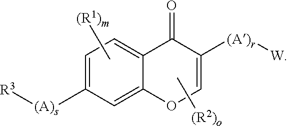

6. A compound of claim 4, wherein Y⁴ is N.

7. A compound of claim 4, wherein W has a structure selected from: 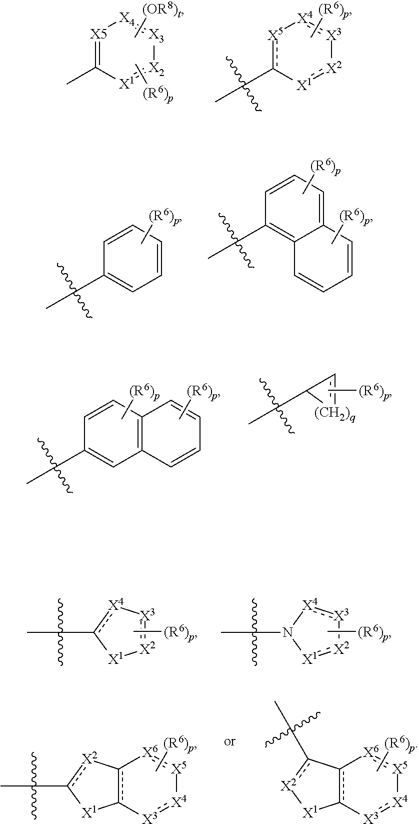
wherein,
each of X¹, X², X³, X⁴, X⁵, and X⁶ are independently selected from C, O, NH, NR⁶, S, C═O, S═O, or SO₂;
each R⁶ is independently selected from H, lower alkyl, haloalkyl, cycloalkyl, aryl, alkenyl, alkynyl, alkylaryl, arylalkyl, alkoxy, aryloxy, arylalkoxy, alkoxyalkylaryl, alkylamino, arylamino, heteroalkyl, heteroaryl, cyclic heteroalkyl, acyl, NH₂, OH, CN, NO2, OCF₃, CF₃, Br, Cl, F, 1-amidino, 2-amidino, alkylcarbonyl, morpholino, piperidyl, dioxanyl, pyranyl, heteroaryl, furanyl, thiophenyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, N-alkyl piperazinyl, quinoline, isoquinoline, SR⁴, SOR⁴, SO₂R⁴, CO₂R⁴, COW, CONR⁴R⁵, NR⁴SO₂R⁵, CSNR⁴R⁵, or SOₘNR⁴R⁵, or two adjacent R⁶ groups can come together to form a fused 5- or 6-membered cycloalkyl ring, heterocycloalkyl ring, methylene dioxo ring, ethylene dioxo ring, aryl ring, or heteroaryl ring;
each R⁸ is independently selected from H, alkyl, haloalkyl, cycloalkyl, aryl, alkenyl, alkynyl, alkylaryl, arylalkyl, alkoxyalkylaryl, heteroalkyl, heteroaryl, cyclic heteroalkyl, acyl, CF₃, alkylcarbonyl, tetrazolo, thiazole, isothiazolo, imidazolo, thiadiazole, thiadiazole S-oxide, thiadiazole S,S-dioxide, pyrazolo, oxazole, isoxazole, pyridinyl, pyrimidinyl, quinoline, isoquinoline, CO₂R⁴, COR⁴, CONR⁴R⁵, SO₂CH₃, or two adjacent R⁸ groups can come together to form a fused 5- or 6-membered cycloalkyl ring, heterocycloalkyl ring, methylene dioxo ring, ethylene dioxo ring, aryl ring, or heteroaryl ring;
p and t are each independently 0, 1, 2, 3, 4, or 5, provided that p+t≦5; and
q is 1, 2, 3, or 4.

8. A compound of claim 7, wherein R⁶ is H, methyl, ethyl, propyl, isopropyl, butyl, sec-butyl, isobutyl, tert-butyl, Cl, Br, CF₃, OCF₃, or —NHSO₂R⁷, where R⁷ is lower alkyl, cycloalkyl, heterocycloalkyl, aryl, or heteroaryl.

9. A compound of claim 8, wherein R⁷ is N-piperidyl, N-morpholino, N-alkyl-N-piperazinyl, or phenyl.

10. A compound of claim 7, wherein:
r is 0 and W is 1-naphthyl, cyclopentyl, 2-thiazolyl, 2-pyrazinyl, 2-benzoxazolyl, or 4-R⁶-1-phenyl and R⁶ is tert-butyl, Br, OCF₃, or —NHSO₂R⁷, where R⁷ is N-piperidyl or phenyl; or
r is 1, and W is phenyl.

11. A compound of claim 7, wherein r is 0 and W is 4-(OR⁸)-1-phenyl and (OR⁸) is trifluoromethoxy, butanyloxy, cyclopropylmethoxy, dimethylpropoxy, trifluoroethoxy, difluoromethoxy, oxanylmethoxy, oxanylmethoxy, or dimethylbutoxy.

12. A compound of claim 4, wherein:
s is 1, A is O or NR′ where R′ is H or lower alkyl, and R³ is H, 3-propynyl, SO₂CH₃, CF₂H, CF₃, CONHCH₃, or CH₂CONR⁴R⁵; where R⁴ and R⁵ come together to form a morpholino ring, an N-acetyl piperazinyl ring, an N-methanesulfonyl piperazinyl ring, or an N-methyl piperazinyl ring; or
s is 0 and R³ is SO₂CH₃, COR⁴, CONR⁴R⁵, N-imidazolinyl, or N-maleimido.

13. (canceled)

14. A pharmaceutical composition comprising a compound of claim 1.

15. A pharmaceutical composition of claim 14, for use in therapy.

16. A pharmaceutical composition of claim 14, for use in treating or preventing a viral infection in a subject.

17. A pharmaceutical composition for use in therapy, comprising a compound having a structure selected from:

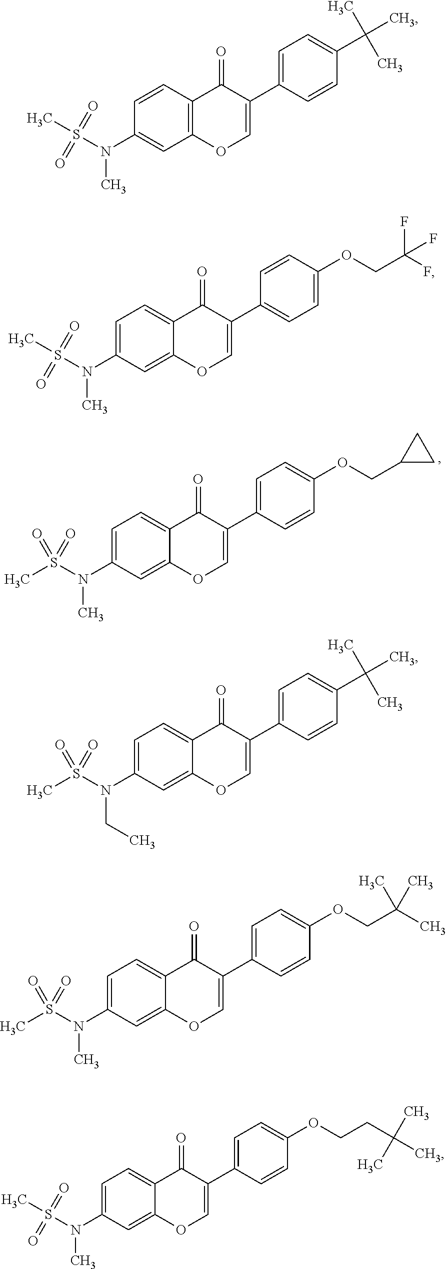

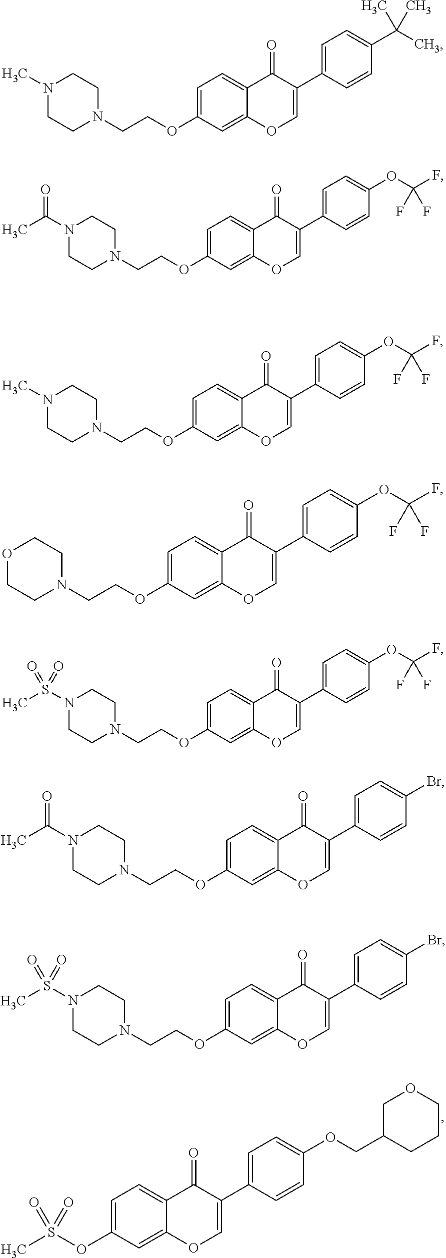

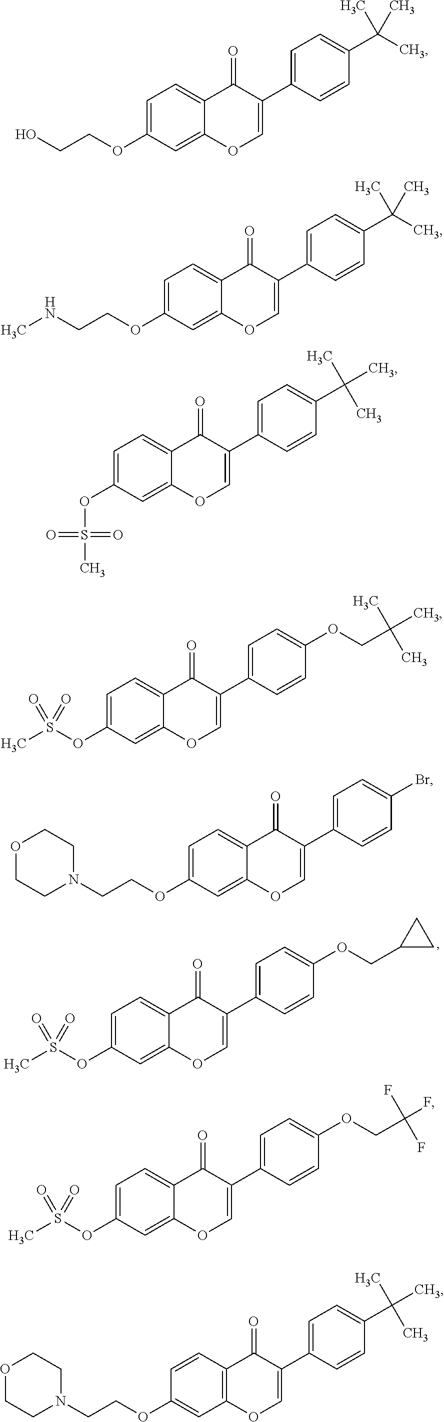

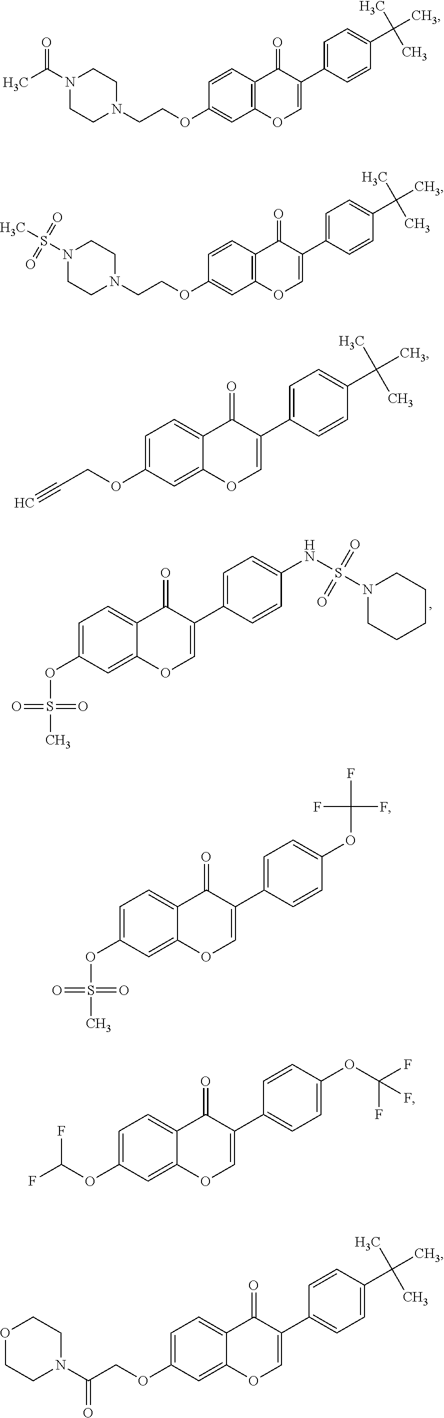

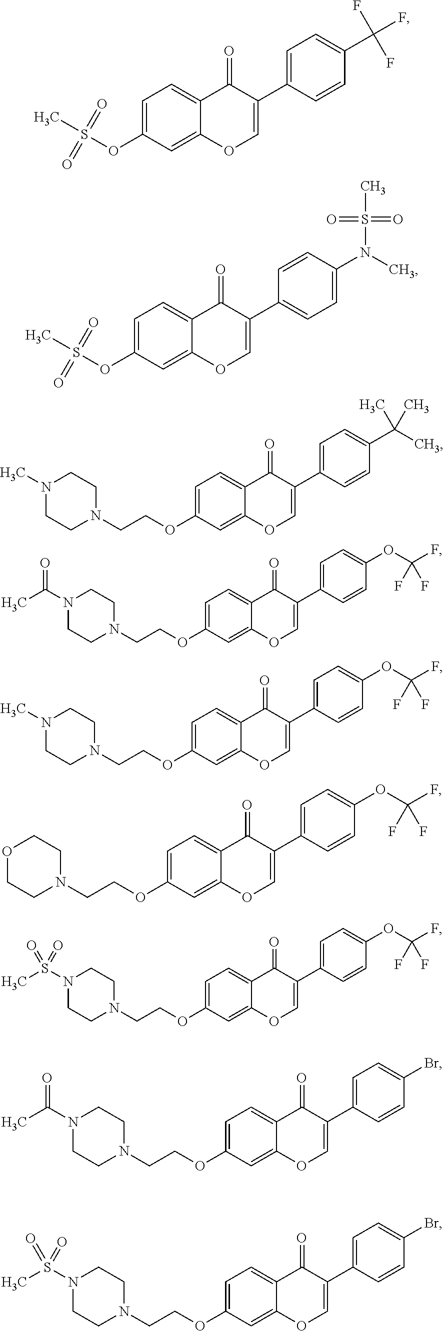

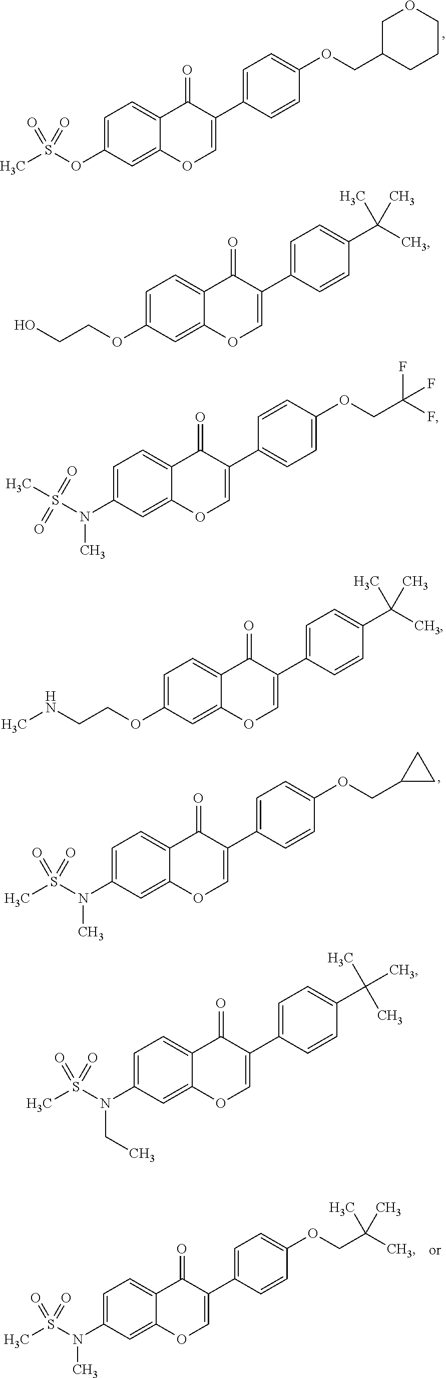

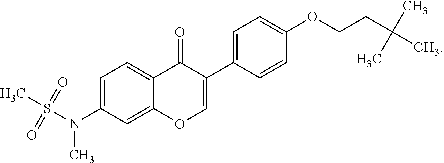 [SMILES: CN(c1ccc2c(=O)c(-c3ccc(OCCC(C)(C)C)cc3)coc2c1)S(C)(=O)=O]

18. A pharmaceutical composition for use according to claim 15, wherein said pharmaceutical composition is administered as an adjuvant for a prophylactic or therapeutic vaccine.

19. A compound of claim 1 for use in modulating an innate immune response in a eukaryotic cell, the use comprising administering the compound to the eukaryotic cell.

20. A compound for use in modulating an innate immune response in a eukaryotic cell, the use comprising administering the compound to the eukaryotic cell, wherein the compound has a structure selected from:

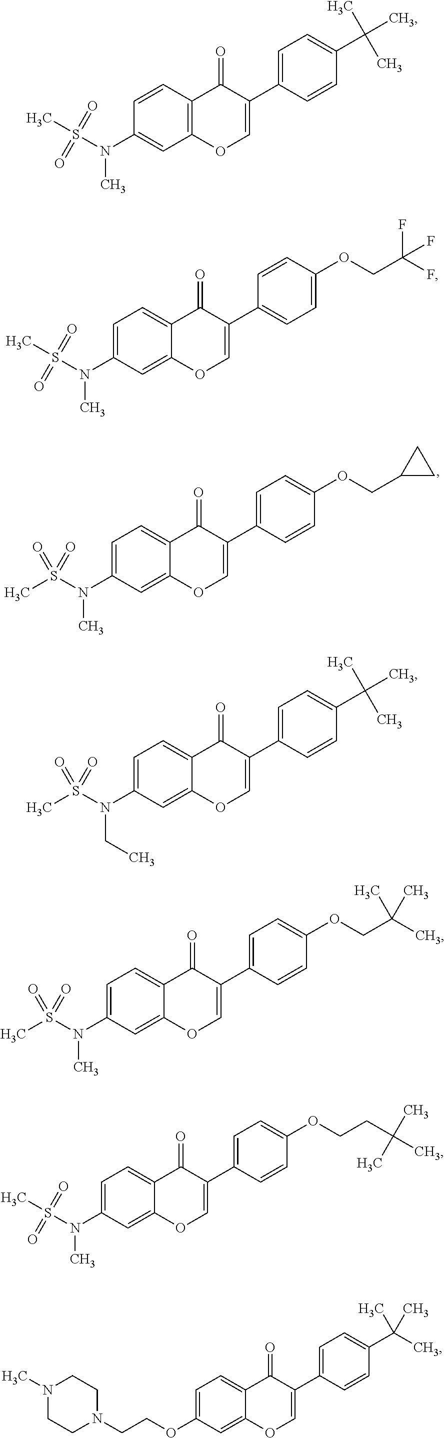

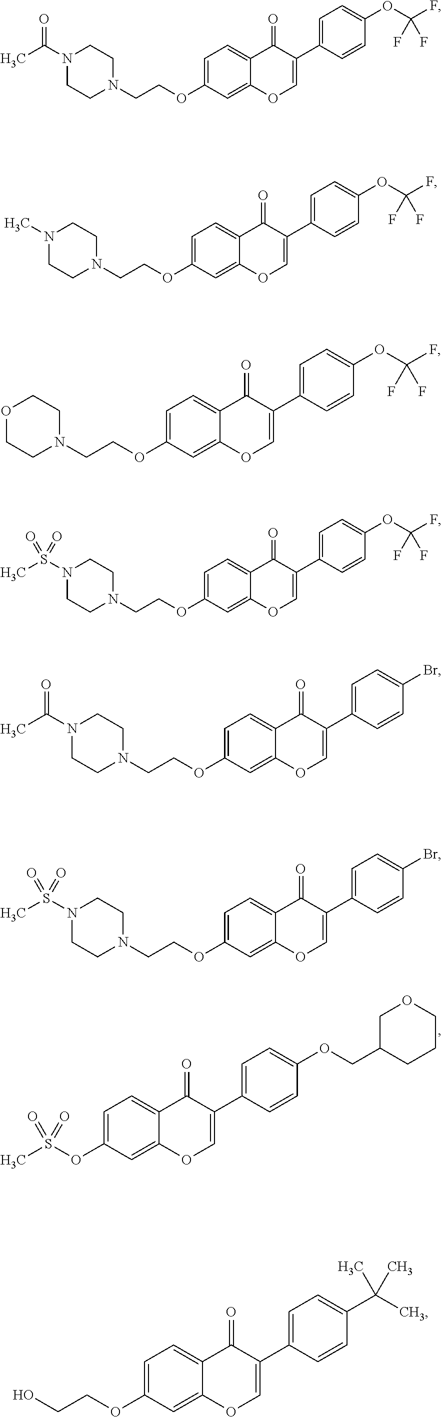

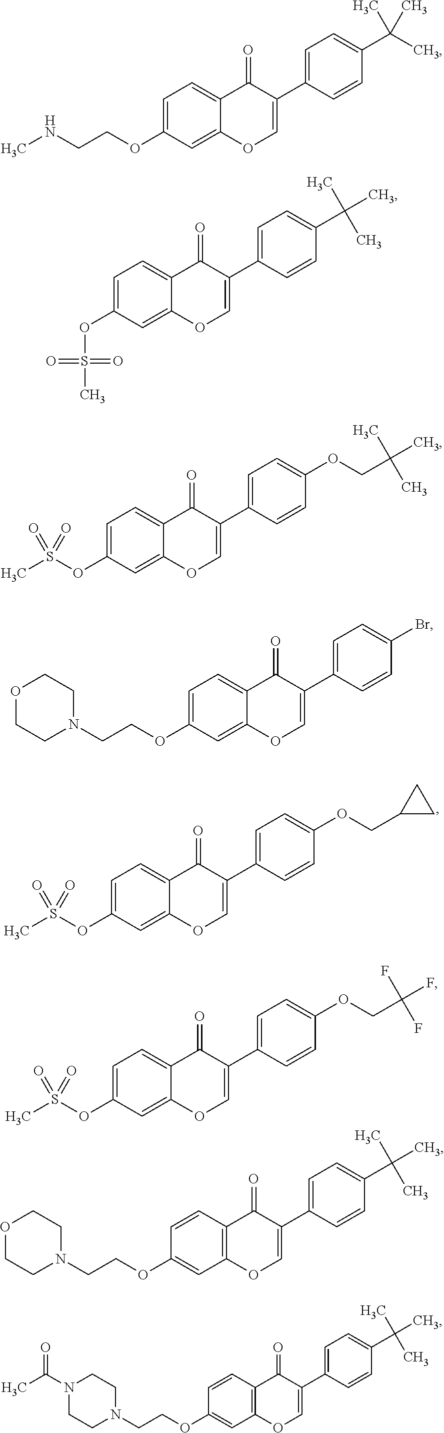

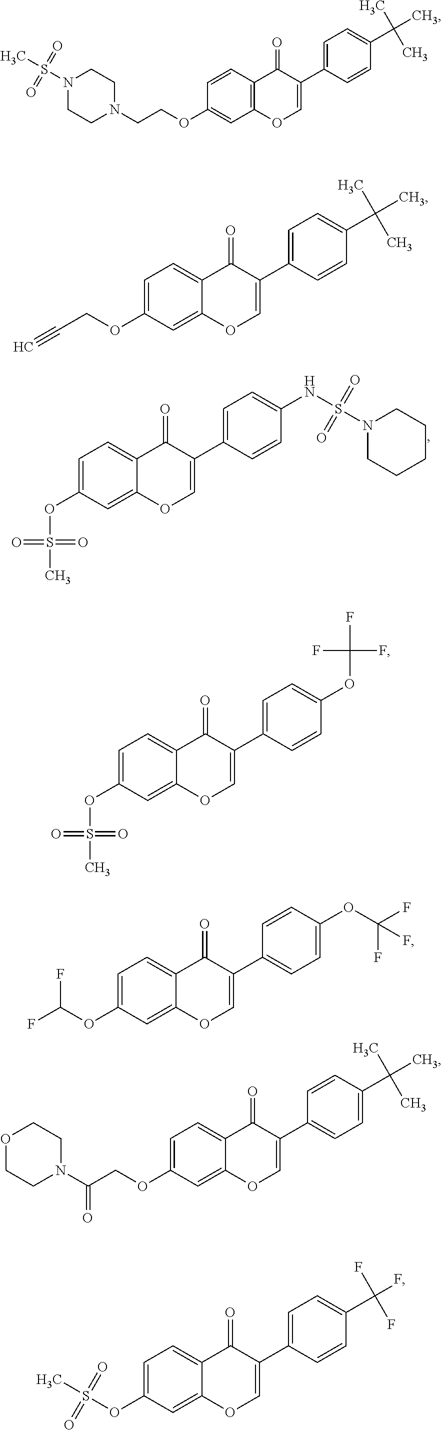

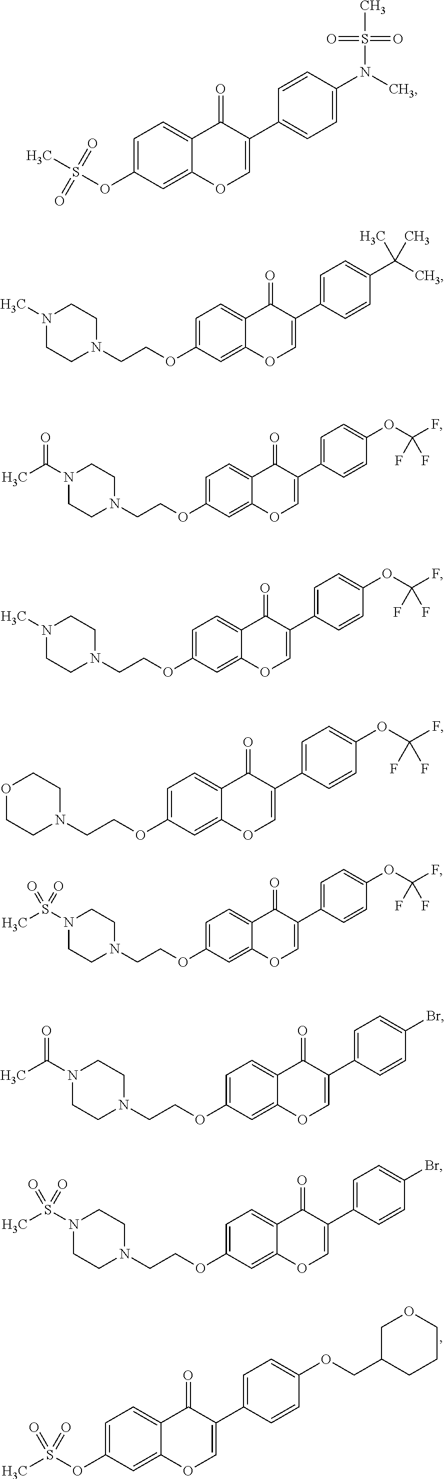

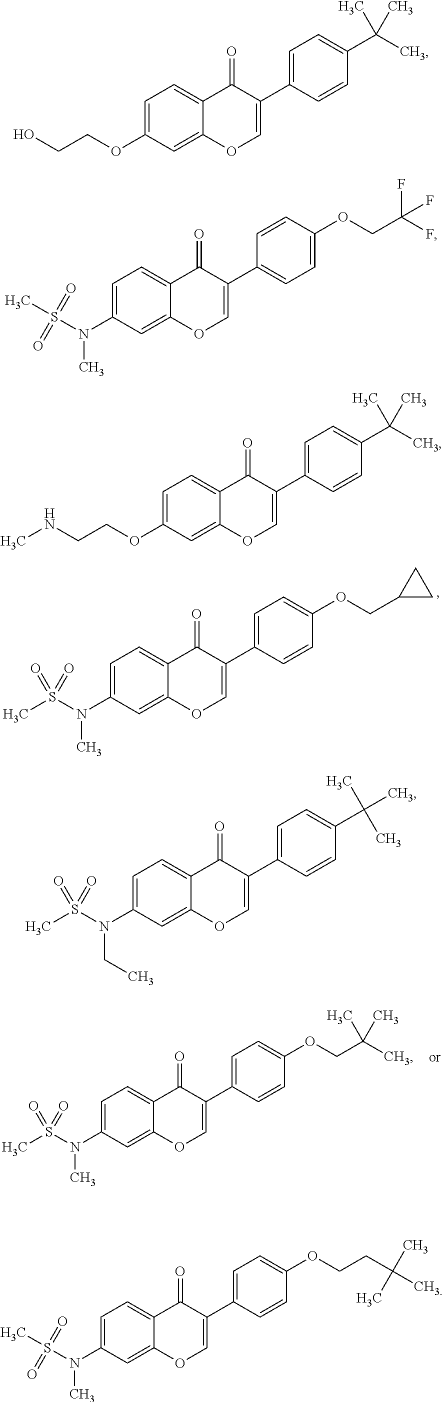

21. A method of treating a viral infection in a subject comprising administering to the subject a therapeutically effective amount of a pharmaceutical composition of claim 14 thereby treating the viral infection in the subject.

22. A method of claim 21, wherein the viral infection is caused by a virus from one or more of the following families: Arenaviridae, Arterivirus, Astroviridae, Birnaviridae, Bromoviridae, Bunyaviridae, Caliciviridae, Closteroviridae, Comoviridae, Coronaviridae, Cystoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Paramyxoviridae, Picobirnaviridae, Picobirnavirus, Picornaviridae, Potyviridae, Reoviridae, Retroviridae, Roniviridae, Sequiviridae, Tenuivirus, Togaviridae, Tombusviridae, Totiviridae, or Tymoviridae.

23. A method of claim 21, wherein the viral infection is caused by one or more of: influenza virus, Alfuy virus, Banzi virus, bovine diarrhea virus, Chikungunya virus, Dengue virus (DNV), Hepatitis B virus (HBV), Hepatitis C virus (HCV), human cytomegalovirus (hCMV), human immunodeficiency virus (HIV), Ilheus virus, influenza virus (including avian and swine isolates), Japanese encephalitis virus, Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), or yellow fever virus.

24. A method of claim 21, wherein the pharmaceutical composition is administered as an adjuvant for a prophylactic or therapeutic vaccine.

25. A method of claim 24, wherein the method comprises vaccinating a subject by additionally administering a vaccine against: Alfuy virus, Banzi virus, bovine diarrhea virus, Chikungunya virus, DNV, HBV, HCV, hCMV, HIV, Ilheus virus, influenza virus (including avian and swine isolates), Japanese encephalitis virus, Kokobera virus, Kunjin virus, Kyasanur forest disease virus, louping-ill virus, measles virus, MERS, metapneumovirus, any of the Mosaic Viruses, Murray Valley virus, parainfluenza virus, poliovirus, Powassan virus, RSV, Rocio virus, SARS, St. Louis encephalitis virus, tick-borne encephalitis virus, WNV, or yellow fever virus.

26. A method of modulating the innate immune response in a eukaryotic cell, comprising administering to the cell a compound of claim 4.

27. A method of claim 26, wherein the cell is in vivo.

28. A method of claim 26, wherein the cell is in vitro.

29. A pharmaceutical composition comprising a compound of claim 4.

30. A pharmaceutical composition of claim 29, for use in therapy.

31. A pharmaceutical composition of claim 29, for use in treating or preventing a viral infection in a subject.

32. A pharmaceutical composition for use according to claim 30, wherein said pharmaceutical composition is administered as an adjuvant for a prophylactic or therapeutic vaccine.

33. A method of treating a viral infection in a subject comprising administering to the subject a therapeutically effective amount of a pharmaceutical composition of claim 29 thereby treating the viral infection in the subject.

34. A method of claim 33, wherein the viral infection is caused by a virus from one or more of the following families: Arenaviridae, Arterivirus, Astroviridae, Birnaviridae, Bromoviridae, Bunyaviridae, Caliciviridae, Closteroviridae, Comoviridae, Coronaviridae, Cystoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Paramyxoviridae, Picobirnaviridae, Picobirnavirus, Picornaviridae, Potyviridae, Reoviridae, Retroviridae, Roniviridae, Sequiviridae, Tenuivirus, Togaviridae, Tombusviridae, Totiviridae, or Tymoviridae.

35. A method of claim 33, wherein the viral infection is caused by one or more of influenza virus, Alfuy virus, Banzi virus, bovine diarrhea virus, Chikungunya virus, Dengue virus (DNV), Hepatitis B virus (HBV), Hepatitis C virus (HCV), human cytomegalovirus (hCMV), human immunodeficiency virus (HIV), Ilheus virus, influenza virus (including avian and swine isolates), Japanese encephalitis virus, Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-borne encephalitis virus, West Nile virus (WNV), or yellow fever virus.

36. A method of claim 33, wherein the pharmaceutical composition is administered as an adjuvant for a prophylactic or therapeutic vaccine.

37. A method of claim 36, wherein the method comprises vaccinating a subject by additionally administering a vaccine against Alfuy virus, Banzi virus, bovine diarrhea virus, Chikungunya virus, DNV, HBV, HCV, hCMV, HIV, Ilheus virus, influenza virus (including avian and swine isolates), Japanese encephalitis virus, Kokobera virus, Kunjin virus, Kyasanur forest disease virus, louping-ill virus, measles virus, MERS, metapneumovirus, any of the Mosaic Viruses, Murray Valley virus, parainfluenza virus, poliovirus, Powassan virus, RSV, Rocio virus, SARS, St. Louis encephalitis virus, tick-borne encephalitis virus, WNV, or yellow fever virus.
